SEQUENCE LISTING (76 sequences)

SEQ ID NO: 1 - DNA, 1494 bp, Artificial Sequence
  Chemically Synthesized, CD19DBiTE, without start or IgE leader
gatattcagctgactcagagccctgcttccctggccgtgtccctgggacagagggcaaccatctcttgcaaggcctcccagtctgtggactacgatggcgactcctatctgaactggtaccagcagatcccaggccagccccctaagctgctgatctatgacgcctctaatctggtgagcggcatcccacccagattcagcggctccggctctggcacagacttcaccctgaacatccacccagtggagaaggtggacgcagcaacctaccactgccagcagtccacagaggatccttggaccttcggcggcggcacaaagctggagatcaagggcggcggcggcagcggcggcggcggcagcggcggcggcggcagccaggtgcagctgcagcagagcggcgccgagctggtgcggcccggcagctccgtgaagatctcctgtaaggcctctggctatgccttttctagctactggatgaattgggtgaagcagcgccccggacagggcctggagtggatcggccagatctggcctggcgatggcgacacaaactacaatggcaagttcaagggcaaggccacactgaccgccgacgagtcctctagcaccgcctacatgcagctgtcctctctggcctccgaggattctgccgtgtatttttgcgccaggagagagacaacaaccgtgggcaggtactattacgccatggactactggggccagggcacaaccgtgacagtgagcagcggcggcggcggctctgatatcaagctgcagcagagcggagcagagctggccagacctggagccagcgtgaagatgtcctgtaagacctctggctatacattcacccggtacacaatgcactgggtgaagcagcgcccaggccagggcctggaatggatcggctacatcaaccccagccggggctataccaactacaatcagaagtttaaggataaggccaccctgacaaccgacaagtctagctccacagcctatatgcagctgtctagcctgaccagcgaggactccgccgtgtattactgcgcccgctattacgacgatcactattgtctggattattggggacagggcacaaccctgacagtgtcctctgtggagggcggctctggcggcagcggcggctccggcggctctggcggcgtggacgatatccagctgacccagtcccctgcaatcatgagcgcctccccaggagagaaggtgacaatgacctgcagggccagctcctctgtgagctatatgaattggtaccagcagaagagcggcacatcccctaagaggtggatctatgacaccagcaaggtggcctccggcgtgccatacagattctctggcagcggctccggcacctcttatagcctgacaatcagctccatggaggccgaggatgccgccacctattactgtcagcagtggtctagcaatcctctgactttcggggctgggacaaaactggaactgaag

SEQ ID NO: 2 - protein, 498 aa, Artificial Sequence
  Chemically Synthesized, CD19DBiTE, without start or IgE leader
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 3 - DNA, 1554 bp, Artificial Sequence
  Chemically Synthesized, CD19DBiTE, operably linked to IgE leader
atggactggacttggatcctgttcctggtcgccgccgccacacgggtgcattccgatattcagctgactcagagccctgcttccctggccgtgtccctgggacagagggcaaccatctcttgcaaggcctcccagtctgtggactacgatggcgactcctatctgaactggtaccagcagatcccaggccagccccctaagctgctgatctatgacgcctctaatctggtgagcggcatcccacccagattcagcggctccggctctggcacagacttcaccctgaacatccacccagtggagaaggtggacgcagcaacctaccactgccagcagtccacagaggatccttggaccttcggcggcggcacaaagctggagatcaagggcggcggcggcagcggcggcggcggcagcggcggcggcggcagccaggtgcagctgcagcagagcggcgccgagctggtgcggcccggcagctccgtgaagatctcctgtaaggcctctggctatgccttttctagctactggatgaattgggtgaagcagcgccccggacagggcctggagtggatcggccagatctggcctggcgatggcgacacaaactacaatggcaagttcaagggcaaggccacactgaccgccgacgagtcctctagcaccgcctacatgcagctgtcctctctggcctccgaggattctgccgtgtatttttgcgccaggagagagacaacaaccgtgggcaggtactattacgccatggactactggggccagggcacaaccgtgacagtgagcagcggcggcggcggctctgatatcaagctgcagcagagcggagcagagctggccagacctggagccagcgtgaagatgtcctgtaagacctctggctatacattcacccggtacacaatgcactgggtgaagcagcgcccaggccagggcctggaatggatcggctacatcaaccccagccggggctataccaactacaatcagaagtttaaggataaggccaccctgacaaccgacaagtctagctccacagcctatatgcagctgtctagcctgaccagcgaggactccgccgtgtattactgcgcccgctattacgacgatcactattgtctggattattggggacagggcacaaccctgacagtgtcctctgtggagggcggctctggcggcagcggcggctccggcggctctggcggcgtggacgatatccagctgacccagtcccctgcaatcatgagcgcctccccaggagagaaggtgacaatgacctgcagggccagctcctctgtgagctatatgaattggtaccagcagaagagcggcacatcccctaagaggtggatctatgacaccagcaaggtggcctccggcgtgccatacagattctctggcagcggctccggcacctcttatagcctgacaatcagctccatggaggccgaggatgccgccacctattactgtcagcagtggtctagcaatcctctgactttcggggctgggacaaaactggaactgaagtgataa

SEQ ID NO: 4 - protein, 516 aa, Artificial Sequence
  Chemically Synthesized, CD19DBiTE, operably linked to IgE leader
MDWTWILFLVAAATRVHSDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 5 - DNA, 1572 bp, Artificial Sequence
  Chemically Synthesized, CD19DBiTE, operably linked to IgE leader and His tag
atggactggacttggatcctgttcctggtcgccgccgccacacgggtgcattccgatattcagctgactcagagccctgcttccctggccgtgtccctgggacagagggcaaccatctcttgcaaggcctcccagtctgtggactacgatggcgactcctatctgaactggtaccagcagatcccaggccagccccctaagctgctgatctatgacgcctctaatctggtgagcggcatcccacccagattcagcggctccggctctggcacagacttcaccctgaacatccacccagtggagaaggtggacgcagcaacctaccactgccagcagtccacagaggatccttggaccttcggcggcggcacaaagctggagatcaagggcggcggcggcagcggcggcggcggcagcggcggcggcggcagccaggtgcagctgcagcagagcggcgccgagctggtgcggcccggcagctccgtgaagatctcctgtaaggcctctggctatgccttttctagctactggatgaattgggtgaagcagcgccccggacagggcctggagtggatcggccagatctggcctggcgatggcgacacaaactacaatggcaagttcaagggcaaggccacactgaccgccgacgagtcctctagcaccgcctacatgcagctgtcctctctggcctccgaggattctgccgtgtatttttgcgccaggagagagacaacaaccgtgggcaggtactattacgccatggactactggggccagggcacaaccgtgacagtgagcagcggcggcggcggctctgatatcaagctgcagcagagcggagcagagctggccagacctggagccagcgtgaagatgtcctgtaagacctctggctatacattcacccggtacacaatgcactgggtgaagcagcgcccaggccagggcctggaatggatcggctacatcaaccccagccggggctataccaactacaatcagaagtttaaggataaggccaccctgacaaccgacaagtctagctccacagcctatatgcagctgtctagcctgaccagcgaggactccgccgtgtattactgcgcccgctattacgacgatcactattgtctggattattggggacagggcacaaccctgacagtgtcctctgtggagggcggctctggcggcagcggcggctccggcggctctggcggcgtggacgatatccagctgacccagtcccctgcaatcatgagcgcctccccaggagagaaggtgacaatgacctgcagggccagctcctctgtgagctatatgaattggtaccagcagaagagcggcacatcccctaagaggtggatctatgacaccagcaaggtggcctccggcgtgccatacagattctctggcagcggctccggcacctcttatagcctgacaatcagctccatggaggccgaggatgccgccacctattactgtcagcagtggtctagcaatcctctgactttcggggctgggacaaaactggaactgaagcaccaccaccaccaccattgataa

SEQ ID NO: 6 - protein, 522 aa, Artificial Sequence
  Chemically Synthesized, CD19DBiTE, operably linked to IgE leader and His tag
MDWTWILFLVAAATRVHSDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 7 - DNA, 1503 bp, Artificial Sequence
  Chemically Synthesized, BCMADBiTE, without start, stop or IgE leader
caggtgcagctggtgcagtcaggagccgaggtcaagaagcccggcgcctccgtgaaggtgtcttgcaaggcctccggctattctttccctgattactatatcaactgggtgcggcaggcaccaggacagggcctggagtggatgggctggatctatttcgccagcggcaactccgagtacaatcagaagtttacaggccgggtgaccatgacacgcgacacaagctcctctaccgcctacatggagctgagctccctgcgctccgaggataccgccgtgtatttctgcgcctctctgtacgactatgattggtactttgacgtgtggggccagggcacaatggtgaccgtgtctagcggcggcggcggcagcggcggcggcggcagcggcggcggcggctccgatatcgtgatgacccagacacctctgtccctgtctgtgacacccggcgagcctgccagcatctcctgtaagtcctctcagagcctggtgcactccaacggcaatacctatctgcactggtacctgcagaagcctggccagtccccacagctgctgatctacaaggtgtctaaccggttcagcggcgtgccagaccgcttttctggaagcggatccggagcagatttcacactgaagatctctagggtggaggccgaggacgtgggcgtgtactattgcgccgagacaagccacgtgccctggacatttggccagggcaccaagctggagatcaagagcggcggcggcggctctgaggtgcagctggtggagagcggcggcggcctggtgcagcccggcggctccctgaagctgtcttgtgccgccagcggcttcaccttcaacaagtatgccatgaattgggtgaggcaggcacctggcaagggcctggagtgggtggccaggatcagaagcaagtacaacaattatgccacctactatgccgactccgtgaaggataggttcacaatctccagagacgattctaagaataccgcctacctgcagatgaacaatctgaagacagaggataccgccgtgtactattgcgtgagacacggcaactttggcaattcttacatcagctattgggcctactggggccagggcacactggtgaccgtgagcagcggcggcggcggcagcggcggcggcggcagcggcggcggcggcagccagacagtggtgacccaggagccaagcctgaccgtgtcccctggcggcaccgtgacactgacctgtggatctagcacaggagcagtgacctccggaaactaccctaattgggtgcagcagaagccaggacaggcccctaggggcctgatcggcggaacaaagttcctggccccaggcacccccgccagattttctggcagcctgctgggcggcaaggccgccctgaccctgtccggagtgcagccagaggacgaggccgagtactattgcgtgctgtggtacagcaatcgctgggtcttcggagggggaaccaaactgactgtcctg

SEQ ID NO: 8 - protein, 501 aa, Artificial Sequence
  Chemically Synthesized, BCMADBiTE, without start or IgE leader
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSSGGGGSGGGGSGGGGSDIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 9 - DNA, 1563 bp, Artificial Sequence
  Chemically Synthesized, BCMADBiTE, operably linked to IgE leader
atggattggacatggattctgtttctggtcgccgccgctacccgcgtgcatagtcaggtgcagctggtgcagtcaggagccgaggtcaagaagcccggcgcctccgtgaaggtgtcttgcaaggcctccggctattctttccctgattactatatcaactgggtgcggcaggcaccaggacagggcctggagtggatgggctggatctatttcgccagcggcaactccgagtacaatcagaagtttacaggccgggtgaccatgacacgcgacacaagctcctctaccgcctacatggagctgagctccctgcgctccgaggataccgccgtgtatttctgcgcctctctgtacgactatgattggtactttgacgtgtggggccagggcacaatggtgaccgtgtctagcggcggcggcggcagcggcggcggcggcagcggcggcggcggctccgatatcgtgatgacccagacacctctgtccctgtctgtgacacccggcgagcctgccagcatctcctgtaagtcctctcagagcctggtgcactccaacggcaatacctatctgcactggtacctgcagaagcctggccagtccccacagctgctgatctacaaggtgtctaaccggttcagcggcgtgccagaccgcttttctggaagcggatccggagcagatttcacactgaagatctctagggtggaggccgaggacgtgggcgtgtactattgcgccgagacaagccacgtgccctggacatttggccagggcaccaagctggagatcaagagcggcggcggcggctctgaggtgcagctggtggagagcggcggcggcctggtgcagcccggcggctccctgaagctgtcttgtgccgccagcggcttcaccttcaacaagtatgccatgaattgggtgaggcaggcacctggcaagggcctggagtgggtggccaggatcagaagcaagtacaacaattatgccacctactatgccgactccgtgaaggataggttcacaatctccagagacgattctaagaataccgcctacctgcagatgaacaatctgaagacagaggataccgccgtgtactattgcgtgagacacggcaactttggcaattcttacatcagctattgggcctactggggccagggcacactggtgaccgtgagcagcggcggcggcggcagcggcggcggcggcagcggcggcggcggcagccagacagtggtgacccaggagccaagcctgaccgtgtcccctggcggcaccgtgacactgacctgtggatctagcacaggagcagtgacctccggaaactaccctaattgggtgcagcagaagccaggacaggcccctaggggcctgatcggcggaacaaagttcctggccccaggcacccccgccagattttctggcagcctgctgggcggcaaggccgccctgaccctgtccggagtgcagccagaggacgaggccgagtactattgcgtgctgtggtacagcaatcgctgggtcttcggagggggaaccaaactgactgtcctgtgataa

SEQ ID NO: 10 - protein, 519 aa, Artificial Sequence
  Chemically Synthesized, BCMADBiTE, operably linked to IgE leader
MDWTWILFLVAAATRVHSQVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSSGGGGSGGGGSGGGGSDIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 11 - DNA, 1587 bp, Artificial Sequence
  Chemically Synthesized, BCMADBiTE, operably linked to IgE leader and His tag
atggattggacatggattctgtttctggtcgccgccgctacccgcgtgcatagtcaggtgcagctggtgcagtcaggagccgaggtcaagaagcccggcgcctccgtgaaggtgtcttgcaaggcctccggctattctttccctgattactatatcaactgggtgcggcaggcaccaggacagggcctggagtggatgggctggatctatttcgccagcggcaactccgagtacaatcagaagtttacaggccgggtgaccatgacacgcgacacaagctcctctaccgcctacatggagctgagctccctgcgctccgaggataccgccgtgtatttctgcgcctctctgtacgactatgattggtactttgacgtgtggggccagggcacaatggtgaccgtgtctagcggcggcggcggcagcggcggcggcggcagcggcggcggcggctccgatatcgtgatgacccagacacctctgtccctgtctgtgacacccggcgagcctgccagcatctcctgtaagtcctctcagagcctggtgcactccaacggcaatacctatctgcactggtacctgcagaagcctggccagtccccacagctgctgatctacaaggtgtctaaccggttcagcggcgtgccagaccgcttttctggaagcggatccggagcagatttcacactgaagatctctagggtggaggccgaggacgtgggcgtgtactattgcgccgagacaagccacgtgccctggacatttggccagggcaccaagctggagatcaagagcggcggcggcggctctgaggtgcagctggtggagagcggcggcggcctggtgcagcccggcggctccctgaagctgtcttgtgccgccagcggcttcaccttcaacaagtatgccatgaattgggtgaggcaggcacctggcaagggcctggagtgggtggccaggatcagaagcaagtacaacaattatgccacctactatgccgactccgtgaaggataggttcacaatctccagagacgattctaagaataccgcctacctgcagatgaacaatctgaagacagaggataccgccgtgtactattgcgtgagacacggcaactttggcaattcttacatcagctattgggcctactggggccagggcacactggtgaccgtgagcagcggcggcggcggcagcggcggcggcggcagcggcggcggcggcagccagacagtggtgacccaggagccaagcctgaccgtgtcccctggcggcaccgtgacactgacctgtggatctagcacaggagcagtgacctccggaaactaccctaattgggtgcagcagaagccaggacaggcccctaggggcctgatcggcggaacaaagttcctggccccaggcacccccgccagattttctggcagcctgctgggcggcaaggccgccctgaccctgtccggagtgcagccagaggacgaggccgagtactattgcgtgctgtggtacagcaatcgctgggtcttcggagggggaaccaaactgactgtcctgtgataacaccaccaccaccaccattgataa

SEQ ID NO: 12 - protein, 525 aa, Artificial Sequence
  Chemically Synthesized, BCMADBiTE, operably linked to IgE leader and His tag
MDWTWILFLVAAATRVHSQVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSSGGGGSGGGGSGGGGSDIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 13 - DNA, 1515 bp, Artificial Sequence
  Chemically Synthesized, CD33BiTE, without stop, start or IgE leader
caggtgcagctggtgcagtcaggggctgaagtcaagaagcccggcgagtccgtgaaggtgtcttgcaaggccagcggctataccttcacaaactacggcatgaattgggtgaagcaggcccctggccagtgtctggagtggatgggctggatcaacacctatacaggcgagccaacctacgccgataagtttcagggcagggtgacaatgaccacagacacctccacatctaccgcctacatggagatccggaatctgggcggcgacgataccgccgtgtactattgcgcccgctggagctggtccgatggctactacgtgtacttcgactattggggccagggcacctccgtgacagtgagcagcggcggcggcggcagcggcggcggcggcagcggcggcggcggctccgatatcgtgatgacccagtcccccgactctctgacagtgagcctgggcgagagaaccacaatcaactgtaagtctagccagagcgtgctggattcctctaccaacaagaacagcctggcatggtaccagcagaagccaggacagccccctaagctgctgctgagctgggcatccaccagggagtccggcatccctgacagattctctggcagcggctccggcacagactttacactgaccatcgattccccacagcccgaggactctgccacctactattgccagcagtccgcccacttccctatcacctttggctgtggcacacggctggagatcaagagcggcggcggcggctctgaggtgcagctggtggagagcggcggcggcctggtgcagccaggcggctccctgaagctgtcttgcgcagcaagcggcttcaccttcaacaagtatgcaatgaattgggtgcgccaggcaccaggcaagggcctggagtgggtggccaggatcagatctaagtacaacaattatgccacctactatgccgacagcgtgaaggatcggttcaccatctcccgcgacgattctaagaatacagcctacctgcagatgaacaatctgaagaccgaggatacagccgtgtactattgcgtgaggcacggcaactttggcaattcttacatcagctattgggcatactggggacagggcaccctggtcacagtgagcagcggcggcggcggcagcggcggcggcggcagcggcggcggcggcagccagaccgtggtgacacaggagccaagcctgaccgtgtcccctggcggcacagtgaccctgacatgtggatctagcaccggagcagtgacaagcggaaactatcctaattgggtgcagcagaaacctggacaggcccctaggggcctgatcggcggaaccaagttcctggcccctggcacaccagccagattttctggcagcctgctgggcggcaaggccgccctgaccctgtccggagtgcagccagaggacgaggccgagtactattgcgtgctgtggtactctaatcgctgggtcttcgggggagggacaaaactgactgtgctg

SEQ ID NO: 14 - protein, 505 aa, Artificial Sequence
  Chemically Synthesized, CD33BiTE, without start or IgE leader
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 15 - DNA, 1575 bp, Artificial Sequence
  Chemically Synthesized, CD33DBiTE, operably linked to IgE leader
atggattggacatggattctgtttctggtcgccgccgcaactcgcgtccactcacaggtgcagctggtgcagtcaggggctgaagtcaagaagcccggcgagtccgtgaaggtgtcttgcaaggccagcggctataccttcacaaactacggcatgaattgggtgaagcaggcccctggccagtgtctggagtggatgggctggatcaacacctatacaggcgagccaacctacgccgataagtttcagggcagggtgacaatgaccacagacacctccacatctaccgcctacatggagatccggaatctgggcggcgacgataccgccgtgtactattgcgcccgctggagctggtccgatggctactacgtgtacttcgactattggggccagggcacctccgtgacagtgagcagcggcggcggcggcagcggcggcggcggcagcggcggcggcggctccgatatcgtgatgacccagtcccccgactctctgacagtgagcctgggcgagagaaccacaatcaactgtaagtctagccagagcgtgctggattcctctaccaacaagaacagcctggcatggtaccagcagaagccaggacagccccctaagctgctgctgagctgggcatccaccagggagtccggcatccctgacagattctctggcagcggctccggcacagactttacactgaccatcgattccccacagcccgaggactctgccacctactattgccagcagtccgcccacttccctatcacctttggctgtggcacacggctggagatcaagagcggcggcggcggctctgaggtgcagctggtggagagcggcggcggcctggtgcagccaggcggctccctgaagctgtcttgcgcagcaagcggcttcaccttcaacaagtatgcaatgaattgggtgcgccaggcaccaggcaagggcctggagtgggtggccaggatcagatctaagtacaacaattatgccacctactatgccgacagcgtgaaggatcggttcaccatctcccgcgacgattctaagaatacagcctacctgcagatgaacaatctgaagaccgaggatacagccgtgtactattgcgtgaggcacggcaactttggcaattcttacatcagctattgggcatactggggacagggcaccctggtcacagtgagcagcggcggcggcggcagcggcggcggcggcagcggcggcggcggcagccagaccgtggtgacacaggagccaagcctgaccgtgtcccctggcggcacagtgaccctgacatgtggatctagcaccggagcagtgacaagcggaaactatcctaattgggtgcagcagaaacctggacaggcccctaggggcctgatcggcggaaccaagttcctggcccctggcacaccagccagattttctggcagcctgctgggcggcaaggccgccctgaccctgtccggagtgcagccagaggacgaggccgagtactattgcgtgctgtggtactctaatcgctgggtcttcgggggagggacaaaactgactgtgctgtgataa

SEQ ID NO: 16 - protein, 523 aa, Artificial Sequence
  Chemically Synthesized, CD33DBiTE, operably linked to IgE leader
MDWTWILFLVAAATRVHSQVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 17 - DNA, 1593 bp, Artificial Sequence
  Chemically Synthesized, CD33DBiTE, operably linked to IgE leader and His tag
atggattggacatggattctgtttctggtcgccgccgcaactcgcgtccactcacaggtgcagctggtgcagtcaggggctgaagtcaagaagcccggcgagtccgtgaaggtgtcttgcaaggccagcggctataccttcacaaactacggcatgaattgggtgaagcaggcccctggccagtgtctggagtggatgggctggatcaacacctatacaggcgagccaacctacgccgataagtttcagggcagggtgacaatgaccacagacacctccacatctaccgcctacatggagatccggaatctgggcggcgacgataccgccgtgtactattgcgcccgctggagctggtccgatggctactacgtgtacttcgactattggggccagggcacctccgtgacagtgagcagcggcggcggcggcagcggcggcggcggcagcggcggcggcggctccgatatcgtgatgacccagtcccccgactctctgacagtgagcctgggcgagagaaccacaatcaactgtaagtctagccagagcgtgctggattcctctaccaacaagaacagcctggcatggtaccagcagaagccaggacagccccctaagctgctgctgagctgggcatccaccagggagtccggcatccctgacagattctctggcagcggctccggcacagactttacactgaccatcgattccccacagcccgaggactctgccacctactattgccagcagtccgcccacttccctatcacctttggctgtggcacacggctggagatcaagagcggcggcggcggctctgaggtgcagctggtggagagcggcggcggcctggtgcagccaggcggctccctgaagctgtcttgcgcagcaagcggcttcaccttcaacaagtatgcaatgaattgggtgcgccaggcaccaggcaagggcctggagtgggtggccaggatcagatctaagtacaacaattatgccacctactatgccgacagcgtgaaggatcggttcaccatctcccgcgacgattctaagaatacagcctacctgcagatgaacaatctgaagaccgaggatacagccgtgtactattgcgtgaggcacggcaactttggcaattcttacatcagctattgggcatactggggacagggcaccctggtcacagtgagcagcggcggcggcggcagcggcggcggcggcagcggcggcggcggcagccagaccgtggtgacacaggagccaagcctgaccgtgtcccctggcggcacagtgaccctgacatgtggatctagcaccggagcagtgacaagcggaaactatcctaattgggtgcagcagaaacctggacaggcccctaggggcctgatcggcggaaccaagttcctggcccctggcacaccagccagattttctggcagcctgctgggcggcaaggccgccctgaccctgtccggagtgcagccagaggacgaggccgagtactattgcgtgctgtggtactctaatcgctgggtcttcgggggagggacaaaactgactgtgctgcatcaccaccaccaccattgataa

SEQ ID NO: 18 - protein, 529 aa, Artificial Sequence
  Chemically Synthesized, CD33DBiTE, operably linked to IgE leader and His tag
MDWTWILFLVAAATRVHSQVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 19 - DNA, 1482 bp, Artificial Sequence
  Chemically Synthesized, FAPBiTE, without start, stop or IgE leader
gaagtgcagctgctggaaagtggcgggggcctggtccagcctggcgggtccctgagactgagttgtgctgcctccgggtttacatttagctcctacgcaatgtcctgggtgaggcaggcaccaggcaagggcctggagtgggtgtctgccatctccggctctggcggctctacctactatgccgacagcgtgaagggcaggttcaccatcagcagagataactccaagaatacactgtacctgcagatgaacagcctgagggccgaggacacagccgtgtactattgcgccaagggctggctgggcaattttgattattggggccagggcaccctggtgacagtgtctagcggcggcggcggcagcggcggcggcggcagcggcggcggcggctccgagatcgtgctgacccagagcccaggcacactgagcctgtccccaggagagagggccaccctgtcctgtcgcgcctctcagagcgtgtcccggtcttacctggcctggtatcagcagaagccaggacaggcccctcgcctgctgatcatcggagcatctaccagggcaacaggcatccctgacagattcagcggctccggctctggcacagacttcaccctgacaatctccagactggagccagaggacttcgccgtgtactattgtcagcaggggcaggtcattccacccacattcgggcagggcactaaagtcgagatcaaaggcggcggcggctctgaggtgcagctggtggagagcggcggcggcctggtgcagcccggcggctccctgaagctgtcttgtgccgccagcggcttcaccttcaacaagtatgccatgaattgggtgaggcaggcacctggcaagggcctggagtgggtggccaggatcagaagcaagtacaacaattatgccacctactatgccgactccgtgaaggataggttcacaatctccagagacgattctaagaataccgcctacctgcagatgaacaatctgaagacagaggataccgccgtgtactattgcgtgagacacggcaactttggcaattcttacatcagctattgggcctactggggccagggcacactggtgaccgtgagcagcggcggcggcggcagcggcggcggcggcagcggcggcggcggcagccagacagtggtgacccaggagccaagcctgaccgtgtcccctggcggcaccgtgacactgacctgtggatctagcacaggagcagtgacctccggaaactaccctaattgggtgcagcagaagccaggacaggcccctaggggcctgatcggcggaacaaagttcctggccccaggcacccccgccagattttctggcagcctgctgggcggcaaggccgccctgaccctgtccggagtgcagccagaggacgaggccgagtactattgcgtgctgtggtacagcaatcgctgggtcttcggagggggaaccaaactgactgtcctg

SEQ ID NO: 20 - protein, 493 aa, Artificial Sequence
  Chemically Synthesized, FAPBiTE, without start or IgE leader
VQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGWLGNFDYWGQGTLVTVSSGGGGSGGGGSGGGGSEIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIKGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 21 - DNA, 1542 bp, Artificial Sequence
  Chemically Synthesized, FAPDBiTE, operably linked to IgE leader
atggattggacatggattctgtttctggtcgccgccgctacccgcgtgcatagtgaagtgcagctgctggaaagtggcgggggcctggtccagcctggcgggtccctgagactgagttgtgctgcctccgggtttacatttagctcctacgcaatgtcctgggtgaggcaggcaccaggcaagggcctggagtgggtgtctgccatctccggctctggcggctctacctactatgccgacagcgtgaagggcaggttcaccatcagcagagataactccaagaatacactgtacctgcagatgaacagcctgagggccgaggacacagccgtgtactattgcgccaagggctggctgggcaattttgattattggggccagggcaccctggtgacagtgtctagcggcggcggcggcagcggcggcggcggcagcggcggcggcggctccgagatcgtgctgacccagagcccaggcacactgagcctgtccccaggagagagggccaccctgtcctgtcgcgcctctcagagcgtgtcccggtcttacctggcctggtatcagcagaagccaggacaggcccctcgcctgctgatcatcggagcatctaccagggcaacaggcatccctgacagattcagcggctccggctctggcacagacttcaccctgacaatctccagactggagccagaggacttcgccgtgtactattgtcagcaggggcaggtcattccacccacattcgggcagggcactaaagtcgagatcaaaggcggcggcggctctgaggtgcagctggtggagagcggcggcggcctggtgcagcccggcggctccctgaagctgtcttgtgccgccagcggcttcaccttcaacaagtatgccatgaattgggtgaggcaggcacctggcaagggcctggagtgggtggccaggatcagaagcaagtacaacaattatgccacctactatgccgactccgtgaaggataggttcacaatctccagagacgattctaagaataccgcctacctgcagatgaacaatctgaagacagaggataccgccgtgtactattgcgtgagacacggcaactttggcaattcttacatcagctattgggcctactggggccagggcacactggtgaccgtgagcagcggcggcggcggcagcggcggcggcggcagcggcggcggcggcagccagacagtggtgacccaggagccaagcctgaccgtgtcccctggcggcaccgtgacactgacctgtggatctagcacaggagcagtgacctccggaaactaccctaattgggtgcagcagaagccaggacaggcccctaggggcctgatcggcggaacaaagttcctggccccaggcacccccgccagattttctggcagcctgctgggcggcaaggccgccctgaccctgtccggagtgcagccagaggacgaggccgagtactattgcgtgctgtggtacagcaatcgctgggtcttcggagggggaaccaaactgactgtcctgtgataa

SEQ ID NO: 22 - protein, 512 aa, Artificial Sequence
  Chemically Synthesized, FAPDBiTE, operably linked to IgE leader
MDWTWILFLVAAATRVHSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGWLGNFDYWGQGTLVTVSSGGGGSGGGGSGGGGSEIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIKGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 23 - DNA, 1560 bp, Artificial Sequence
  Chemically Synthesized, FAPDBiTE, operably linked to IgE leader and His tag
atggattggacatggattctgtttctggtcgccgccgctacccgcgtgcatagtgaagtgcagctgctggaaagtggcgggggcctggtccagcctggcgggtccctgagactgagttgtgctgcctccgggtttacatttagctcctacgcaatgtcctgggtgaggcaggcaccaggcaagggcctggagtgggtgtctgccatctccggctctggcggctctacctactatgccgacagcgtgaagggcaggttcaccatcagcagagataactccaagaatacactgtacctgcagatgaacagcctgagggccgaggacacagccgtgtactattgcgccaagggctggctgggcaattttgattattggggccagggcaccctggtgacagtgtctagcggcggcggcggcagcggcggcggcggcagcggcggcggcggctccgagatcgtgctgacccagagcccaggcacactgagcctgtccccaggagagagggccaccctgtcctgtcgcgcctctcagagcgtgtcccggtcttacctggcctggtatcagcagaagccaggacaggcccctcgcctgctgatcatcggagcatctaccagggcaacaggcatccctgacagattcagcggctccggctctggcacagacttcaccctgacaatctccagactggagccagaggacttcgccgtgtactattgtcagcaggggcaggtcattccacccacattcgggcagggcactaaagtcgagatcaaaggcggcggcggctctgaggtgcagctggtggagagcggcggcggcctggtgcagcccggcggctccctgaagctgtcttgtgccgccagcggcttcaccttcaacaagtatgccatgaattgggtgaggcaggcacctggcaagggcctggagtgggtggccaggatcagaagcaagtacaacaattatgccacctactatgccgactccgtgaaggataggttcacaatctccagagacgattctaagaataccgcctacctgcagatgaacaatctgaagacagaggataccgccgtgtactattgcgtgagacacggcaactttggcaattcttacatcagctattgggcctactggggccagggcacactggtgaccgtgagcagcggcggcggcggcagcggcggcggcggcagcggcggcggcggcagccagacagtggtgacccaggagccaagcctgaccgtgtcccctggcggcaccgtgacactgacctgtggatctagcacaggagcagtgacctccggaaactaccctaattgggtgcagcagaagccaggacaggcccctaggggcctgatcggcggaacaaagttcctggccccaggcacccccgccagattttctggcagcctgctgggcggcaaggccgccctgaccctgtccggagtgcagccagaggacgaggccgagtactattgcgtgctgtggtacagcaatcgctgggtcttcggagggggaaccaaactgactgtcctgcaccaccaccaccaccattgataa

SEQ ID NO: 24 - protein, 518 aa, Artificial Sequence
  Chemically Synthesized, FAPDBiTE, operably linked to IgE leader and His tag
MDWTWILFLVAAATRVHSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGWLGNFDYWGQGTLVTVSSGGGGSGGGGSGGGGSEIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIKGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 25 - DNA, 825 bp, Artificial Sequence
  Chemically Synthesized, FSHRDBiTE, without start or stop codons or IgE leader
tacaccagggacctggtgtataaggatcccgccagacctaagatccagaagacctgcacattcggcggcggcggctctgaggtgcagctggtggagagcggcggcggcctggtgcagcccggcggctccctgaagctgtcttgtgccgccagcggcttcaccttcaacaagtatgccatgaattgggtgaggcaggcacctggcaagggcctggagtgggtggccaggatcagaagcaagtacaacaattatgccacctactatgccgactccgtgaaggataggttcacaatctccagagacgattctaagaataccgcctacctgcagatgaacaatctgaagacagaggataccgccgtgtactattgcgtgagacacggcaactttggcaattcttacatcagctattgggcctactggggccagggcacactggtgaccgtgagcagcggcggcggcggcagcggcggcggcggcagcggcggcggcggcagccagacagtggtgacccaggagccaagcctgaccgtgtcccctggcggcaccgtgacactgacctgtggatctagcacaggagcagtgacctccggaaactaccctaattgggtgcagcagaagccaggacaggcccctaggggcctgatcggcggaacaaagttcctggccccaggcacccccgccagattttctggcagcctgctgggcggcaaggccgccctgaccctgtccggagtgcagccagaggacgaggccgagtactattgcgtgctgtggtacagcaatcgctgggtcttcggagggggaaccaaactgactgtcctg

SEQ ID NO: 26 - protein, 275 aa, Artificial Sequence
  Chemically Synthesized, FSHRDBiTE, without start or IgE leader sequence
YTRDLVYKDPARPKIQKTCTFGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 27 - DNA, 885 bp, Artificial Sequence
  Chemically Synthesized, FSHRDBiTE, operably linked to IgE leader
atggattggacatggattctgtttctggtcgccgccgctacccgcgtgcatagttacaccagggacctggtgtataaggatcccgccagacctaagatccagaagacctgcacattcggcggcggcggctctgaggtgcagctggtggagagcggcggcggcctggtgcagcccggcggctccctgaagctgtcttgtgccgccagcggcttcaccttcaacaagtatgccatgaattgggtgaggcaggcacctggcaagggcctggagtgggtggccaggatcagaagcaagtacaacaattatgccacctactatgccgactccgtgaaggataggttcacaatctccagagacgattctaagaataccgcctacctgcagatgaacaatctgaagacagaggataccgccgtgtactattgcgtgagacacggcaactttggcaattcttacatcagctattgggcctactggggccagggcacactggtgaccgtgagcagcggcggcggcggcagcggcggcggcggcagcggcggcggcggcagccagacagtggtgacccaggagccaagcctgaccgtgtcccctggcggcaccgtgacactgacctgtggatctagcacaggagcagtgacctccggaaactaccctaattgggtgcagcagaagccaggacaggcccctaggggcctgatcggcggaacaaagttcctggccccaggcacccccgccagattttctggcagcctgctgggcggcaaggccgccctgaccctgtccggagtgcagccagaggacgaggccgagtactattgcgtgctgtggtacagcaatcgctgggtcttcggagggggaaccaaactgactgtcctgtgataa

SEQ ID NO: 28 - protein, 293 aa, Artificial Sequence
  Chemically Synthesized, FSHRDBiTE, operably linked to IgE leader
MDWTWILFLVAAATRVHSYTRDLVYKDPARPKIQKTCTFGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 29 - DNA, 903 bp, Artificial Sequence
  Chemically Synthesized, FSHRDBiTE, operably linked to IgE leader and His tag
atggattggacatggattctgtttctggtcgccgccgctacccgcgtgcatagttacaccagggacctggtgtataaggatcccgccagacctaagatccagaagacctgcacattcggcggcggcggctctgaggtgcagctggtggagagcggcggcggcctggtgcagcccggcggctccctgaagctgtcttgtgccgccagcggcttcaccttcaacaagtatgccatgaattgggtgaggcaggcacctggcaagggcctggagtgggtggccaggatcagaagcaagtacaacaattatgccacctactatgccgactccgtgaaggataggttcacaatctccagagacgattctaagaataccgcctacctgcagatgaacaatctgaagacagaggataccgccgtgtactattgcgtgagacacggcaactttggcaattcttacatcagctattgggcctactggggccagggcacactggtgaccgtgagcagcggcggcggcggcagcggcggcggcggcagcggcggcggcggcagccagacagtggtgacccaggagccaagcctgaccgtgtcccctggcggcaccgtgacactgacctgtggatctagcacaggagcagtgacctccggaaactaccctaattgggtgcagcagaagccaggacaggcccctaggggcctgatcggcggaacaaagttcctggccccaggcacccccgccagattttctggcagcctgctgggcggcaaggccgccctgaccctgtccggagtgcagccagaggacgaggccgagtactattgcgtgctgtggtacagcaatcgctgggtcttcggagggggaaccaaactgactgtcctgcaccaccaccaccaccattgataa

SEQ ID NO: 30 - protein, 299 aa, Artificial Sequence
  Chemically Synthesized, FSHRDBiTE, operably linked to IgE leader and His tag
MDWTWILFLVAAATRVHSYTRDLVYKDPARPKIQKTCTFGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 31 - DNA, 1500 bp, Artificial Sequence
  Chemically Synthesized, EGFRBiTE, without start, stop or IgE leader
gagatccagctggtgcagagcggagccgaggtcaagaagcctggggagagcctgagaatttcctgtaaggggagcgggttcaacattgaggactactatatccactgggtgaggcagatgccaggcaagggcctggagtggatgggaagaatcgacccagagaacgatgagacaaagtacggccccatcttccagggccacgtgacaatcagcgccgacacctccatcaatacagtgtatctgcagtggagctccctgaaggcctccgataccgccatgtactattgcgcctttaggggcggcgtgtactggggacagggaaccacagtgacagtgtctagcggcggcggcggcagcggcggcggcggcagcggcggcggcggcagcggcggcggcggctccgacgtggtcatgacccagtctcctgatagcctggccgtgtctctgggagagagggcaacaatcaactgtaagtcctctcagtctctgctggacagcgatggcaagacctatctgaattggctgcagcagaagcctggccagccccctaagcggctgatctccctggtgtctaagctggacagcggcgtgccagatcgcttctctggcagcggctccggcacagactttaccctgacaatcagctccctgcaggccgaggatgtggccgtgtactattgttggcaggggactcattttccagggacattcggaggagggacaaaggtcgagatcaagggcggcggcggctctgaggtgcagctggtggagagcggcggcggcctggtgcagcccggcggctccctgaagctgtcttgtgccgccagcggcttcaccttcaacaagtatgccatgaattgggtgaggcaggcacctggcaagggcctggagtgggtggccaggatcagaagcaagtacaacaattatgccacctactatgccgactccgtgaaggataggttcacaatctccagagacgattctaagaataccgcctacctgcagatgaacaatctgaagacagaggataccgccgtgtactattgcgtgagacacggcaactttggcaattcttacatcagctattgggcctactggggccagggcacactggtgaccgtgagcagcggcggcggcggcagcggcggcggcggcagcggcggcggcggcagccagacagtggtgacccaggagccaagcctgaccgtgtcccctggcggcaccgtgacactgacctgtggatctagcacaggagcagtgacctccggaaactaccctaattgggtgcagcagaagccaggacaggcccctaggggcctgatcggcggaacaaagttcctggccccaggcacccccgccagattttctggcagcctgctgggcggcaaggccgccctgaccctgtccggagtgcagccagaggacgaggccgagtactattgcgtgctgtggtacagcaatcgctgggtcttcggagggggaaccaaactgactgtcctg

SEQ ID NO: 32 - protein, 500 aa, Artificial Sequence
  Chemically Synthesized, EGFRBiTE, without start or IgE leader
EIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSGGGGSGGGGSGGGGSGGGGSDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIKGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 33 - DNA, 1560 bp, Artificial Sequence
  Chemically Synthesized, EGFRDBiTE, operably linked to IgE leader
atggattggacatggattctgtttctggtcgccgccgctacccgcgtgcatagtgagatccagctggtgcagagcggagccgaggtcaagaagcctggggagagcctgagaatttcctgtaaggggagcgggttcaacattgaggactactatatccactgggtgaggcagatgccaggcaagggcctggagtggatgggaagaatcgacccagagaacgatgagacaaagtacggccccatcttccagggccacgtgacaatcagcgccgacacctccatcaatacagtgtatctgcagtggagctccctgaaggcctccgataccgccatgtactattgcgcctttaggggcggcgtgtactggggacagggaaccacagtgacagtgtctagcggcggcggcggcagcggcggcggcggcagcggcggcggcggcagcggcggcggcggctccgacgtggtcatgacccagtctcctgatagcctggccgtgtctctgggagagagggcaacaatcaactgtaagtcctctcagtctctgctggacagcgatggcaagacctatctgaattggctgcagcagaagcctggccagccccctaagcggctgatctccctggtgtctaagctggacagcggcgtgccagatcgcttctctggcagcggctccggcacagactttaccctgacaatcagctccctgcaggccgaggatgtggccgtgtactattgttggcaggggactcattttccagggacattcggaggagggacaaaggtcgagatcaagggcggcggcggctctgaggtgcagctggtggagagcggcggcggcctggtgcagcccggcggctccctgaagctgtcttgtgccgccagcggcttcaccttcaacaagtatgccatgaattgggtgaggcaggcacctggcaagggcctggagtgggtggccaggatcagaagcaagtacaacaattatgccacctactatgccgactccgtgaaggataggttcacaatctccagagacgattctaagaataccgcctacctgcagatgaacaatctgaagacagaggataccgccgtgtactattgcgtgagacacggcaactttggcaattcttacatcagctattgggcctactggggccagggcacactggtgaccgtgagcagcggcggcggcggcagcggcggcggcggcagcggcggcggcggcagccagacagtggtgacccaggagccaagcctgaccgtgtcccctggcggcaccgtgacactgacctgtggatctagcacaggagcagtgacctccggaaactaccctaattgggtgcagcagaagccaggacaggcccctaggggcctgatcggcggaacaaagttcctggccccaggcacccccgccagattttctggcagcctgctgggcggcaaggccgccctgaccctgtccggagtgcagccagaggacgaggccgagtactattgcgtgctgtggtacagcaatcgctgggtcttcggagggggaaccaaactgactgtcctgtgataa

SEQ ID NO: 34 - protein, 518 aa, Artificial Sequence
  Chemically Synthesized, EGFRDBiTE, operably linked to IgE leader
MDWTWILFLVAAATRVHSEIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSGGGGSGGGGSGGGGSGGGGSDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIKGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 35 - DNA, 1578 bp, Artificial Sequence
  Chemically Synthesized, EGFRDBiTE, operably linked to IgE leader and His tag
atggattggacatggattctgtttctggtcgccgccgctacccgcgtgcatagtgagatccagctggtgcagagcggagccgaggtcaagaagcctggggagagcctgagaatttcctgtaaggggagcgggttcaacattgaggactactatatccactgggtgaggcagatgccaggcaagggcctggagtggatgggaagaatcgacccagagaacgatgagacaaagtacggccccatcttccagggccacgtgacaatcagcgccgacacctccatcaatacagtgtatctgcagtggagctccctgaaggcctccgataccgccatgtactattgcgcctttaggggcggcgtgtactggggacagggaaccacagtgacagtgtctagcggcggcggcggcagcggcggcggcggcagcggcggcggcggcagcggcggcggcggctccgacgtggtcatgacccagtctcctgatagcctggccgtgtctctgggagagagggcaacaatcaactgtaagtcctctcagtctctgctggacagcgatggcaagacctatctgaattggctgcagcagaagcctggccagccccctaagcggctgatctccctggtgtctaagctggacagcggcgtgccagatcgcttctctggcagcggctccggcacagactttaccctgacaatcagctccctgcaggccgaggatgtggccgtgtactattgttggcaggggactcattttccagggacattcggaggagggacaaaggtcgagatcaagggcggcggcggctctgaggtgcagctggtggagagcggcggcggcctggtgcagcccggcggctccctgaagctgtcttgtgccgccagcggcttcaccttcaacaagtatgccatgaattgggtgaggcaggcacctggcaagggcctggagtgggtggccaggatcagaagcaagtacaacaattatgccacctactatgccgactccgtgaaggataggttcacaatctccagagacgattctaagaataccgcctacctgcagatgaacaatctgaagacagaggataccgccgtgtactattgcgtgagacacggcaactttggcaattcttacatcagctattgggcctactggggccagggcacactggtgaccgtgagcagcggcggcggcggcagcggcggcggcggcagcggcggcggcggcagccagacagtggtgacccaggagccaagcctgaccgtgtcccctggcggcaccgtgacactgacctgtggatctagcacaggagcagtgacctccggaaactaccctaattgggtgcagcagaagccaggacaggcccctaggggcctgatcggcggaacaaagttcctggccccaggcacccccgccagattttctggcagcctgctgggcggcaaggccgccctgaccctgtccggagtgcagccagaggacgaggccgagtactattgcgtgctgtggtacagcaatcgctgggtcttcggagggggaaccaaactgactgtcctgcaccaccaccaccaccattgataa

SEQ ID NO: 36 - protein, 524 aa, Artificial Sequence
  Chemically Synthesized, EGFRDBiTE, operably linked to IgE leader and His tag
MDWTWILFLVAAATRVHSEIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSGGGGSGGGGSGGGGSGGGGSDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIKGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 37 - DNA, 1494 bp, Artificial Sequence
  Chemically Synthesized, PSMADBiTE without start or stop codons or IgE leader
caggtgcagctggtcgaatctggcggcggcctggtcaagcccggagagagcctgagactgagttgtgcagcaagcgggtttactttctccgactactatatgtactgggtgaggcaggcacctggcaagggcctggagtgggtggcaagcatctccgacggcggatctaacacctactatagcgatatcatcaagggcaggttcacaatctccagagacaacgccaagaacagcctgtacctgcagatgaatagcctgaaggccgaggataccgccgtgtactattgcgcaaggggattcccactgctgaggcacggcgcctttgactattggggccagggcaccctggtgacagtgagcagcggcggcggcggcagcggcggcggcggcagcggcggcggcggctccgatatccagatgacccagtccccatctagcctgtctgccagcgtgggcgacagggtgaccatcacatgtagagcctctcagaacgtggatacaaatgtggcatggtaccagcagaagccaggacaggcccctaagtccctgatctattccgcctcttaccggtatagcgatgtgccatcccgcttcagcggatccgcctctggcaccgactttaccctgacaatctcctctgtgcagagcgaggattttgccacatactattgccagcagtacaactcatacccttacaccttcggaggaggcacaaaactggaaatcaagtcaggcggcggcggctctgaggtgcagctggtggagagcggcggcggcctggtgcagcccggcggctccctgaagctgtcttgtgccgccagcggcttcaccttcaacaagtatgccatgaattgggtgaggcaggcacctggcaagggcctggagtgggtggccaggatcagaagcaagtacaacaattatgccacctactatgccgactccgtgaaggataggttcacaatctccagagacgattctaagaataccgcctacctgcagatgaacaatctgaagacagaggataccgccgtgtactattgcgtgagacacggcaactttggcaattcttacatcagctattgggcctactggggccagggcacactggtgaccgtgagcagcggcggcggcggcagcggcggcggcggcagcggcggcggcggcagccagacagtggtgacccaggagccaagcctgaccgtgtcccctggcggcaccgtgacactgacctgtggatctagcacaggagcagtgacctccggaaactaccctaattgggtgcagcagaagccaggacaggcccctaggggcctgatcggcggaacaaagttcctggccccaggcacccccgccagattttctggcagcctgctgggcggcaaggccgccctgaccctgtccggagtgcagccagaggacgaggccgagtactattgcgtgctgtggtacagcaatcgctgggtcttcggagggggaaccaaactgactgtcctg

SEQ ID NO: 38 - protein, 498 aa, Artificial Sequence
  Chemically Synthesized, PSMADBiTE without start or IgE leader
QVQLVESGGGLVKPGESLRLSCAASGFTFSDYYMYWVRQAPGKGLEWVASISDGGSNTYYSDIIKGRFTISRDNAKNSLYLQMNSLKAEDTAVYYCARGFPLLRHGAFDYWGQGTLVTVSSGGGGSGGGGSGGGGSDIQMTQSPSSLSASVGDRVTITCRASQNVDTNVAWYQQKPGQAPKSLIYSASYRYSDVPSRFSGSASGTDFTLTISSVQSEDFATYYCQQYNSYPYTFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 39 - DNA, 1554 bp, Artificial Sequence
  Chemically Synthesized, PSMADBiTE, operably linked to IgE leader
atggattggacatggattctgtttctggtcgccgccgctacccgcgtgcatagtcaggtgcagctggtcgaatctggcggcggcctggtcaagcccggagagagcctgagactgagttgtgcagcaagcgggtttactttctccgactactatatgtactgggtgaggcaggcacctggcaagggcctggagtgggtggcaagcatctccgacggcggatctaacacctactatagcgatatcatcaagggcaggttcacaatctccagagacaacgccaagaacagcctgtacctgcagatgaatagcctgaaggccgaggataccgccgtgtactattgcgcaaggggattcccactgctgaggcacggcgcctttgactattggggccagggcaccctggtgacagtgagcagcggcggcggcggcagcggcggcggcggcagcggcggcggcggctccgatatccagatgacccagtccccatctagcctgtctgccagcgtgggcgacagggtgaccatcacatgtagagcctctcagaacgtggatacaaatgtggcatggtaccagcagaagccaggacaggcccctaagtccctgatctattccgcctcttaccggtatagcgatgtgccatcccgcttcagcggatccgcctctggcaccgactttaccctgacaatctcctctgtgcagagcgaggattttgccacatactattgccagcagtacaactcatacccttacaccttcggaggaggcacaaaactggaaatcaagtcaggcggcggcggctctgaggtgcagctggtggagagcggcggcggcctggtgcagcccggcggctccctgaagctgtcttgtgccgccagcggcttcaccttcaacaagtatgccatgaattgggtgaggcaggcacctggcaagggcctggagtgggtggccaggatcagaagcaagtacaacaattatgccacctactatgccgactccgtgaaggataggttcacaatctccagagacgattctaagaataccgcctacctgcagatgaacaatctgaagacagaggataccgccgtgtactattgcgtgagacacggcaactttggcaattcttacatcagctattgggcctactggggccagggcacactggtgaccgtgagcagcggcggcggcggcagcggcggcggcggcagcggcggcggcggcagccagacagtggtgacccaggagccaagcctgaccgtgtcccctggcggcaccgtgacactgacctgtggatctagcacaggagcagtgacctccggaaactaccctaattgggtgcagcagaagccaggacaggcccctaggggcctgatcggcggaacaaagttcctggccccaggcacccccgccagattttctggcagcctgctgggcggcaaggccgccctgaccctgtccggagtgcagccagaggacgaggccgagtactattgcgtgctgtggtacagcaatcgctgggtcttcggagggggaaccaaactgactgtcctgtgataa

SEQ ID NO: 40 - protein, 516 aa, Artificial Sequence
  Chemically Synthesized, PSMADBiTE, operably linked to IgE leader
MDWTWILFLVAAATRVHSQVQLVESGGGLVKPGESLRLSCAASGFTFSDYYMYWVRQAPGKGLEWVASISDGGSNTYYSDIIKGRFTISRDNAKNSLYLQMNSLKAEDTAVYYCARGFPLLRHGAFDYWGQGTLVTVSSGGGGSGGGGSGGGGSDIQMTQSPSSLSASVGDRVTITCRASQNVDTNVAWYQQKPGQAPKSLIYSASYRYSDVPSRFSGSASGTDFTLTISSVQSEDFATYYCQQYNSYPYTFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 41 - DNA, 1572 bp, Artificial Sequence
  Chemically Synthesized, PSMADBiTE, operably linked to IgE leader and His tag
atggattggacatggattctgtttctggtcgccgccgctacccgcgtgcatagtcaggtgcagctggtcgaatctggcggcggcctggtcaagcccggagagagcctgagactgagttgtgcagcaagcgggtttactttctccgactactatatgtactgggtgaggcaggcacctggcaagggcctggagtgggtggcaagcatctccgacggcggatctaacacctactatagcgatatcatcaagggcaggttcacaatctccagagacaacgccaagaacagcctgtacctgcagatgaatagcctgaaggccgaggataccgccgtgtactattgcgcaaggggattcccactgctgaggcacggcgcctttgactattggggccagggcaccctggtgacagtgagcagcggcggcggcggcagcggcggcggcggcagcggcggcggcggctccgatatccagatgacccagtccccatctagcctgtctgccagcgtgggcgacagggtgaccatcacatgtagagcctctcagaacgtggatacaaatgtggcatggtaccagcagaagccaggacaggcccctaagtccctgatctattccgcctcttaccggtatagcgatgtgccatcccgcttcagcggatccgcctctggcaccgactttaccctgacaatctcctctgtgcagagcgaggattttgccacatactattgccagcagtacaactcatacccttacaccttcggaggaggcacaaaactggaaatcaagtcaggcggcggcggctctgaggtgcagctggtggagagcggcggcggcctggtgcagcccggcggctccctgaagctgtcttgtgccgccagcggcttcaccttcaacaagtatgccatgaattgggtgaggcaggcacctggcaagggcctggagtgggtggccaggatcagaagcaagtacaacaattatgccacctactatgccgactccgtgaaggataggttcacaatctccagagacgattctaagaataccgcctacctgcagatgaacaatctgaagacagaggataccgccgtgtactattgcgtgagacacggcaactttggcaattcttacatcagctattgggcctactggggccagggcacactggtgaccgtgagcagcggcggcggcggcagcggcggcggcggcagcggcggcggcggcagccagacagtggtgacccaggagccaagcctgaccgtgtcccctggcggcaccgtgacactgacctgtggatctagcacaggagcagtgacctccggaaactaccctaattgggtgcagcagaagccaggacaggcccctaggggcctgatcggcggaacaaagttcctggccccaggcacccccgccagattttctggcagcctgctgggcggcaaggccgccctgaccctgtccggagtgcagccagaggacgaggccgagtactattgcgtgctgtggtacagcaatcgctgggtcttcggagggggaaccaaactgactgtcctgcaccaccaccaccaccattgataa

SEQ ID NO: 42 - protein, 522 aa, Artificial Sequence
  Chemically Synthesized, PSMADBiTE, operably linked to IgE leader and His tag
MDWTWILFLVAAATRVHSQVQLVESGGGLVKPGESLRLSCAASGFTFSDYYMYWVRQAPGKGLEWVASISDGGSNTYYSDIIKGRFTISRDNAKNSLYLQMNSLKAEDTAVYYCARGFPLLRHGAFDYWGQGTLVTVSSGGGGSGGGGSGGGGSDIQMTQSPSSLSASVGDRVTITCRASQNVDTNVAWYQQKPGQAPKSLIYSASYRYSDVPSRFSGSASGTDFTLTISSVQSEDFATYYCQQYNSYPYTFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 43 - DNA, 1488 bp, Artificial Sequence
  Chemically Synthesized, CD123DBiTE without start, stop or leader sequence
gaagtgcagctggtcgagagtggaggaggactggtgcagccaggcggctctctgaggctgagctgcgcagcatccggcttcaccttcaacacctacgcaatgaattgggtgcgccaggcaccaggcaagggcctggagtgggtggccaggatcagaagcaagtacaacaattatgccacatactatgccgcctctgtgaagggccggttcaccatcagccgcgacgattccaagaactctctgtatctgcagatgaacagcctgaagacagaggacaccgccgtgtactattgcgcccggcacggcaacttcggcaatagctacgtgtcctggtttgcctattggggccagggcacactggtgaccgtgagcagcggcggcggcggcagcggcggcggcggcagcggcggcggcggcagccagacagtggtgacccaggagccctctctgacagtgagccctggcggcaccgtgacactgacctgtcggtctagcaccggcgccgtgaccacaagcaactacgccaattgggtgcagcagaagccaggacaggcccccaggggcctgatcggcggaacaaacaagagggcaccaggaacccctgcacgcttctccggctctctgctgggcggcaaggccgccctgaccctgtctggagtgcagcccgaggatgaggccgagtactattgcgccctgtggtactccaatctgtgggtgtttggcggcggaacaaagctgaccgtgctgggcggcggcggcagcgaggtgcagctggtgcagtccggcgccgaggtgaagaagcctggcgagtccctgaagatctcttgtaagggctctggctacagcttcacatcctattggatcagctgggtgaggcagatgcctggcaagggcctggaatggatgggcatcatcgaccctagcgactccgataccagatactctccaagctttcagggccaggtgacaatctccgccgataagtccatctctaccgcctatctgcagtggtcctctctgaaggcctccgacacagccatgtactattgcgcacgcggcgatggatctaccgacctggattactggggacagggcacactggtcaccgtgagcagcggcggcggcggcagcggcggcggcggcagcggcggcggcggctctgagatcgtgctgacacagagcccaggcaccctgagcctgtccccaggagagagggccaccctgtcctgtagagcctctcagagcgtgtctagctcctacctggcctggtatcagcagaagcctggccaggccccaaggctgctgatctacggagcatctagcagggcaacaggcatccccgacagattctccggatctggaagcggaaccgacttcaccctgaccatcagcagactggagcctgaggacttcgccgtgtactattgtcagcaggattatggctttccttggaccttcggacagggcacaaaagtcgagattaagaga

SEQ ID NO: 44 - protein, 496 aa, Artificial Sequence
  Chemically Synthesized, CD123DBiTE without start or leader sequence
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSSGGGGSGGGGSGGGGSQTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSEVQLVQSGAEVKKPGESLKISCKGSGYSFTSYWISWVRQMPGKGLEWMGIIDPSDSDTRYSPSFQGQVTISADKSISTAYLQWSSLKASDTAMYYCARGDGSTDLDYWGQGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQDYGFPWTFGQGTKVEIKR

SEQ ID NO: 45 - DNA, 1548 bp, Artificial Sequence
  Chemically Synthesized, CD123DBiTE, operably linked to IgE leader
atggattggacttggattctgtttctggtcgccgccgcaactagagtgcattctgaagtgcagctggtcgagagtggaggaggactggtgcagccaggcggctctctgaggctgagctgcgcagcatccggcttcaccttcaacacctacgcaatgaattgggtgcgccaggcaccaggcaagggcctggagtgggtggccaggatcagaagcaagtacaacaattatgccacatactatgccgcctctgtgaagggccggttcaccatcagccgcgacgattccaagaactctctgtatctgcagatgaacagcctgaagacagaggacaccgccgtgtactattgcgcccggcacggcaacttcggcaatagctacgtgtcctggtttgcctattggggccagggcacactggtgaccgtgagcagcggcggcggcggcagcggcggcggcggcagcggcggcggcggcagccagacagtggtgacccaggagccctctctgacagtgagccctggcggcaccgtgacactgacctgtcggtctagcaccggcgccgtgaccacaagcaactacgccaattgggtgcagcagaagccaggacaggcccccaggggcctgatcggcggaacaaacaagagggcaccaggaacccctgcacgcttctccggctctctgctgggcggcaaggccgccctgaccctgtctggagtgcagcccgaggatgaggccgagtactattgcgccctgtggtactccaatctgtgggtgtttggcggcggaacaaagctgaccgtgctgggcggcggcggcagcgaggtgcagctggtgcagtccggcgccgaggtgaagaagcctggcgagtccctgaagatctcttgtaagggctctggctacagcttcacatcctattggatcagctgggtgaggcagatgcctggcaagggcctggaatggatgggcatcatcgaccctagcgactccgataccagatactctccaagctttcagggccaggtgacaatctccgccgataagtccatctctaccgcctatctgcagtggtcctctctgaaggcctccgacacagccatgtactattgcgcacgcggcgatggatctaccgacctggattactggggacagggcacactggtcaccgtgagcagcggcggcggcggcagcggcggcggcggcagcggcggcggcggctctgagatcgtgctgacacagagcccaggcaccctgagcctgtccccaggagagagggccaccctgtcctgtagagcctctcagagcgtgtctagctcctacctggcctggtatcagcagaagcctggccaggccccaaggctgctgatctacggagcatctagcagggcaacaggcatccccgacagattctccggatctggaagcggaaccgacttcaccctgaccatcagcagactggagcctgaggacttcgccgtgtactattgtcagcaggattatggctttccttggaccttcggacagggcacaaaagtcgagattaagagatgataa

SEQ ID NO: 46 - protein, 514 aa, Artificial Sequence
  Chemically Synthesized, CD123DBiTE, operably linked to IgE leader
MDWTWILFLVAAATRVHSEVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSSGGGGSGGGGSGGGGSQTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSEVQLVQSGAEVKKPGESLKISCKGSGYSFTSYWISWVRQMPGKGLEWMGIIDPSDSDTRYSPSFQGQVTISADKSISTAYLQWSSLKASDTAMYYCARGDGSTDLDYWGQGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQDYGFPWTFGQGTKVEIKR

SEQ ID NO: 47 - DNA, 1566 bp, Artificial Sequence
  Chemically Synthesized, CD123DBiTE, operably linked to IgE leader and His tag
atggattggacttggattctgtttctggtcgccgccgcaactagagtgcattctgaagtgcagctggtcgagagtggaggaggactggtgcagccaggcggctctctgaggctgagctgcgcagcatccggcttcaccttcaacacctacgcaatgaattgggtgcgccaggcaccaggcaagggcctggagtgggtggccaggatcagaagcaagtacaacaattatgccacatactatgccgcctctgtgaagggccggttcaccatcagccgcgacgattccaagaactctctgtatctgcagatgaacagcctgaagacagaggacaccgccgtgtactattgcgcccggcacggcaacttcggcaatagctacgtgtcctggtttgcctattggggccagggcacactggtgaccgtgagcagcggcggcggcggcagcggcggcggcggcagcggcggcggcggcagccagacagtggtgacccaggagccctctctgacagtgagccctggcggcaccgtgacactgacctgtcggtctagcaccggcgccgtgaccacaagcaactacgccaattgggtgcagcagaagccaggacaggcccccaggggcctgatcggcggaacaaacaagagggcaccaggaacccctgcacgcttctccggctctctgctgggcggcaaggccgccctgaccctgtctggagtgcagcccgaggatgaggccgagtactattgcgccctgtggtactccaatctgtgggtgtttggcggcggaacaaagctgaccgtgctgggcggcggcggcagcgaggtgcagctggtgcagtccggcgccgaggtgaagaagcctggcgagtccctgaagatctcttgtaagggctctggctacagcttcacatcctattggatcagctgggtgaggcagatgcctggcaagggcctggaatggatgggcatcatcgaccctagcgactccgataccagatactctccaagctttcagggccaggtgacaatctccgccgataagtccatctctaccgcctatctgcagtggtcctctctgaaggcctccgacacagccatgtactattgcgcacgcggcgatggatctaccgacctggattactggggacagggcacactggtcaccgtgagcagcggcggcggcggcagcggcggcggcggcagcggcggcggcggctctgagatcgtgctgacacagagcccaggcaccctgagcctgtccccaggagagagggccaccctgtcctgtagagcctctcagagcgtgtctagctcctacctggcctggtatcagcagaagcctggccaggccccaaggctgctgatctacggagcatctagcagggcaacaggcatccccgacagattctccggatctggaagcggaaccgacttcaccctgaccatcagcagactggagcctgaggacttcgccgtgtactattgtcagcaggattatggctttccttggaccttcggacagggcacaaaagtcgagattaagagacaccatcaccaccaccactgataa

SEQ ID NO: 48 - protein, 520 aa, Artificial Sequence
  Chemically Synthesized, CD123DBiTE, operably linked to IgE leader and His tag
MDWTWILFLVAAATRVHSEVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSSGGGGSGGGGSGGGGSQTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSEVQLVQSGAEVKKPGESLKISCKGSGYSFTSYWISWVRQMPGKGLEWMGIIDPSDSDTRYSPSFQGQVTISADKSISTAYLQWSSLKASDTAMYYCARGDGSTDLDYWGQGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQDYGFPWTFGQGTKVEIKRHHHHHH

SEQ ID NO: 49 - DNA, 1485 bp, Artificial Sequence
  Chemically synthesized, nucleotide sequence of HER2DBiTE, without start or stop codons or IgE leader
gatattcagatgactcagagcccttcctccctgagcgcctccgtcggcgatcgggtgaccatcacttgtaaggcaagccaggatgtgagcatcggcgtcgcatggtaccagcagaagcccggcaaagccccaaagctgctgatctattccgcaagttatcggtacaccggcgtcccctcacgcttcagtggatctgggtctgggacagacttcactctgaccattagctccctgcagccagaagattttgccacatactactgtcagcagtactatatctatccttacactttcggacagggaacaaaagtggagatcaagcgcggcgggggagggagcggaggagggggctctggaggcggcgggagcgaggtgcagctggtcgagtctgggggaggcctggtgcagcccggagggtccctgaggctgtcctgtgctgcaagcggctttactttcacagactacaccatggactgggtcagacaggctcccgggaaggggctggaatgggtggccgatgtgaaccctaactctggcggcagtatctacaaccagcggtttaaggggagattcacactgtcagtggacaggagcaaaaatacactgtacctgcagatgaacagcctgagggcagaagatacagcagtgtactactgtgccagaaatctgggacccagtttttatttcgattactgggggcagggaacactggtgacagtgtcatccggcggcgggggctctgaagtccagctggtggagagcggaggaggactggtccagcctggcggaagcctgcggctgtcctgcgctgccagcggatatagttttaccggctacactatgaactgggtgcgccaggcacccggaaaaggactggagtgggtcgctctgatcaacccctataagggagtctcaacttacaatcagaagtttaaagatcgcttcactatcagtgtggacaagtccaagaatacagcctatctgcagatgaactccctgcgcgccgaagatactgccgtctactattgcgcacggtcaggatattacggagacagtgactggtatttcgatgtctggggacaggggacactggtgacagtctcctctggcgggggaggctccgggggaggaggatcaggaggcggcggatctgacattcagatgactcagtcacctagtagcctgtctgctagcgtgggagacagggtcactattacctgcagggccagccaggatatccgcaattacctgaactggtatcagcagaaaccaggcaaagcccccaaactgctgatctactatacctcccggctggaatcaggggtcccttcaaggttctcaggctccggaagtggcaccgattacactctgacaattagctcactgcagccagaagactttgctacctactactgccagcaggggaatactctgccttggaccttcggccagggaactaaagtggagattaagtcatcctgataa

SEQ ID NO: 50 - protein, 493 aa, Artificial Sequence
  Chemically synthesized, HER2DBiTE, without start or IgE leader
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRGGGGSGGGGSGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSS

SEQ ID NO: 51 - DNA, 1539 bp, Artificial Sequence
  Chemically synthesized, HER2DBiTE, with IgE leader
atggactggacttggattctgttcctggtcgcagcagccacacgggtgcattccgatattcagatgactcagagcccttcctccctgagcgcctccgtcggcgatcgggtgaccatcacttgtaaggcaagccaggatgtgagcatcggcgtcgcatggtaccagcagaagcccggcaaagccccaaagctgctgatctattccgcaagttatcggtacaccggcgtcccctcacgcttcagtggatctgggtctgggacagacttcactctgaccattagctccctgcagccagaagattttgccacatactactgtcagcagtactatatctatccttacactttcggacagggaacaaaagtggagatcaagcgcggcgggggagggagcggaggagggggctctggaggcggcgggagcgaggtgcagctggtcgagtctgggggaggcctggtgcagcccggagggtccctgaggctgtcctgtgctgcaagcggctttactttcacagactacaccatggactgggtcagacaggctcccgggaaggggctggaatgggtggccgatgtgaaccctaactctggcggcagtatctacaaccagcggtttaaggggagattcacactgtcagtggacaggagcaaaaatacactgtacctgcagatgaacagcctgagggcagaagatacagcagtgtactactgtgccagaaatctgggacccagtttttatttcgattactgggggcagggaacactggtgacagtgtcatccggcggcgggggctctgaagtccagctggtggagagcggaggaggactggtccagcctggcggaagcctgcggctgtcctgcgctgccagcggatatagttttaccggctacactatgaactgggtgcgccaggcacccggaaaaggactggagtgggtcgctctgatcaacccctataagggagtctcaacttacaatcagaagtttaaagatcgcttcactatcagtgtggacaagtccaagaatacagcctatctgcagatgaactccctgcgcgccgaagatactgccgtctactattgcgcacggtcaggatattacggagacagtgactggtatttcgatgtctggggacaggggacactggtgacagtctcctctggcgggggaggctccgggggaggaggatcaggaggcggcggatctgacattcagatgactcagtcacctagtagcctgtctgctagcgtgggagacagggtcactattacctgcagggccagccaggatatccgcaattacctgaactggtatcagcagaaaccaggcaaagcccccaaactgctgatctactatacctcccggctggaatcaggggtcccttcaaggttctcaggctccggaagtggcaccgattacactctgacaattagctcactgcagccagaagactttgctacctactactgccagcaggggaatactctgccttggaccttcggccagggaactaaagtggagattaagtcatcctgataa

SEQ ID NO: 52 - protein, 511 aa, Artificial Sequence
  Chemically synthesized, HER2DBiTE, with IgE leader
MDWTWILFLVAAATRVHSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRGGGGSGGGGSGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSS

SEQ ID NO: 53 - DNA, 1560 bp, Artificial Sequence
  Chemically synthesized, CD3xHer2 HER2DBiTE with His6 tag
atggactggacttggagaatcctgttcctggtcgctgccgccactggaactcacgcccagattgtgctgactcagagccctgctattatgtccgcctctcccggcgagaaggtgaccatgacctgttctgcctccagctccgtgtcctatgacggcgatagctacatgaactggtaccagcagaagtctggcacctctcccaagagatggatctacgataccagcaagctggccagcggcgtgcccgcccactttcggggcagcggctccggcaccagctactctctgacaatctccggcatggaggccgaggatgccgccacctactactgccagcagtggtcctctaatcctttcaccttcggctctggcacaaagctggagatcaatcgcggcggcggcggctccggcggcggcggcagcggcggcggcggctcccaggtgcagctgcagcagtctggcgccgagctggcccgccctggcgcctctgtgaagatgtcttgtaaggcctccggctacaccttcaccagatataccatgcactgggtgaagcagcgcccaggccagggcctggagtggatcggctatatcaatccatctagaggctacaccaattacaaccagaagttcaaggataaggccaccctgaccacagacaagtcctcctctacagcctacatgcagctgtcctctctgacatccgaggattccgccgtgtactattgtgccagatattacgacgatcactactgtctggactattggggccagggcacaaccctgacagtgtccagcggcggcggcggctctgaggtgcagctggtggagtctggcggcggcctggtgcagcccggcggctctctgagactgtcttgtgccgcctctggctttaccttcacagattataccatggattgggtgagacaggcccctggcaagggcctggagtgggtggccgatgtgaaccccaatagcggcggctccatctacaatcagaggtttaagggccgctttacactgtctgtggaccggtctaagaacaccctgtacctgcagatgaattctctgagagccgaggacaccgccgtgtattactgtgccaggaatctgggcccttctttctatttcgactactggggccagggcacactggtgaccgtgtcctccggcggcggcggcagcggcggcggcggctctggcggcggcggcagcgacatccagatgacccagtccccaagctccctgtctgcctccgtgggcgatcgcgtgaccatcacatgcaaggcctcccaggacgtgtctatcggcgtggcctggtaccagcagaagcctggcaaggccccaaagctgctgatctactctgcctcctatagatacaccggcgtgccctctagattctccggctctggctctggcaccgacttcacactgaccatctcttctctgcagcccgaggacttcgccacctactattgtcagcagtactacatctacccttacaccttcggacaggggacaaaagtggaaatcaagagacatcaccatcaccatcattgataa

SEQ ID NO: 54 - protein, 518 aa, Artificial Sequence
  Chemically synthesized, CD3xHer2 HER2DBiTE with His6 tag
MDWTWRILFLVAAATGTHAQIVLTQSPAIMSASPGEKVTMTCSASSSVSYDGDSYMNWYQQKSGTSPKRWIYDTSKLASGVPAHFRGSGSGTSYSLTISGMEAEDAATYYCQQWSSNPFTFGSGTKLEINRGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGGGSGGGGSGGGGSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRHHHHHH

SEQ ID NO: 55 - DNA, 1539 bp, Artificial Sequence
  Chemically synthesized, CD3xHer2 HER2DBiTE with IgE leader
atggattggacctggattctgtttctggtggcggcggcgacccgcgtgcatagccagattgtgctgactcagagccctgctattatgtccgcctctcccggcgagaaggtgaccatgacctgttctgcctccagctccgtgtcctatgacggcgatagctacatgaactggtaccagcagaagtctggcacctctcccaagagatggatctacgataccagcaagctggccagcggcgtgcccgcccactttcggggcagcggctccggcaccagctactctctgacaatctccggcatggaggccgaggatgccgccacctactactgccagcagtggtcctctaatcctttcaccttcggctctggcacaaagctggagatcaatcgcggcggcggcggctccggcggcggcggcagcggcggcggcggctcccaggtgcagctgcagcagtctggcgccgagctggcccgccctggcgcctctgtgaagatgtcttgtaaggcctccggctacaccttcaccagatataccatgcactgggtgaagcagcgcccaggccagggcctggagtggatcggctatatcaatccatctagaggctacaccaattacaaccagaagttcaaggataaggccaccctgaccacagacaagtcctcctctacagcctacatgcagctgtcctctctgacatccgaggattccgccgtgtactattgtgccagatattacgacgatcactactgtctggactattggggccagggcacaaccctgacagtgtccagcggcggcggcggctctgaggtgcagctggtggagtctggcggcggcctggtgcagcccggcggctctctgagactgtcttgtgccgcctctggctttaccttcacagattataccatggattgggtgagacaggcccctggcaagggcctggagtgggtggccgatgtgaaccccaatagcggcggctccatctacaatcagaggtttaagggccgctttacactgtctgtggaccggtctaagaacaccctgtacctgcagatgaattctctgagagccgaggacaccgccgtgtattactgtgccaggaatctgggcccttctttctatttcgactactggggccagggcacactggtgaccgtgtcctccggcggcggcggcagcggcggcggcggctctggcggcggcggcagcgacatccagatgacccagtccccaagctccctgtctgcctccgtgggcgatcgcgtgaccatcacatgcaaggcctcccaggacgtgtctatcggcgtggcctggtaccagcagaagcctggcaaggccccaaagctgctgatctactctgcctcctatagatacaccggcgtgccctctagattctccggctctggctctggcaccgacttcacactgaccatctcttctctgcagcccgaggacttcgccacctactattgtcagcagtactacatctacccttacaccttcggacaggggacaaaagtggaaatcaagagatgataa

SEQ ID NO: 56 - protein, 511 aa, Artificial Sequence
  Chemically synthesized, CD3xHer2 HER2DBiTE with IgE leader
MDWTWILFLVAAATRVHSQIVLTQSPAIMSASPGEKVTMTCSASSSVSYDGDSYMNWYQQKSGTSPKRWIYDTSKLASGVPAHFRGSGSGTSYSLTISGMEAEDAATYYCQQWSSNPFTFGSGTKLEINRGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGGGSGGGGSGGGGSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKR

SEQ ID NO: 57 - DNA, 1542 bp, Artificial Sequence
  Chemically synthesized, HER2DBiTE-L
atggattggacctggatcctgtttctggtggccgctgccacaagagtgcacagcgaagtgcagctggtggaatctggcggaggactggttcaacctggcggctctctgagactgtcttgtgccgccagcggcttcaccttcaacacctacgccatgaactgggtccgacaggcccctggcaaaggccttgaatgggtcgccagaatcagaagcaagtacaacaattacgccacctactacgccgcctccgtgaagggcagattcaccatcagcagggacgacagcaagaacagcctgtacctgcagatgaactccctgaaaaccgaggacaccgccgtgtactactgtgccagacacggcaacttcggcaacagctatgtgtcttggtttgcctactggggccagggcacactggtcacagtttctagcggaggcggaggttctggcggcggaggaagtggtggcggaggctctcaaacagtggtcacccaagagcctagcctgaccgtttctcctggcggaaccgtgacactgacctgtagatcttctacaggcgccgtgaccaccagcaactacgccaattgggtgcagcagaagccaggccaggctcctagaggactgatcggaggcaccaacaagagagcccctggaacaccagccagattctctggctctctgctcggaggaaaggccgctctgacactgagcggagtgcagcctgaagatgaggccgagtactattgcgccctgtggtacagcaacctgtgggttttcggcggaggcacaaagctgacagtgcttggaggtggcggatccgaggttcagctggttgaaagtggcggtggcttggtgcaaccaggcggaagcctcagacttagctgtgccgcctctggctttacctttaccgactacacaatggattgggttcgacaagctccaggcaagggactcgagtgggtcgccgatgtgaatcctaatagcggcggcagcatctacaaccagcggttcaaaggccggttcaccctgagcgtggacagatccaagaatactctgtatctccagatgaatagcctgcgcgccgaggatacagctgtgtattactgcgcccggaatctgggccccagcttctactttgattattggggacagggaaccctcgtgaccgtgtctagtggcggaggtggaagtggcggaggcggttcaggtggcggaggatctgatatccagatgacacagagccccagcagcctgtctgcctctgtgggagacagagtgaccatcacatgcaaggccagccaggacgtgtccattggcgtggcatggtatcagcagaaacccggcaaggcccctaagctgctgatctacagcgccagctacagatacaccggcgtgccctctagattttccggcagcggcagcggaaccgactttaccctgacaatcagctccctgcagccagaggacttcgccacctattactgccagcagtactacatctacccctacaccttcggacagggcaccaaggtggaaatcaagcgg

SEQ ID NO: 58 - protein, 514 aa, Artificial Sequence
  Chemically synthesized, HER2DBiTE-L
MDWTWILFLVAAATRVHSEVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSSGGGGSGGGGSGGGGSQTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGGGSGGGGSGGGGSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKR

SEQ ID NO: 59 - DNA, 1560 bp, Artificial Sequence
  Chemically synthesized, HER2DBiTE-L with His tag
atggattggacctggatcctgtttctggtggccgctgccacaagagtgcacagcgaagtgcagctggtggaatctggcggaggactggttcaacctggcggctctctgagactgtcttgtgccgccagcggcttcaccttcaacacctacgccatgaactgggtccgacaggcccctggcaaaggccttgaatgggtcgccagaatcagaagcaagtacaacaattacgccacctactacgccgcctccgtgaagggcagattcaccatcagcagggacgacagcaagaacagcctgtacctgcagatgaactccctgaaaaccgaggacaccgccgtgtactactgtgccagacacggcaacttcggcaacagctatgtgtcttggtttgcctactggggccagggcacactggtcacagtttctagcggaggcggaggttctggcggcggaggaagtggtggcggaggctctcaaacagtggtcacccaagagcctagcctgaccgtttctcctggcggaaccgtgacactgacctgtagatcttctacaggcgccgtgaccaccagcaactacgccaattgggtgcagcagaagccaggccaggctcctagaggactgatcggaggcaccaacaagagagcccctggaacaccagccagattctctggctctctgctcggaggaaaggccgctctgacactgagcggagtgcagcctgaagatgaggccgagtactattgcgccctgtggtacagcaacctgtgggttttcggcggaggcacaaagctgacagtgcttggaggtggcggatccgaggttcagctggttgaaagtggcggtggcttggtgcaaccaggcggaagcctcagacttagctgtgccgcctctggctttacctttaccgactacacaatggattgggttcgacaagctccaggcaagggactcgagtgggtcgccgatgtgaatcctaatagcggcggcagcatctacaaccagcggttcaaaggccggttcaccctgagcgtggacagatccaagaatactctgtatctccagatgaatagcctgcgcgccgaggatacagctgtgtattactgcgcccggaatctgggccccagcttctactttgattattggggacagggaaccctcgtgaccgtgtctagtggcggaggtggaagtggcggaggcggttcaggtggcggaggatctgatatccagatgacacagagccccagcagcctgtctgcctctgtgggagacagagtgaccatcacatgcaaggccagccaggacgtgtccattggcgtggcatggtatcagcagaaacccggcaaggcccctaagctgctgatctacagcgccagctacagatacaccggcgtgccctctagattttccggcagcggcagcggaaccgactttaccctgacaatcagctccctgcagccagaggacttcgccacctattactgccagcagtactacatctacccctacaccttcggacagggcaccaaggtggaaatcaagcggcaccaccaccatcaccac

SEQ ID NO: 60 - protein, 520 aa, Artificial Sequence
  Chemically synthesized, HER2DBiTE-L with His tag
MDWTWILFLVAAATRVHSEVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSSGGGGSGGGGSGGGGSQTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGGGSGGGGSGGGGSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRHHHHHH

SEQ ID NO: 61 - DNA, 2190 bp, Artificial Sequence
  Chemically synthesized, human Her2DMAb
atggattggacatggattctgtttctggtcgccgccgccacaagagtgcattccgaagtgcagctggtcgagtcagggggaggactggtgcagcccggcggaagcctgcgactgtcctgcgccgcttctggcttcacttttaccgactacaccatggattgggtgcgccaggcacctgggaagggactggaatgggtcgctgatgtgaacccaaatagtgggggctcaatctacaaccagcgattcaaaggcaggtttaccctgagtgtggacagatcaaagaacacactgtatctgcagatgaatagcctgagggcagaggataccgccgtgtactattgcgccagaaatctgggaccatccttctactttgactattggggacaggggactctggtcaccgtgagctccgcctcaacaaaaggccccagcgtgttccccctggctccttctagtaagtctacaagtggagggactgcagccctgggatgtctggtgaaggactacttccctgagccagtcaccgtgagctggaactccggcgctctgacttctggagtccacacctttcccgcagtgctgcagtcaagcgggctgtactccctgtcctctgtggtcaccgtccctagttcaagcctgggcacacagacttatatctgcaacgtgaatcacaaaccatctaatacaaaggtcgacaagaaagtggaacccaaaagctgtgataagacccatacatgccctccctgtccagctcctgagctgctgggcggaccaagcgtgttcctgtttccacccaagcctaaagataccctgatgatttcccggaccccagaagtcacatgcgtggtcgtggacgtgtctcacgaggaccccgaagtcaagtttaactggtacgtggacggcgtcgaggtgcataatgccaagacaaaaccacgcgaggaacagtacaactccacttatcgagtcgtgtctgtcctgaccgtgctgcatcaggattggctgaacgggaaggagtataagtgcaaagtgagcaacaaggccctgccagctcccatcgagaagaccatttccaaggcaaaaggccagccacgggaaccccaggtgtacacactgcctccatctcgcgatgagctgaccaaaaaccaggtcagtctgacatgtctggtgaagggcttctatccctcagacatcgccgtggagtgggaatccaatggacagcctgaaaacaattacaagaccacaccccctgtgctggactctgatggaagtttctttctgtatagtaaactgactgtggataagtcaaggtggcagcaggggaacgtcttttcatgcagcgtgatgcacgaggccctgcacaatcattacacacagaaatccctgtctctgagtcctggaaaacgggggcgcaagaggagatcaggcagcggagcaacaaacttcagcctgctgaagcaggcaggggacgtggaggaaaatcctggcccaatggattggacttggattctgttcctggtcgctgcagccactagggtgcatagcgacattcagatgacccagtccccctcctctctgtccgcctctgtcggcgacagagtgactatcacctgtaaggctagccaggatgtctccattggagtggcatggtaccagcagaagcctgggaaagctccaaagctgctgatctacagtgcatcatacagatatacaggagtgccttcccggttcagcgggtccggctctggaactgactttacactgactatcagttcactgcagcctgaggatttcgctacctactattgccagcagtactatatctacccatatacatttgggcagggcactaaagtggaaatcaagcggactgtcgctgcacctagcgtgttcatctttccacccagtgatgagcagctgaagtctggaaccgccagtgtggtgtgcctgctgaacaatttctacccacgcgaagccaaagtccagtggaaggtggacaacgctctgcagtcagggaatagccaggagtccgtgacagaacaggactctaaagatagtacttattcactgagctccaccctgacactgtccaaggcagactacgagaagcacaaagtgtatgcctgcgaggtcactcaccaggggctgtcttcacccgtcactaagtcctttaatagaggggaatgttgataa

SEQ ID NO: 62 - protein, 728 aa, Artificial Sequence
  Chemically synthesized, human Her2DMAb
MDWTWILFLVAAATRVHSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMDWTWILFLVAAATRVHSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - DNA, 2187 bp, Artificial Sequence
  Chemically synthesized, mouse Her2DMAb
atggattggacatggatcctgttcctggtggccgctgccaccagggtgcacagcgaggtgcagctggtggagtccggcggaggcctggtgcagccaggaggctctctgaggctgagctgcgctgcctccggcttcacctttacagactacaccatggattgggtgagacaggctccaggaaagggcctggagtgggtggctgacgtgaaccctaactccggaggctctatctacaaccagaggttcaagggcaggttcaccctgtctgtggaccgcagcaagaacaccctgtacctgcagatgaactccctgcgggccgaggatacagccgtgtactactgtgcccgcaacctgggaccttctttctactttgactactggggacagggcacactggtgaccgtgagctccgccaagaccacagctccatccgtgtacccactggctcccgtgtgcggcgacaccacaggatctagcgtgaccctgggctgtctggtgaagggatacttccccgagcctgtgaccctgacatggaacagcggctccctgtcctctggagtgcacacatttcctgccgtgctgcagagcgatctgtacaccctgagctcctctgtgaccgtgacaagctccacatggccttctcagagcatcacctgcaacgtggcccacccagcttctagcacaaaggtggacaagaagatcgagcctcggggcccaaccatcaagccttgtcccccttgcaagtgtccagctccaaacctgctgggaggcccatccgtgttcatctttccacccaagatcaaggatgtgctgatgatctccctgtctccaatcgtgacctgcgtggtggtggacgtgtccgaggacgatcccgatgtgcagatctcttggttcgtgaacaacgtggaggtgcacacagcccagacccagacacacagggaggactacaactccaccctgagagtggtgtctgctctgccaatccagcaccaggactggatgagcggcaaggagtttaagtgcaaggtgaacaacaaggatctgcctgccccaatcgagaggacaatctctaagccaaagggaagcgtgagagctccccaggtgtacgtgctgcctccacccgaggaggagatgacaaagaagcaggtgaccctgacatgtatggtgaccgacttcatgccagaggatatctacgtggagtggacaaacaacggcaagaccgagctgaactacaagaacaccgagcccgtgctggacagcgatggatcctactttatgtactccaagctgcgggtggagaagaagaactgggtggagcgcaacagctactcctgctctgtggtgcacgagggcctgcacaaccaccacaccacaaagagcttctccaggacacccggcaagcggggacgcaagaggagatctggaagcggcgccaccaactttagcctgctgaagcaggctggcgacgtggaggagaacccaggacctatggattggacctggatcctgttcctggtcgctgccgctacacgggtgcactctgacatccagatgacccagagcccatcctctctgtccgcctctgtgggcgaccgcgtgaccatcacctgtaaggcttcccaggacgtgagcatcggagtggcttggtaccagcagaagcccggaaaggcccctaagctgctgatctacagcgcctcctaccggtacacaggagtgccatcccgcttctctggaagcggatccggaaccgactttaccctgacaatcagctccctgcagcccgaggatttcgccacctactactgccagcagtactacatctacccttacacatttggccagggaaccaaggtggagatcaaggccgacgccgctcccacagtgagcatcttccctccatctagcgagcagctgaccagcggaggcgcttccgtggtgtgcttcctgaacaacttttaccctaaggacatcaacgtgaagtggaagatcgatggcagcgagagacagaacggagtgctgaactcctggacagaccaggattctaaggacagcacctactccatgtcctctaccctgacactgaccaaggatgagtacgagaggcacaactcttacacatgcgaggccacccacaagacatctaccagccctatcgtgaagagctttaacagaaacgagtgttgataa

SEQ ID NO: 64 - protein, 727 aa, Artificial Sequence
  Chemically synthesized, mouse Her2DMAb
MDWTWILFLVAAATRVHSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKRGRKRRSGSGATNFSLLKQAGDVEENPGPMDWTWILFLVAAATRVHSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 65 - DNA, 726 bp, Artificial Sequence
  Chemically synthesized, scFv Her2DMAb
gaggtgcagctggtggagtctggcggcggcctggtgcagcccggcggctctctgagactgtcttgtgccgcctctggctttaccttcacagattataccatggattgggtgagacaggcccctggcaagggcctggagtgggtggccgatgtgaaccccaatagcggcggctccatctacaatcagaggtttaagggccgctttacactgtctgtggaccggtctaagaacaccctgtacctgcagatgaattctctgagagccgaggacaccgccgtgtattactgtgccaggaatctgggcccttctttctatttcgactactggggccagggcacactggtgaccgtgtcctccggcggcggcggcagcggcggcggcggctctggcggcggcggcagcgacatccagatgacccagtccccaagctccctgtctgcctccgtgggcgatcgcgtgaccatcacatgcaaggcctcccaggacgtgtctatcggcgtggcctggtaccagcagaagcctggcaaggccccaaagctgctgatctactctgcctcctatagatacaccggcgtgccctctagattctccggctctggctctggcaccgacttcacactgaccatctcttctctgcagcccgaggacttcgccacctactattgtcagcagtactacatctacccttacaccttcggacaggggacaaaagtggaaatcaagaga

SEQ ID NO: 66 - protein, 241 aa, Artificial Sequence
  Chemically synthesized, scFv Her2DMAb
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGGGSGGGGSGGGGSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIK

SEQ ID NO: 67 - DNA, 4493 bp, Artificial Sequence
  Chemically synthesized, pGX93237 full plasmid sequence
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatggactggacttggattctgttcctggtcgcagcagccacacgggtgcattccgatattcagatgactcagagcccttcctccctgagcgcctccgtcggcgatcgggtgaccatcacttgtaaggcaagccaggatgtgagcatcggcgtcgcatggtaccagcagaagcccggcaaagccccaaagctgctgatctattccgcaagttatcggtacaccggcgtcccctcacgcttcagtggatctgggtctgggacagacttcactctgaccattagctccctgcagccagaagattttgccacatactactgtcagcagtactatatctatccttacactttcggacagggaacaaaagtggagatcaagcgcggcgggggagggagcggaggagggggctctggaggcggcgggagcgaggtgcagctggtcgagtctgggggaggcctggtgcagcccggagggtccctgaggctgtcctgtgctgcaagcggctttactttcacagactacaccatggactgggtcagacaggctcccgggaaggggctggaatgggtggccgatgtgaaccctaactctggcggcagtatctacaaccagcggtttaaggggagattcacactgtcagtggacaggagcaaaaatacactgtacctgcagatgaacagcctgagggcagaagatacagcagtgtactactgtgccagaaatctgggacccagtttttatttcgattactgggggcagggaacactggtgacagtgtcatccggcggcgggggctctgaagtccagctggtggagagcggaggaggactggtccagcctggcggaagcctgcggctgtcctgcgctgccagcggatatagttttaccggctacactatgaactgggtgcgccaggcacccggaaaaggactggagtgggtcgctctgatcaacccctataagggagtctcaacttacaatcagaagtttaaagatcgcttcactatcagtgtggacaagtccaagaatacagcctatctgcagatgaactccctgcgcgccgaagatactgccgtctactattgcgcacggtcaggatattacggagacagtgactggtatttcgatgtctggggacaggggacactggtgacagtctcctctggcgggggaggctccgggggaggaggatcaggaggcggcggatctgacattcagatgactcagtcacctagtagcctgtctgctagcgtgggagacagggtcactattacctgcagggccagccaggatatccgcaattacctgaactggtatcagcagaaaccaggcaaagcccccaaactgctgatctactatacctcccggctggaatcaggggtcccttcaaggttctcaggctccggaagtggcaccgattacactctgacaattagctcactgcagccagaagactttgctacctactactgccagcaggggaatactctgccttggaccttcggccagggaactaaagtggagattaagtcatcctgataactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 68 - protein, 15 aa, Artificial Sequence
  Chemically synthesized, G4S linker
GGGGSGGGGSGGGGS

SEQ ID NO: 69 - DNA, 1506 bp, Artificial Sequence
  Chemically Synthesized, EGFRvIII DNA encoded immune cell engager
gatgtcgtgatgacccagagccctgatagcctggccgtgagcctgggagagagggcaaccatcaactgcaagagctcccagagcctgctggactccgatggcaagacatacctgaattggctgcagcagaagcctggccagccccctaagaggctgatctctctggtgagcaagctggactccggagtgccagatcgcttcagcggctccggctctggaaccgactttaccctgacaatctctagcctgcaggccgaggatgtggccgtgtactattgctggcagggcacacacttccccggcacctttggcggcggcacaaaggtggagatcaagggcggcggcggctctggaggaggaggcagcggcggaggaggctccggaggcggcggctctgagatccagctggtgcagagcggagcagaggtgaagaagccaggcgagtctctgagaatcagctgtaagggctccggcttcaacatcgaggactactatatccactgggtgcggcagatgcctggcaagggcctggagtggatgggcagaatcgacccagagaacgatgagacaaagtatggccccatcttccagggccacgtgacaatctctgccgacacaagcatcaataccgtgtacctgcagtggtcctctctgaaggcctccgataccgccatgtactattgcgcctttaggggaggcgtgtattggggacagggaaccacagtgacagtgagctccggaggaggaggcagcgaggtgcagctggtggagagcggcggcggcctggtgcagccaggcggctccctgaagctgtcttgtgccgccagcggcttcacctttaacaagtacgcaatgaattgggtgcgccaggcaccaggcaagggcctggagtgggtggcccggatcagatctaagtacaacaattatgccacatactatgccgacagcgtgaaggataggttcaccatctcccgcgacgattctaagaacacagcctacctgcagatgaacaatctgaagacagaggacaccgccgtgtactattgcgtgaggcacggcaactttggcaattcctacatctcttattgggcctactggggccagggcacactggtgaccgtgtctagcggcggcggcggcagcggcggcggcggctccggaggaggcggctctcagacagtggtgacccaggagccaagcctgaccgtgtcccctggaggcaccgtgacactgacctgtggctcctctacaggagcagtgaccagcggaaactatcctaattgggtgcagcagaagcctggacaggcaccacggggcctgatcggaggaaccaagttcctggcaccaggaacacccgcccggttcagcggctccctgctgggaggcaaggccgccctgaccctgtccggcgtgcagccagaggatgaggccgagtactattgcgtgctgtggtactctaatcggtgggtctttggcgggggaactaaactgactgtcctgtgataa

SEQ ID NO: 70 - protein, 500 aa, Artificial Sequence
  Chemically Synthesized, EGFRvIII DNA encoded immune cell engager
DVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIKGGGGSGGGGSGGGGSGGGGSEIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 71 - DNA, 1560 bp, Artificial Sequence
  Chemically Synthesized, EGFRvIII DNA encoded immune cell engager with IgE leader
atggattggacttggattctgttcctggtggcagccgcaactagagtgcattccgatgtcgtgatgacccagagccctgatagcctggccgtgagcctgggagagagggcaaccatcaactgcaagagctcccagagcctgctggactccgatggcaagacatacctgaattggctgcagcagaagcctggccagccccctaagaggctgatctctctggtgagcaagctggactccggagtgccagatcgcttcagcggctccggctctggaaccgactttaccctgacaatctctagcctgcaggccgaggatgtggccgtgtactattgctggcagggcacacacttccccggcacctttggcggcggcacaaaggtggagatcaagggcggcggcggctctggaggaggaggcagcggcggaggaggctccggaggcggcggctctgagatccagctggtgcagagcggagcagaggtgaagaagccaggcgagtctctgagaatcagctgtaagggctccggcttcaacatcgaggactactatatccactgggtgcggcagatgcctggcaagggcctggagtggatgggcagaatcgacccagagaacgatgagacaaagtatggccccatcttccagggccacgtgacaatctctgccgacacaagcatcaataccgtgtacctgcagtggtcctctctgaaggcctccgataccgccatgtactattgcgcctttaggggaggcgtgtattggggacagggaaccacagtgacagtgagctccggaggaggaggcagcgaggtgcagctggtggagagcggcggcggcctggtgcagccaggcggctccctgaagctgtcttgtgccgccagcggcttcacctttaacaagtacgcaatgaattgggtgcgccaggcaccaggcaagggcctggagtgggtggcccggatcagatctaagtacaacaattatgccacatactatgccgacagcgtgaaggataggttcaccatctcccgcgacgattctaagaacacagcctacctgcagatgaacaatctgaagacagaggacaccgccgtgtactattgcgtgaggcacggcaactttggcaattcctacatctcttattgggcctactggggccagggcacactggtgaccgtgtctagcggcggcggcggcagcggcggcggcggctccggaggaggcggctctcagacagtggtgacccaggagccaagcctgaccgtgtcccctggaggcaccgtgacactgacctgtggctcctctacaggagcagtgaccagcggaaactatcctaattgggtgcagcagaagcctggacaggcaccacggggcctgatcggaggaaccaagttcctggcaccaggaacacccgcccggttcagcggctccctgctgggaggcaaggccgccctgaccctgtccggcgtgcagccagaggatgaggccgagtactattgcgtgctgtggtactctaatcggtgggtctttggcgggggaactaaactgactgtcctgtgataa

SEQ ID NO: 72 - protein, 518 aa, Artificial Sequence
  Chemically Synthesized, EGFRvIII DNA encoded immune cell engager with IgE leader
MDWTWILFLVAAATRVHSDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIKGGGGSGGGGSGGGGSGGGGSEIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 73 - DNA, 1461 bp, Artificial Sequence
  Chemically Synthesized, Her2 DNA encoded immune cell engager
gacatccagatgacccagagccccagcagcctgagcgccagcgtgggcgacagggtgaccatcacctgcaaggccagccaggacgtgagcatcggcgtggcctggtaccagcagaagcccggcaaggcccccaagctgctgatctacagcgccagctacaggtacaccggcgtgcccagcaggttcagcggcagcggcagcggcaccgacttcaccctgaccatcagcagcctgcagcccgaggacttcgccacctactactgccagcagtactacatctacccctacaccttcggccagggcaccaaggtggagatcaagggcggcggcggcagcggcggcggcggcagcggcggcggcggcagcgaggtgcagctggtggagagcggcggcggcctggtgcagcccggcggcagcctgaggctgagctgcgccgccagcggcttcaccttcaccgactacaccatggactgggtgaggcaggcccccggcaagggcctggagtgggtggccgacgtgaaccccaacagcggcggcagcatctacaaccagaggttcaagggcaggttcaccctgagcgtggacaggagcaagaacaccctgtacctgcagatgaacagcctgagggccgaggacaccgccgtgtactactgcgccaggaacctgggccccagcttctacttcgactactggggccagggcaccctggtgaccgtgagcagcggcggcggcggcagccaggtgcagctggtggagagcggcggcggcgtggtgcagcccggcaggagcctgaggctgagctgcgccgccagcggcttcaagttcagcggctacggcatgcactgggtgaggcaggcccccggcaagggcctggagtgggtggccgtgatctggtacgacggcagcaagaagtactacgtggacagcgtgaagggcaggttcaccatcagcagggacaacagcaagaacaccctgtacctgcagatgaacagcctgagggccgaggacaccgccgtgtactactgcgccaggcagatgggctactggcacttcgacctgtggggcaggggcaccctggtgaccgtgagcagcggcggcggcggcagcggcggcggcggcagcggcggcggcggcagcgagatcgtgctgacccagagccccgccaccctgagcctgagccccggcgagagggccaccctgagctgcagggccagccagagcgtgagcagctacctggcctggtaccagcagaagcccggccaggcccccaggctgctgatctacgacgccagcaacagggccaccggcatccccgccaggttcagcggcagcggcagcggcaccgacttcaccctgaccatcagcagcctggagcccgaggacttcgccgtgtactactgccagcagaggagcaactggccccccctgaccttcggcggcggcaccaaggtgatcgagaag

SEQ ID NO: 74 - protein, 493 aa, Artificial Sequence; identical to SEQ ID NO: 50
  Chemically Synthesized, Her2 DNA encoded immune cell engager

SEQ ID NO: 75 - DNA, 1515 bp, Artificial Sequence
  Chemically Synthesized, Her2 DNA encoded immune cell engager with IgE leader
atggactggacctggatcctgttcctggtggccgccgccaccagggtgcacagcgacatccagatgacccagagccccagcagcctgagcgccagcgtgggcgacagggtgaccatcacctgcaaggccagccaggacgtgagcatcggcgtggcctggtaccagcagaagcccggcaaggcccccaagctgctgatctacagcgccagctacaggtacaccggcgtgcccagcaggttcagcggcagcggcagcggcaccgacttcaccctgaccatcagcagcctgcagcccgaggacttcgccacctactactgccagcagtactacatctacccctacaccttcggccagggcaccaaggtggagatcaagggcggcggcggcagcggcggcggcggcagcggcggcggcggcagcgaggtgcagctggtggagagcggcggcggcctggtgcagcccggcggcagcctgaggctgagctgcgccgccagcggcttcaccttcaccgactacaccatggactgggtgaggcaggcccccggcaagggcctggagtgggtggccgacgtgaaccccaacagcggcggcagcatctacaaccagaggttcaagggcaggttcaccctgagcgtggacaggagcaagaacaccctgtacctgcagatgaacagcctgagggccgaggacaccgccgtgtactactgcgccaggaacctgggccccagcttctacttcgactactggggccagggcaccctggtgaccgtgagcagcggcggcggcggcagccaggtgcagctggtggagagcggcggcggcgtggtgcagcccggcaggagcctgaggctgagctgcgccgccagcggcttcaagttcagcggctacggcatgcactgggtgaggcaggcccccggcaagggcctggagtgggtggccgtgatctggtacgacggcagcaagaagtactacgtggacagcgtgaagggcaggttcaccatcagcagggacaacagcaagaacaccctgtacctgcagatgaacagcctgagggccgaggacaccgccgtgtactactgcgccaggcagatgggctactggcacttcgacctgtggggcaggggcaccctggtgaccgtgagcagcggcggcggcggcagcggcggcggcggcagcggcggcggcggcagcgagatcgtgctgacccagagccccgccaccctgagcctgagccccggcgagagggccaccctgagctgcagggccagccagagcgtgagcagctacctggcctggtaccagcagaagcccggccaggcccccaggctgctgatctacgacgccagcaacagggccaccggcatccccgccaggttcagcggcagcggcagcggcaccgacttcaccctgaccatcagcagcctggagcccgaggacttcgccgtgtactactgccagcagaggagcaactggccccccctgaccttcggcggcggcaccaaggtgatcgagaag

SEQ ID NO: 76 - protein, 511 aa, Artificial Sequence; identical to SEQ ID NO: 52
  Chemically Synthesized, Her2 DNA encoded immune cell engager with IgE leader

(19) United States
(12) Patent Application Publication
(10) Pub. No.: US 2022/0098324 A1
(43) Pub. Date: Mar. 31, 2022

(54) DNA-ENCODED BISPECIFIC T-CELL ENGAGERS TARGETING CANCER ANTIGENS AND METHODS OF USE IN CANCER THERAPUTICS

(71) Applicant: THE WISTAR INSTITUTE OF ANATOMY AND BIOLOGY, Philadelphia, PA (US)

(72) Inventors: David Weiner, Merion, PA (US); Alfredo Perales-Puchalt, Philadelphia, PA (US); Kar Muthumani, Cherry Hill, NJ (US); Elizabeth Duperret, Philadelphia, PA (US)

(21) Appl. No.: 17/427,434

(22) PCT Filed: Jan. 30, 2020
(86) PCT No.: PCT/US20/15942

§ 371 Date: Jul. 30, 2021

Related U.S. Application Data
(60) Provisional application No. 62/798,626, filed on Jan. 30, 2019.
(60) Provisional application No. 62/827,265, filed on Apr. 1, 2019.

(51) Int. Cl. C07K 16/32; C07K 16/28; A61P 35/00

(52) U.S. Cl. CPC C07K 16/32; C07K 16/2809; A61P 35/00; A61K 2039/505; C07K 2317/622; C07K 2317/732; C07K 2317/31

(57) ABSTRACT

Disclosed herein are compositions comprising a recombinant nucleic acid sequence encoding a bispecific immune cell engaging antibody (DICE), a recombinant nucleic acid sequence encoding a bispecific T cell engaging (DBiTE) antibody, a fragment thereof, a variant thereof, or a combination thereof.

### CROSS REFERENCE TO RELATED APPLICATIONS

This application claims priority to U.S. Provisional Application No. 62/798,626, filed Jan. 30, 2019 and to U.S. Provisional Application No. 62/827,265, filed Apr. 1, 2019, each of which is hereby incorporated by reference herein in its entirety.

### TECHNICAL FIELD

The present invention relates to compositions comprising a recombinant nucleic acid sequence for generating one or more synthetic DNA encoded bispecific T cell engagers (BiTE), and functional fragments thereof, in vivo, and methods of preventing and/or treating cancer in a subject by administering said compositions.

### BACKGROUND

Monoclonal antibody therapy has been a game-changer in cancers therapeutics, however, this treatment has several limitations including requirement for repeated administration, more limited stability and cost. A further advance on monoclonal technology is the development of bispecific T cell engagers (BiTE) which combine the specificity of monoclonal antibodies with the cytotoxic potential of T cells. BiTEs have shown promising results in leukemia clinical trials (Viardot et al., 2016, Blood, 127(11):1410-6; Goebeler et al., 2016, J Clin Oncol, 34(10):1104-11), however, this therapy has a limited applicability because it requires continuous intravenous infusion for 4-8 weeks per cycle (Zhu et al., 2016, Clin Pharmacokinet, 55(10):1271-88) and can have limitations for its production. A longer-lived simpler production method for antibody-based products would likely be an important new tool for cancer immunotherapy.

Thus there is need in the art for longer-lived, simpler production, antibody-based products for cancer immunotherapy. The current invention satisfies this need.

### SUMMARY

In one embodiment, the invention relates to a nucleic acid molecule encoding one or more synthetic DNA encoded bispecific immune cell engager, wherein the synthetic DNA encoded bispecific immune cell engager comprises at least one least one antigen binding domain, and at least one immune cell engaging domain.

In one embodiment, the antigen binding domain targets CD19, B-cell maturation antigen (BCMA), CD33, Fibroblast Activation Protein (FAP), follicle stimulating hormone receptor (FSHR), epidermal growth factor receptor (EGFR), prostate-specific membrane antigen (PSMA), CD123 or Her2.

In one embodiment, the immune cell engaging domain targets a T cell, an antigen presenting cell, a natural killer (NK) cell, a neutrophil or a macrophage.

In one embodiment, the immune cell engaging domain targets CD3, the T cell receptor (TCR), CD28, CD16, NKG2D, Ox40, 4-1BB, CD2, CD5, CD40, FcgRs, FceRs, FcaRs or CD95. In one embodiment, the immune cell engaging domain targets CD3.

In one embodiment, the nucleic acid molecule comprises a nucleotide sequence encoding one or more sequences selected from: a) an amino acid sequence having at least about 90% identity over an entire length of the amino acid sequence to an amino acid sequence selected from SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:70, SEQ ID NO:72, SEQ ID NO:74 or SEQ ID NO:76; b) a fragment of an amino acid sequence having at least about 90% identity over at least 65% of the amino acid sequence to an amino acid sequence selected from SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:70, SEQ ID NO:72, SEQ ID NO:74 or SEQ ID NO:76; c) an amino acid sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:70, SEQ ID NO:72, SEQ ID NO:74 or SEQ ID NO:76; and d) a fragment of an amino acid sequence comprising at least 65% of an amino acid sequence selected from SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:70, SEQ ID NO:72, SEQ ID NO:74 or SEQ ID NO:76.

In one embodiment, the nucleic acid molecule comprises: a) a nucleotide sequence having at least about 90% identity over an entire length of the nucleic acid sequence to a nucleotide sequence selected from SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:69, SEQ ID NO:71, SEQ ID NO:73, and SEQ ID NO:75; b) a fragment of a nucleotide sequence having at least about 90% identity over at least 65% of the nucleic acid sequence to a nucleotide sequence selected from SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:69, SEQ ID NO:71, SEQ ID NO:73, and SEQ ID NO:75; c) a nucleotide sequence of SEQ ID NO:1, SEQ ID NO:3, SEQ ID NO:5, SEQ ID NO: 7, SEQ ID NO:9, SEQ ID NO:11, SEQ ID NO: 13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:69, SEQ ID NO:71, SEQ ID NO:73, and SEQ ID NO:75; or d) a fragment of a nucleotide sequence comprising at least 65% of a nucleotide sequence selected from SEQ ID NO: 1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:69, SEQ ID NO:71, SEQ ID NO:73, and SEQ ID NO:75.

In one embodiment, the nucleotide sequence is operably linked to a nucleic acid sequence encoding an IgE leader sequence.

In one embodiment, the nucleic acid molecule comprises an expression vector.

In one embodiment, the invention relates to a composition comprising a nucleic acid molecule encoding one or more synthetic DNA encoded bispecific immune cell engager, wherein the synthetic DNA encoded bispecific immune cell engager comprises at least one least one antigen binding domain, and at least one immune cell engaging domain. In one embodiment, the composition further comprises a pharmaceutically acceptable excipient.

In one embodiment, the invention relates to a method of preventing or treating a disease or disorder in a subject, the method comprising administering to the subject a nucleic acid molecule encoding one or more synthetic DNA encoded bispecific immune cell engager or a composition comprising a nucleic acid molecule encoding one or more synthetic DNA encoded bispecific immune cell engager, wherein the synthetic DNA encoded bispecific immune cell engager comprises at least one least one antigen binding domain, and at least one immune cell engaging domain. In one embodiment, the disease is a benign tumor, cancer or a cancer-associated disease.

In one embodiment, the invention relates to a nucleic acid molecule encoding one or more synthetic antibodies, wherein the nucleic acid molecule comprises a nucleotide sequence encoding an anti-human epidermal growth factor receptor 2 (HER2) synthetic antibody; a nucleotide sequence encoding a fragment of an anti-HER2 synthetic antibody; a nucleotide sequence encoding a ScFv anti-HER2 synthetic antibody; or a nucleotide sequence encoding a fragment of a ScFv anti-HER2 synthetic antibody.

In one embodiment, the nucleotide sequence encodes an amino acid sequence having at least about 90% identity over an entire length of the amino acid sequence to SEQ ID NO:62, SEQ ID NO:64, or SEQ ID NO:66. In one embodiment, the nucleotide sequence encodes a fragment of an amino acid sequence having at least about 90% identity over at least 65% of the amino acid sequence to SEQ ID NO:62, SEQ ID NO:64, or SEQ ID NO:66. In one embodiment, the nucleotide sequence encodes an amino acid sequence of SEQ ID NO:62, SEQ ID NO:64, or. SEQ ID NO:66. In one embodiment, the nucleotide sequence encodes a fragment of an amino acid sequence comprising at least 65% of SEQ ID NO:62, SEQ ID NO:64, or SEQ ID NO:66.

In one embodiment, the nucleic acid molecule comprises a nucleotide sequence having at least about 90% identity over an entire length of SEQ ID NO:61, SEQ ID NO:63, or SEQ ID NO:65. In one embodiment, the nucleic acid molecule comprises a fragment of a nucleotide sequence having at least about 90% identity over at least 65% of the nucleic acid sequence to a nucleotide sequence of SEQ ID NO:61, SEQ ID NO:63, or SEQ ID NO:65. In one embodiment, the nucleic acid molecule comprises a nucleotide sequence selected of SEQ ID NO:61, SEQ ID NO:63, or SEQ ID NO:65. In one embodiment, the nucleic acid molecule comprises a fragment of a nucleotide sequence comprising at least 65% of a nucleotide sequence of SEQ ID NO:61, SEQ ID NO:63, or SEQ ID NO:65.

In one embodiment, the nucleotide sequence is operably linked to a nucleic acid sequence encoding an IgE leader sequence.

In one embodiment, the nucleic acid molecule comprises an expression vector.

In one embodiment, the invention relates to a composition comprising a nucleic acid molecule encoding one or more synthetic antibodies, wherein the nucleic acid molecule comprises a nucleotide sequence encoding an anti-human epidermal growth factor receptor 2 (HER2) synthetic antibody; a nucleotide sequence encoding a fragment of an anti-HER2 synthetic antibody; a nucleotide sequence encoding a ScFv anti-HER2 synthetic antibody; or a nucleotide sequence encoding a fragment of a ScFv anti-HER2 synthetic antibody. In one embodiment, the composition further comprises a pharmaceutically acceptable excipient.

In one embodiment, the invention relates to a method of preventing or treating a disease in a subject, the method comprising administering to the subject a nucleic acid molecule encoding one or more synthetic antibodies, wherein the nucleic acid molecule comprises a nucleotide sequence encoding an anti-human epidermal growth factor receptor 2 (HER2) synthetic antibody; a nucleotide sequence encoding a fragment of an anti-HER2 synthetic antibody; a nucleotide sequence encoding a ScFv anti-HER2 synthetic antibody; or a nucleotide sequence encoding a fragment of a ScFv anti-HER2 synthetic antibody.

In one embodiment, the invention relates to a method of preventing or treating a disease in a subject, the method comprising administering to the subject a nucleic acid molecule encoding one or more synthetic antibodies, wherein the nucleic acid molecule comprises a nucleotide sequence encoding an anti-human epidermal growth factor receptor 2 (HER2) synthetic antibody; a nucleotide sequence encoding a fragment of an anti-HER2 synthetic antibody; a nucleotide sequence encoding a ScFv anti-HER2 synthetic antibody; or a nucleotide sequence encoding a fragment of a ScFv anti-HER2 synthetic antibody. In one embodiment, the disease is a cancer associated with HER2 expression. In one embodiment, the disease is ovarian cancer or breast cancer.

In one embodiment, the invention relates to a method of preventing or treating a disease in a subject, the method comprising administering to the subject a composition comprising a nucleic acid molecule encoding one or more synthetic antibodies, wherein the nucleic acid molecule comprises a nucleotide sequence encoding an anti-human epidermal growth factor receptor 2 (HER2) synthetic antibody; a nucleotide sequence encoding a fragment of an anti-HER2 synthetic antibody; a nucleotide sequence encoding a ScFv anti-HER2 synthetic antibody; or a nucleotide sequence encoding a fragment of a ScFv anti-HER2 synthetic antibody. In one embodiment, the disease is a cancer associated with HER2 expression. In one embodiment, the disease is ovarian cancer or breast cancer.

### BRIEF DESCRIPTION OF THE DRAWINGS

FIG. 1 depicts an exemplary western blot of the supernatant of 293T cells transfected with BCMADBiTE, CD33DBiTE and CD123DBiTE.

FIG. 2 depicts an exemplary western blot of the supernatant of 293T cells transfected with EGFRvIIIDBiTE, FSHRDBiTE, PSMADBiTE and CD19DBiTE.

FIG. 3 depicts a diagram of the experimental design. PBMC from 3 independent donors were cultured in triplicate for 5 hours in the presence of 5 μl of supernatant of CD19DBiTE or a control DBiTE (EGFRvIIIDBiTE). Following incubation, the cells were stained for B cell and T cell markers to determine the potential cytolytic activity against B cells (CD19+ cells) and T cell early activation.

FIG. 4 depicts exemplary experimental results demonstrating that all three donors presented depletion of their B cells (CD19+ cells in the PBMC mix) in the presence of CD19DBiTE but not in the presence of the control DBiTE.

FIG. 5 depicts exemplary experimental results demonstrating that all three donors presented increase in the early activation marker CD69 in their T cells in the presence of CD19DBiTE but not in the presence of the control DBiTE.

FIG. 6, comprising FIG. 6A through FIG. 6F, depicts the design, expression and binding of HER2 DNA encoded monoclonal antibodies (DMAb). FIG. 6A depicts a schematic of DNA construct encoding HER2DMAb. FIG. 6B depicts a western blot of HER2DMAb or FSHR constructs expressed in 293T cells. FIG. 6C depicts a western blot of human IgG from mouse sera electroporated with HER2DMAb or pVax alone 64 days after DNA injection and electroporation (n=5 mice per group). FIG. 6D depicts the expression levels of human IgG quantified by ELISA from sera of nude mice electroporated with HER2DMAb (n=5 mice per group, 2 independent experiments). FIG. 6E depicts a binding ELISA of sera from mice expressing HER2DMAb or pVax after coating the plate with human HER2 protein. FIG. 6F depicts a flow cytometry plot showing binding of HER2DMAb to mouse breast cancer cell lines with and without human HER2 expression.

FIG. 7, comprising FIG. 7A through FIG. 7F, depicts the in vitro expression and anti-tumor activity of HER2 DNA encoded monoclonal antibodies (DMAb).

FIG. 7A depicts the expression levels of HER2DMAb quantified from the supernatant of 293T or RD cells 48 h after DNA transfection (n=3/group). FIG. 7B depicts the in vitro cytotoxicity resulting from culture of human PBMC (0.5 millions) with OVCAR3-luciferase (10,000) cells in the presence of HER2DMAb or pVax sera or Hu4D5 antibody as positive control (triplicates). FIG. 7C depicts the in vitro cytotoxicity resulting from coculture of human PBMC (0.5 millions) with HER2 negative cell line MDA-MD-231 (10,000) cells in the presence of sera from HER2DMAb or pVax injected mice (triplicates). FIG. 7D depicts the percentage of OVCAR3 cells phagocytosed by macrophages in the presence of HER3DMAb, pVax sera or no added sera and representative flow cytometry plots (triplicates). FIG. 7E depicts the in vitro cytotoxicity resulting from coculture of splenocytes from Nu/J mice (0.5 millions) with OVCAR3 (10,000) cells in the presence of sera from HER2DMAb or pVax injected mice (triplicates). FIG. 7F depicts the mouse anti-HER2DMAb IgG at days 0 and 252 in Nu/J sera (triplicates). ANOVA. T-test. ***p<0.001. ns: not significant.

FIG. 8, comprising FIG. 8A through FIG. 8C, shows HER2DMAb binds to HER2 in ovarian cancer. FIG. 8A depicts HER2 expression in ovarian carcinoma cell lines OVCAR3, SKOV3, CAOV3, TOV-21G and RNG1 by flow cytometry using anti-HER2 antibody 24D2. FIG. 8B depicts HER2 expression in sera from mice expressing HER2DMAb. FIG. 8C depicts immunofluorescence imaging of OVCAR3 tumor stained with sera from HER2DMAb expressing mice. Scale bar 10 μm.

FIG. 9, comprising FIG. 9A through FIG. 9F, shows HER2DMAb blocks HER2 signaling, induces ADCC and delays cancer progression in vivo. FIG. 9A depicts a western blot showing total and phosphorylated Akt and R-actin from OVCAR3 cells treated with the HER2-HER3 agonist HRG in the presence of HER2DMAb or control sera. FIG. 9B depicts a histogram showing ADCC assay of HER2DMAb or irrelevant IgG with OVCAR3. FIG. 9C depicts a growth curve of OVCAR3 tumors grafted into nude mice treated with HER2DMAb or empty vector (2 independent experiments of n=5 mice per group). FIG. 9D depicts the levels of HER2DMAb in serum of OVCAR3 bearing mice treated with HER2DMAb or empty vector (representative of 2 independent experiments of n=5 mice per group). FIG. 9E depicts a flow cytometry plot showing expression of HER2 by OVCAR3, Brpkp110 and Brpkp110-hHER2 tumor cells. FIG. 9F depicts a growth curve of Brpkp110-hHER2 tumors grafted into C57Bl/6 mice treated with HER2DMAb or empty pVax plasmid (representative of 2 independent experiments of n=5 mice per group). Two-way ANOVA, t-test, log rank. *p<0.05, ***p<0.001.

FIG. 10, comprising FIG. 10A through FIG. 10J, shows the binding, cytotoxicity, activation and in vivo effectiveness of HER2DBiTE. FIG. 10A depicts HER2DBiTE binding to recombinant HER2 protein measured by binding ELISA (triplicates). FIG. 10B depicts HER2DBiTE binding to recombinant CD3 protein measured by binding ELISA (triplicates). FIG. 10C depicts the number of T cells present in wells 24 hours after coincubation of T cells with OVCAR3 in the presence of HER2DBiTE or pVax sera (triplicates). FIG. 10D depicts the presence of apoptotic (Annexin V+) cells 5 days after activation of T cells with HER2DBIiTE or pVax sera in the presence of OVCAR3 cells. Anti-CD3/anti-CD28 beads were used as positive control (triplicates). FIG. 10E depicts the T cell activation measured as IFNγ in the supernatant of T cells cultured for 24 hours in the presence of HER2DBIiTE or pVax sera and OVCAR3 cells. Anti-CD3/anti-CD28 beads were used as positive control, T cells alone as negative control (triplicates). FIG. 10F depicts T cell activation measured as expression of CD69 in T cells cultured for 72 hours in the presence of HER2DBIiTE or pVax sera and OVCAR3 cells. Anti-CD3/anti-CD28 beads were used as positive control, T cells alone as negative control (trilpicates). FIG. 10G depicts T cell activation measured as expression of PD-1 in T cells cultured for 72 hours in the presence of HER2DBIiTE or pVax sera and OVCAR3 cells. Anti-CD3/anti-CD28 beads were used as positive control, T cells alone as negative control (triplicates). FIG. 10H depicts in vitro cytotoxicity resulting from coculture of T cells with OVCAR3 cells at different ratios in the presence of sera from HER2DBiTE or pVax mice (2 independent experiments in triplicate). FIG. 10I depicts mouse anti-HER2DBiTE IgG at days 0 and 64 in Nu/J sera (trilpicates). FIG. 10J depicts an average growth curve of OVCAR3 tumors grafted into NSG mice treated with HER2DBiTE or empty vector without PBMC and HER2DBiTE with PBMC and image of tumors (n=5 mice per group; X denotes no tumor (full rejection)). T-test, ANOVA, Two-way ANOVA. *p<0.05, **p<0.01 ***p<0.001. ns: not significant.

FIG. 11, comprising FIG. 11A through FIG. 11F, shows the generation, expression and anti-tumor activity of HER2DBiTE. FIG. 11A depicts a schematic of DNA construct encoding HER2DMAb and cartoon of BiTE engaging HER2 and the TCR. FIG. 11B depicts a western blot of human IgG from 1 μl of mouse sera electroporated with HER2DBiTE or pVax empty vector 21 and 28 days after DNA injection and electroporation (representative of n=5 mice per group). FIG. 11C depicts in vitro cytotoxicity resulting from coculture of T cells with OVCAR3 cells at different ratios in the presence of sera from HER2DBiTE or pVax mice (2 independent experiments in triplicate). FIG. 11D depicts in vitro cytotoxicity of sera from mice treated with HER2DBiTE before and at different time points after injection and electroporation of 100 μg at an effector:target ratio of 5:1 using OVCAR3 as target (triplicates). FIG. 11E depicts an average growth curve of OVCAR3 tumors grafted into NSG mice treated with HER2DBiTE or empty vector (n=10 mice per group). FIG. 11F depicts individual growth curves of OVCAR3 tumors grafted into NSG mice treated with HER2DBiTE or empty vector (n=10 mice per group). Two-way ANOVA. ***p<0.001.

### DETAILED DESCRIPTION

The present invention relates to compositions comprising a recombinant nucleic acid sequence encoding a bispecific immune cell engaging antibody (DICE), a recombinant nucleic acid sequence encoding a bispecific T cell engaging (DBiTE) antibody, a fragment thereof, a variant thereof, or a combination thereof. The composition can be administered to a subject in need thereof to facilitate in vivo expression and formation of a DICE or DBiTE.

In one embodiment, the DICE or DBiTE comprises at least one antigen binding domain, and at least one immune cell engaging domain. In one embodiment, the immune cell engaging domain is specific for an antigen expressed on the surface of an immune cell. Immune cells include, but are not limited to, T cells, antigen presenting cells, NK cells, neutrophils and macrophages.

In various embodiments, the immune cell engaging domain comprises a nucleotide sequence encoding an antibody, a fragment thereof, or a variant thereof specific for binding to a immune cell specific receptor molecule. In one embodiment, the immune cell specific receptor molecule is a T cell surface antigen. In one embodiment, the T cell specific receptor molecule is one of CD3, TCR, CD28, CD16, NKG2D, Ox40, 4-1BB, CD2, CD5, CD40, FcgRs, FceRs, FcaRs and CD95.

In various embodiments, the antigen binding domain comprises a nucleotide sequence encoding an antibody, a fragment thereof, or a variant thereof specific for binding to an antigen. In one embodiment, the antibody or fragment thereof is a DNA encoded monoclonal antibody (DMAb) or a fragment or variant thereof.

In one embodiment, the antigen binding domain of the DICE or DBiTE is specific for binding a target antigen, and recruiting a T cell to the target antigen. In one embodiment, the target antigen is a tumor antigen. In one embodiment, the antigen is CD19, B-cell maturation antigen (BCMA), CD33, Fibroblast Activation Protein (FAP), follicle stimulating hormone receptor (FSHR), epidermal growth factor receptor (EGFR), prostate-specific membrane antigen (PSMA), CD123 and human epidermal growth factor receptor 2 (Her2). Therefore, in one embodiment, the invention provides compositions comprising one or more DICE or DBiTE and methods for use in treating or preventing cancer or a disease or disorder associated with cancer in a subject.

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

“Antibody” may mean an antibody of classes IgG, IgM, IgA, IgD or IgE, or fragments, fragments or derivatives thereof, including Fab, F(ab′)2, Fd, and single chain antibodies, and derivatives thereof. The antibody may be an antibody isolated from the serum sample of mammal, a polyclonal antibody, affinity purified antibody, or mixtures thereof which exhibits sufficient binding specificity to a desired epitope or a sequence derived therefrom.

“Antibody fragment” or “fragment of an antibody” as used interchangeably herein refers to a portion of an intact antibody comprising the antigen-binding site or variable region. The portion does not include the constant heavy chain domains (i.e. CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)2 fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

“Antigen” refers to proteins that have the ability to generate an immune response in a host. An antigen may be recognized and bound by an antibody. An antigen may originate from within the body or from the external environment.

“Coding sequence” or “encoding nucleic acid” as used herein may mean refers to the nucleic acid (RNA or DNA molecule) that comprise a nucleotide sequence which encodes an antibody as set forth herein. The coding sequence may further include initiation and termination signals operably linked to regulatory elements including a promoter and polyadenylation signal capable of directing expression in the cells of an individual or mammal to whom the nucleic acid is administered. The coding sequence may further include sequences that encode signal peptides.

“Complement” or “complementary” as used herein may mean a nucleic acid may mean Watson-Crick (e.g., A-T/U and C-G) or Hoogsteen base pairing between nucleotides or nucleotide analogs of nucleic acid molecules.

“Constant current” as used herein to define a current that is received or experienced by a tissue, or cells defining said tissue, over the duration of an electrical pulse delivered to same tissue. The electrical pulse is delivered from the electroporation devices described herein. This current remains at a constant amperage in said tissue over the life of an electrical pulse because the electroporation device provided herein has a feedback element, preferably having instantaneous feedback. The feedback element can measure the resistance of the tissue (or cells) throughout the duration of the pulse and cause the electroporation device to alter its electrical energy output (e.g., increase voltage) so current in same tissue remains constant throughout the electrical pulse (on the order of microseconds), and from pulse to pulse. In some embodiments, the feedback element comprises a controller.

“Current feedback” or “feedback” as used herein may be used interchangeably and may mean the active response of the provided electroporation devices, which comprises measuring the current in tissue between electrodes and altering the energy output delivered by the EP device accordingly in order to maintain the current at a constant level. This constant level is preset by a user prior to initiation of a pulse sequence or electrical treatment. The feedback may be accomplished by the electroporation component, e.g., controller, of the electroporation device, as the electrical circuit therein is able to continuously monitor the current in tissue between electrodes and compare that monitored current (or current within tissue) to a preset current and continuously make energy-output adjustments to maintain the monitored current at preset levels. The feedback loop may be instantaneous as it is an analog closed-loop feedback.

“Decentralized current” as used herein may mean the pattern of electrical currents delivered from the various needle electrode arrays of the electroporation devices described herein, wherein the patterns minimize, or preferably eliminate, the occurrence of electroporation related heat stress on any area of tissue being electroporated.

“Electroporation,” “electro-permeabilization,” or “electro-kinetic enhancement” (“EP”) as used interchangeably herein may refer to the use of a transmembrane electric field pulse to induce microscopic pathways (pores) in a bio-membrane; their presence allows biomolecules such as plasmids, oligonucleotides, siRNA, drugs, ions, and water to pass from one side of the cellular membrane to the other.

“Endogenous antibody” as used herein may refer to an antibody that is generated in a subject that is administered an effective dose of an antigen for induction of a humoral immune response.

“Feedback mechanism” as used herein may refer to a process performed by either software or hardware (or firmware), which process receives and compares the impedance of the desired tissue (before, during, and/or after the delivery of pulse of energy) with a present value, preferably current, and adjusts the pulse of energy delivered to achieve the preset value. A feedback mechanism may be performed by an analog closed loop circuit.

“Fragment” may mean a polypeptide fragment of an antibody that is function, i.e., can bind to desired target and have the same intended effect as a full length antibody. A fragment of an antibody may be 100% identical to the full length except missing at least one amino acid from the N and/or C terminal, in each case with or without signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length antibody, excluding any heterologous signal peptide added. The fragment may comprise a fragment of a polypeptide that is 95% or more, 96% or more, 97% or more, 98% or more or 99% or more identical to the antibody and additionally comprise an N terminal methionine or heterologous signal peptide which is not included when calculating percent identity. Fragments may further comprise an N terminal methionine and/or a signal peptide such as an immunoglobulin signal peptide, for example an IgE or IgG signal peptide. The N terminal methionine and/or signal peptide may be linked to a fragment of an antibody.

A fragment of a nucleic acid sequence that encodes an antibody may be 100% identical to the full length except missing at least one nucleotide from the 5′ and/or 3′ end, in each case with or without sequences encoding signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length coding sequence, excluding any heterologous signal peptide added. The fragment may comprise a fragment that encode a polypeptide that is 95% or more, 96% or more, 97% or more, 98% or more or 99% or more identical to the antibody and additionally optionally comprise sequence encoding an N terminal methionine or heterologous signal peptide which is not included when calculating percent identity. Fragments may further comprise coding sequences for an N terminal methionine and/or a signal peptide such as an immunoglobulin signal peptide, for example an IgE or IgG signal peptide. The coding sequence encoding the N terminal methionine and/or signal peptide may be linked to a fragment of coding sequence.

“Genetic construct” as used herein refers to the DNA or RNA molecules that comprise a nucleotide sequence which encodes a protein, such as an antibody. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

“Identical” or “identity” as used herein in the context of two or more nucleic acids or polypeptide sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Impedance” as used herein may be used when discussing the feedback mechanism and can be converted to a current value according to Ohm's law, thus enabling comparisons with the preset current.

“Immune response” as used herein may mean the activation of a host's immune system, e.g., that of a mammal, in response to the introduction of one or more nucleic acids and/or peptides. The immune response can be in the form of a cellular or humoral response, or both.

“Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

“Operably linked” as used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

A “peptide,” “protein,” or “polypeptide” as used herein can mean a linked sequence of amino acids and can be natural, synthetic, or a modification or combination of natural and synthetic.

“Promoter” as used herein may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV 40 late promoter and the CMV IE promoter.

“Signal peptide” and “leader sequence” are used interchangeably herein and refer to an amino acid sequence that can be linked at the amino terminus of a protein set forth herein. Signal peptides/leader sequences typically direct localization of a protein. Signal peptides/leader sequences used herein preferably facilitate secretion of the protein from the cell in which it is produced. Signal peptides/leader sequences are often cleaved from the remainder of the protein, often referred to as the mature protein, upon secretion from the cell. Signal peptides/leader sequences are linked at the N terminus of the protein.

“Stringent hybridization conditions” as used herein may mean conditions under which a first nucleic acid sequence (e.g., probe) will hybridize to a second nucleic acid sequence (e.g., target), such as in a complex mixture of nucleic acids. Stringent conditions are sequence dependent and will be different in different circumstances. Stringent conditions may be selected to be about 5-10° C. lower than the thermal melting point (Tₘ) for the specific sequence at a defined ionic strength pH. The Tₘ may be the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc) and a human). In some embodiments, the subject may be a human or a non-human. The subject or patient may be undergoing other forms of treatment.

“Substantially complementary” as used herein may mean that a first sequence is at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or that the two sequences hybridize under stringent hybridization conditions.

“Substantially identical” as used herein may mean that a first and second sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more nucleotides or amino acids, or with respect to nucleic acids, if the first sequence is substantially complementary to the complement of the second sequence.

“Synthetic antibody” as used herein refers to an antibody that is encoded by the recombinant nucleic acid sequence described herein and is generated in a subject.

“Treatment” or “treating,” as used herein can mean protecting of a subject from a disease through means of preventing, suppressing, repressing, or completely eliminating the disease. Preventing the disease involves administering an antibody of the present invention to a subject prior to onset of the disease. Suppressing the disease involves administering a antibody of the present invention to a subject after induction of the disease but before its clinical appearance. Repressing the disease involves administering an antibody of the present invention to a subject after clinical appearance of the disease.

“Variant” used herein with respect to a nucleic acid may mean (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of 2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant may be a nucleic acid sequence that is substantially identical over the full length of the full gene sequence or a fragment thereof. The nucleic acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant may be an amino acid sequence that is substantially identical over the full length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

“Vector” as used herein may mean a nucleic acid sequence containing an origin of replication. A vector may be a plasmid, bacteriophage, bacterial artificial chromosome or yeast artificial chromosome. A vector may be a DNA or RNA vector. A vector may be either a self-replicating extrachromosomal vector or a vector which integrates into a host genome.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Compositions

In one embodiment, the present invention relates to compositions comprising a recombinant nucleic acid sequence encoding a DICE or DBiTE, a fragment thereof, a variant thereof, or a combination thereof. The compositions, when administered to a subject in need thereof, can result in the generation of a synthetic DNA encoded bispecific immune cell engager in the subject.

In one embodiment, the DICE or DBiTE comprises at least one antigen binding domain, and at least one immune cell engaging domain. In one embodiment, the immune cell engaging domain is specific for an antigen expressed on the surface of an immune cell. Immune cells include, but are not limited to, T cells, antigen presenting cells, NK cells, neutrophils and macrophages.

In various embodiments, the immune cell engaging domain comprises a nucleotide sequence encoding an antibody, a fragment thereof, or a variant thereof specific for binding to a immune cell specific receptor molecule. In one embodiment, the immune cell specific receptor molecule is a T cell surface antigen. In one embodiment, the T cell specific receptor molecule is one of CD3, TCR, CD28, CD16, NKG2D, Ox40, 4-1BB, CD2, CD5, CD40, FcgRs, FceRs, FcaRs and CD95.

In various embodiments, the antigen binding domain comprises an antibody, a fragment thereof, or a variant thereof specific for binding to an antigen. In one embodiment, the antigen is a tumor antigen. In one embodiment, the antigen is CD19, B-cell maturation antigen (BCMA), CD33, Fibroblast Activation Protein (FAP), follicle stimulating hormone receptor (FSHR), epidermal growth factor receptor (EGFR), prostate-specific membrane antigen (PSMA), CD123 or human epidermal growth factor receptor 2 (Her2).

In one embodiment, a nucleotide sequence encoding a CD19DBiTE encodes the amino acid sequence of SEQ ID NO:2, SEQ ID NO:4 or SEQ ID NO:6 or a fragment or variant thereof. In one embodiment, a nucleotide sequence encoding a CD19DBiTE comprises a nucleotide sequence of SEQ ID NO:1, SEQ ID NO:3 or SEQ ID NO:5 or a fragment or variant thereof.

In one embodiment, a nucleotide sequence encoding a BCMADBiTE encodes the amino acid sequence of SEQ ID NO:8, SEQ ID NO:10 or SEQ ID NO:12 or a fragment or variant thereof. In one embodiment, a nucleotide sequence encoding a BCMADBiTE comprises a nucleotide sequence of SEQ ID NO:7, SEQ ID NO:9 or SEQ ID NO:11 or a fragment or variant thereof.

In one embodiment, a nucleotide sequence encoding a CD33DBiTE encodes the amino acid sequence of SEQ ID NO:14, SEQ ID NO: 16 or SEQ ID NO:18 or a fragment or variant thereof. In one embodiment, a nucleotide sequence encoding a CD33DBiTE comprises a nucleotide sequence of SEQ ID NO:13, SEQ ID NO:15 or SEQ ID NO:17 or a fragment or variant thereof.

In one embodiment, a nucleotide sequence encoding a FAPBiTE encodes the amino acid sequence of SEQ ID NO:20, SEQ ID NO:22, or SEQ ID NO:24 or a fragment or variant thereof. In one embodiment, a nucleotide sequence encoding a FAPDBiTE comprises a nucleotide sequence of SEQ ID NO:19, SEQ ID NO:21 or SEQ ID NO:23 or a fragment or variant thereof.

In one embodiment, a nucleotide sequence encoding a FSHRDBiTE encodes the amino acid sequence of SEQ ID NO:26, SEQ ID NO:28, or SEQ ID NO:30 or a fragment or variant thereof. In one embodiment, a nucleotide sequence encoding a FSHRDBiTE comprises a nucleotide sequence of SEQ ID NO:25, SEQ ID NO:27 or SEQ ID NO:29 or a fragment or variant thereof.

In one embodiment, a nucleotide sequence encoding a EGFRDBiTE encodes the amino acid sequence of SEQ ID NO:32, SEQ ID NO:34, or SEQ ID NO:36 or a fragment or variant thereof. In one embodiment, a nucleotide sequence encoding a EGFRDBiTE comprises a nucleotide sequence of SEQ ID NO:31, SEQ ID NO:33 or SEQ ID NO:35 or a fragment or variant thereof.

In one embodiment, a nucleotide sequence encoding a PSMADBiTE encodes the amino acid sequence of SEQ ID NO:38, SEQ ID NO:40, or SEQ ID NO:42 or a fragment or variant thereof. In one embodiment, a nucleotide sequence encoding a PSMADBiTE comprises a nucleotide sequence of SEQ ID NO:37, SEQ ID NO:41, or SEQ ID NO:43 or a fragment or variant thereof.

In one embodiment, a nucleotide sequence encoding a CD123DBiTE encodes the amino acid sequence of SEQ ID NO:44, SEQ ID NO:46 or SEQ ID NO:48 or a fragment or variant thereof. In one embodiment, a nucleotide sequence encoding a CD123DBiTE comprises a nucleotide sequence of SEQ ID NO:43, SEQ ID NO:45 or SEQ ID NO:47 or a fragment or variant thereof.

In one embodiment, a nucleotide sequence encoding a HER2DBiTE encodes the amino acid sequence of SEQ ID NO:50, SEQ ID NO:52, SEQ ID NO:55, SEQ ID NO:56, SEQ ID NO:58 or SEQ ID NO:60 or a fragment or variant thereof. In one embodiment, a nucleotide sequence encoding a HER2DBiTE comprises a nucleotide sequence of SEQ ID NO:49, SEQ ID NO:51, SEQ ID NO:53, SEQ ID NO:55, SEQ ID NO:57, SEQ ID NO:59 or SEQ ID NO:67 or a fragment or variant thereof.

In one embodiment, a nucleotide sequence encoding a EGFRvIII2DICE encodes the amino acid sequence of SEQ ID NO:70 or SEQ ID NO:72 or a fragment or variant thereof. In one embodiment, a nucleotide sequence encoding a EGFRvIII2DICE comprises a nucleotide sequence of SEQ ID NO:69 or SEQ ID NO:71 or a fragment or variant thereof.

In one embodiment, a nucleotide sequence encoding a HER2DICE encodes the amino acid sequence of SEQ ID NO:74 or SEQ ID NO:76 or a fragment or variant thereof. In one embodiment, a nucleotide sequence encoding a HER2DICE comprises a nucleotide sequence of SEQ ID NO:73 or SEQ ID NO:75 or a fragment or variant thereof.

In one embodiment, the composition comprises a nucleotide sequence encoding an an-Her2 antibody (HER2DMAb). In one embodiment, the nucleotide sequence encoding the HER2DMAb comprises a nucleotide sequence encoding SEQ ID NO:62, SEQ ID NO: 64 or a fragment or variant thereof. In one embodiment, the nucleotide sequence encoding a HER2DMAb comprises SEQ ID NO:61, SEQ ID NO:63 or a fragment or variant thereof.

In one embodiment the composition comprises an scFv anti-Her2 antibody. In one embodiment, the nucleotide sequence encoding the scFv anti-Her2 antibody comprises a nucleotide sequence encoding SEQ ID NO:66 or a fragment or variant thereof. In one embodiment, the nucleotide sequence encoding scFv anti-Her2 antibody comprises SEQ ID NO:65 or a fragment or variant thereof.

TABLE 1
Synthetic Antibody Sequences

| SEQ | | | |
|---|---|---|---|
| ID | Sequence | | Antibody |
| NO: | Type | Description | Type |
| 1 | Nucleotide | CD19xCd3Blin | DBiTE |
| 2 | Amino Acid | CD19xCd3Blin | DBiTE |
| 3 | Nucleotide | CD19xCd3Blin with IgE | DBiTE |
| 4 | Amino Acid | CD19xCd3Blin with IgE | DBiTE |
| 5 | Nucleotide | CD19xCd3Blin with IgE and His tag | DBiTE |
| 6 | Amino Acid | CD19xCd3Blin with IgE and His tag | DBiTE |
| 7 | Nucleotide | BCMAxCD3 | DBiTE |
| 8 | Amino Acid | BCMAxCD3 | DBiTE |
| 9 | Nucleotide | BCMAxCD3 with IgE | DBiTE |
| 10 | Amino Acid | BCMAxCD3 with IgE | DBiTE |
| 11 | Nucleotide | BCMAxCD3 with IgE and His tag | DBiTE |
| 12 | Amino Acid | BCMAxCD3 with IgE and His tag | DBiTE |
| 13 | Nucleotide | CD33xCD3 | DBiTE |
| 14 | Amino Acid | CD33xCD3 | DBiTE |
| 15 | Nucleotide | CD33xCD3 with IgE | DBiTE |
| 16 | Amino Acid | CD33xCD3 with IgE | DBiTE |
| 17 | Nucleotide | CD33xCD3 with IgE and His tag | DBiTE |
| 18 | Amino Acid | CD33xCD3 with IgE and His tag | DBiTE |
| 19 | Nucleotide | FAP4G8xCD3 | DBiTE |
| 20 | Amino Acid | FAP4G8xCD3 | DBiTE |
| 21 | Nucleotide | FAP4G8xCD3 with IgE | DBiTE |
| 22 | Amino Acid | FAP4G8xCD3 with IgE | DBiTE |
| 23 | Nucleotide | FAP4G8xCD3 with IgE and His tag | DBiTE |
| 24 | Amino Acid | FAP4G8xCD3 with IgE and His tag | DBiTE |
| 25 | Nucleotide | FSH33-53xCD3 | DBiTE |
| 26 | Amino Acid | FSH33-53xCD3 | DBiTE |
| 27 | Nucleotide | FSH33-53xCD3 with IgE | DBiTE |
| 28 | Amino Acid | FSH33-53xCD3 with IgE | DBiTE |
| 29 | Nucleotide | FSH33-53xCD3 with IgE and His tag | DBiTE |
| 30 | Amino Acid | FSH33-53xCD3 with IgE and His tag | DBiTE |
| 31 | Nucleotide | EGFRvIIIXCD3 | DBiTE |
| 32 | Amino Acid | EGFRvIIIXCD3 | DBiTE |
| 33 | Nucleotide | EGFRvIIIXCD3 with IgE | DBiTE |
| 34 | Amino Acid | EGFRvIIIXCD3 with IgE | DBiTE |
| 35 | Nucleotide | EGFRvIIIXCD3 with IgE and His tag | DBiTE |
| 36 | Amino Acid | EGFRvIIIXCD3 with IgE and His tag | DBiTE |
| 37 | Nucleotide | PSMAXCD3 | DBiTE |
| 38 | Amino Acid | PSMAXCD3 | DBiTE |
| 39 | Nucleotide | PSMAXCD3 with IgE | DBiTE |
| 40 | Amino Acid | PSMAXCD3 with IgE | DBiTE |
| 41 | Nucleotide | PSMAXCD3 with IgE and His tag | DBiTE |
| 42 | Amino Acid | PSMAXCD3 with IgE and His tag | DBiTE |
| 43 | Nucleotide | CD3xCD123 | DBiTE |
| 44 | Amino Acid | CD3xCD123 | DBiTE |
| 45 | Nucleotide | CD3xCD123 with IgE | DBiTE |
| 46 | Amino Acid | CD3xCD123 with IgE | DBiTE |
| 47 | Nucleotide | CD3xCD123 with IgE and His tag | DBiTE |
| 48 | Amino Acid | CD3xCD123 with IgE and His tag | DBiTE |
| 49 | Nucleotide | HER2DBiTE | DBiTE |
| 50 | Amino Acid | HER2DBiTE | DBiTE |
| 51 | Nucleotide | HER2DBiTE with IgE | DBiTE |
| 52 | Amino Acid | HER2DBiTE with IgE | DBiTE |
| 53 | Nucleotide | Human CD3xHer2 (HER2DBiTE) with | DBiTE |
| | | His tag | |
| 54 | Amino Acid | Human CD3xHer2 (HER2DBiTE) with | DBiTE |
| | | His tag | |
| 55 | Nucleotide | Human CD3xHer2 (HER2DBiTE) with | DBiTE |
| | | IgE leader | |
| 56 | Amino Acid | Human CD3xHer2 (HER2DBiTE) with | DBiTE |
| | | IgE leader | |
| 57 | Nucleotide | HER2DBiTE-L | DBiTE |
| 58 | Amino Acid | HER2DBiTE-L | DBiTE |
| 59 | Nucleotide | HER2DBiTE-L with His tag | DBiTE |
| 60 | Amino Acid | HER2DBiTE-L with His tag | DBiTE |
| 61 | Nucleotide | HER2DMAb | DMAb |
| 62 | Amino Acid | HER2DMAb | DMAb |
| 63 | Nucleotide | HER2DMAb | DMAb |
| 64 | Amino Acid | HER2DMAb | DMAb |
| 65 | Nucleotide | HER2DMAb (scFv) | scFv |
| | | | fragment |
| 66 | Amino Acid | HER2DMAb (scFv) | scFv |
| | | | fragment |
| 67 | Nucleotide | pGX93237 full plasmid sequence | DBiTE |
| 69 | Nucleotide | EGFRvIII-DICE | DICE |
| 70 | Amino Acid | EGFRvIII-DICE | DICE |
| 71 | Nucleotide | EGFRvIII-DICE with IgE leader | DICE |
| 72 | Amino Acid | EGFRvIII-DICE with IgE leader | DICE |
| 73 | Nucleotide | Her2DICE | DICE |
| 74 | Amino Acid | Her2DICE | DICE |
| 75 | Nucleotide | Her2DICE with IgE leader | DICE |
| 76 | Amino Acid | Her2DICE with IgE leader | DICE |

In certain embodiments, the composition can treat, prevent, and or/protect against a disease or disorder associated with the antigen to which the synthetic antibody (e.g., DMAb, ScFv antibody fragment, DICE or DBiTE) of the invention binds. In one embodiment, the composition of the invention can treat, prevent and/or protect against any disease, disorder, or condition associated with expression of the targeted antigen. In certain embodiments, the composition can treat, prevent, and or/protect against cancer.

The synthetic antibody (e.g., DMAb, ScFv antibody fragment, DICE or DBiTE) can treat, prevent, and/or protect against disease in the subject administered the composition. The synthetic antibody (e.g., DMAb, ScFv antibody fragment, DICE or DBiTE) can promote survival of the disease in the subject administered the composition. The synthetic antibody (e.g., DMAb, ScFv antibody fragment, DICE or DBiTE) can provide at least about 50%, 55%, 60%, 65%, 70%, 75%, 80%, 85%, 90%, 95%, or 100% survival of the disease in the subject administered the composition. In other embodiments, the synthetic antibody (e.g., DMAb, ScFv antibody fragment, DICE or DBiTE) can provide at least about 65%, 66%, 67%, 68%, 69%, 70%, 71%, 72%, 73%, 74%, 75%, 76%, 77%, 78%, 79%, or 80% survival of the disease in the subject administered the composition.

The composition can result in the generation of the synthetic antibody (e.g., DMAb, ScFv antibody fragment, DICE or DBiTE) in the subject within at least about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 20 hours, 25 hours, 30 hours, 35 hours, 40 hours, 45 hours, 50 hours, or 60 hours of administration of the composition to the subject. The composition can result in generation of the synthetic antibody (e.g., DMAb, ScFv antibody fragment, DICE or DBiTE) in the subject within at least about 1 day, 2 days, 3 days, 4 days, 5 days, 6 days, 7 days, 8 days, 9 days, or 10 days of administration of the composition to the subject. The composition can result in generation of the synthetic antibody (e.g., DMAb, ScFv antibody fragment, DICE or DBiTE) in the subject within about 1 hour to about 6 days, about 1 hour to about 5 days, about 1 hour to about 4 days, about 1 hour to about 3 days, about 1 hour to about 2 days, about 1 hour to about 1 day, about 1 hour to about 72 hours, about 1 hour to about 60 hours, about 1 hour to about 48 hours, about 1 hour to about 36 hours, about 1 hour to about 24 hours, about 1 hour to about 12 hours, or about 1 hour to about 6 hours of administration of the composition to the subject.

The composition, when administered to the subject in need thereof, can result in the generation of the synthetic antibody (e.g., DMAb, ScFv antibody fragment, DICE or DBiTE) in the subject more quickly than the generation of an endogenous antibody in a subject who is administered an antigen to induce a humoral immune response. The composition can result in the generation of the synthetic antibody (e.g., DMAb, ScFv antibody fragment, DICE or DBiTE) at least about 1 day, 2 days, 3 days, 4 days, 5 days, 6 days, 7 days, 8 days, 9 days, or 10 days before the generation of the endogenous antibody in the subject who was administered an antigen to induce a humoral immune response.

The composition of the present invention can have features required of effective compositions such as being safe so that the composition does not cause illness or death; being protective against illness; and providing ease of administration, few side effects, biological stability and low cost per dose.

Recombinant Nucleic Acid Sequence

As described above, the composition can comprise a recombinant nucleic acid sequence. The recombinant nucleic acid sequence can encode the synthetic antibody (e.g., DMAb, ScFv antibody fragment, DICE or DBiTE), a fragment thereof, a variant thereof, or a combination thereof. The antibody is described in more detail below.

The recombinant nucleic acid sequence can be a heterologous nucleic acid sequence. The recombinant nucleic acid sequence can include at least one heterologous nucleic acid sequence or one or more heterologous nucleic acid sequences.

The recombinant nucleic acid sequence can be an optimized nucleic acid sequence. Such optimization can increase or alter the immunogenicity of the antibody. Optimization can also improve transcription and/or translation. Optimization can include one or more of the following: low GC content leader sequence to increase transcription; mRNA stability and codon optimization; addition of a kozak sequence (e.g., GCC ACC) for increased translation; addition of an immunoglobulin (Ig) leader sequence encoding a signal peptide; and eliminating to the extent possible cis-acting sequence motifs (i.e., internal TATA boxes).

The recombinant nucleic acid sequence can include one or more recombinant nucleic acid sequence constructs. The recombinant nucleic acid sequence construct can include one or more components, which are described in more detail below.

The recombinant nucleic acid sequence construct can include a heterologous nucleic acid sequence that encodes a heavy chain polypeptide, a fragment thereof, a variant thereof, or a combination thereof. The recombinant nucleic acid sequence construct can include a heterologous nucleic acid sequence that encodes a light chain polypeptide, a fragment thereof, a variant thereof, or a combination thereof. The recombinant nucleic acid sequence construct can also include a heterologous nucleic acid sequence that encodes a protease or peptidase cleavage site. The recombinant nucleic acid sequence construct can also include a heterologous nucleic acid sequence that encodes an internal ribosome entry site (IRES). An IRES may be either a viral IRES or an eukaryotic IRES. The recombinant nucleic acid sequence construct can include one or more leader sequences, in which each leader sequence encodes a signal peptide. The recombinant nucleic acid sequence construct can include one or more promoters, one or more introns, one or more transcription termination regions, one or more initiation codons, one or more termination or stop codons, and/or one or more polyadenylation signals. The recombinant nucleic acid sequence construct can also include one or more linker or tag sequences. The tag sequence can encode a hemagglutinin (HA) tag.

Heavy Chain Polypeptide

The recombinant nucleic acid sequence construct can include a heterologous nucleic acid encoding a heavy chain polypeptide, a fragment thereof, a variant thereof, or a combination thereof. The heavy chain polypeptide can include a variable heavy chain (VH) region and/or at least one constant heavy chain (CH) region. The at least one constant heavy chain region can include a constant heavy chain region 1 (CH1), a constant heavy chain region 2 (CH2), and a constant heavy chain region 3 (CH3), and/or a hinge region.

In some embodiments, the heavy chain polypeptide can include a VH region and a CH1 region. In other embodiments, the heavy chain polypeptide can include a VH region, a CH1 region, a hinge region, a CH2 region, and a CH3 region.

The heavy chain polypeptide can include a complementarity determining region (“CDR”) set. The CDR set can contain three hypervariable regions of the VH region. Proceeding from N-terminus of the heavy chain polypeptide, these CDRs are denoted “CDR1,” “CDR2,” and “CDR3,” respectively. CDR1, CDR2, and CDR3 of the heavy chain polypeptide can contribute to binding or recognition of the antigen.

Light Chain Polypeptide

The recombinant nucleic acid sequence construct can include a heterologous nucleic acid sequence encoding a light chain polypeptide, a fragment thereof, a variant thereof, or a combination thereof. The light chain polypeptide can include a variable light chain (VL) region and/or a constant light chain (CL) region.

The light chain polypeptide can include a complementarity determining region (“CDR”) set. The CDR set can contain three hypervariable regions of the VL region. Proceeding from N-terminus of the light chain polypeptide, these CDRs are denoted “CDR1,” “CDR2,” and “CDR3,” respectively. CDR1, CDR2, and CDR3 of the light chain polypeptide can contribute to binding or recognition of the antigen.

Protease Cleavage Site

The recombinant nucleic acid sequence construct can include the heterologous nucleic acid sequence encoding the protease cleavage site. The protease cleavage site can be recognized by a protease or peptidase. The protease can be an endopeptidase or endoprotease, for example, but not limited to, furin, elastase, HtrA, calpain, trypsin, chymotrypsin, trypsin, and pepsin. The protease can be furin. In other embodiments, the protease can be a serine protease, a threonine protease, cysteine protease, aspartate protease, metalloprotease, glutamic acid protease, or any protease that cleaves an internal peptide bond (i.e., does not cleave the N-terminal or C-terminal peptide bond).

The protease cleavage site can include one or more amino acid sequences that promote or increase the efficiency of cleavage. The one or more amino acid sequences can promote or increase the efficiency of forming or generating discrete polypeptides. The one or more amino acids sequences can include a 2A peptide sequence.

Linker Sequence

The recombinant nucleic acid sequence construct can include one or more linker sequences. The linker sequence can spatially separate or link the one or more components described herein. In other embodiments, the linker sequence can encode an amino acid sequence that spatially separates or links two or more polypeptides. In one embodiment, the linker sequence is a G4S linker sequence, having an amino acid sequence of GGGGSGGGGSGGGGS (SEQ ID NO:68).

Promoter

The recombinant nucleic acid sequence construct can include one or more promoters. The one or more promoters may be any promoter that is capable of driving gene expression and regulating gene expression. Such a promoter is a cis-acting sequence element required for transcription via a DNA dependent RNA polymerase. Selection of the promoter used to direct gene expression depends on the particular application. The promoter may be positioned about the same distance from the transcription start in the recombinant nucleic acid sequence construct as it is from the transcription start site in its natural setting. However, variation in this distance may be accommodated without loss of promoter function.

The promoter may be operably linked to the heterologous nucleic acid sequence encoding the heavy chain polypeptide and/or light chain polypeptide. The promoter may be a promoter shown effective for expression in eukaryotic cells. The promoter operably linked to the coding sequence may be a CMV promoter, a promoter from simian virus 40 (SV40), such as SV40 early promoter and SV40 later promoter, a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human actin, human myosin, human hemoglobin, human muscle creatine, human polyhedrin, or human metalothionein.

The promoter can be a constitutive promoter or an inducible promoter, which initiates transcription only when the host cell is exposed to some particular external stimulus. In the case of a multicellular organism, the promoter can also be specific to a particular tissue or organ or stage of development. The promoter may also be a tissue specific promoter, such as a muscle or skin specific promoter, natural or synthetic. Examples of such promoters are described in US patent application publication no. US20040175727, the contents of which are incorporated herein in its entirety.

The promoter can be associated with an enhancer. The enhancer can be located upstream of the coding sequence. The enhancer may be human actin, human myosin, human hemoglobin, human muscle creatine or a viral enhancer such as one from CMV, FMDV, RSV or EBV. Polynucleotide function enhances are described in U.S. Pat. Nos. 5,593,972, 5,962,428, and WO94/016737, the contents of each are fully incorporated by reference.

Transcription Termination Region

The recombinant nucleic acid sequence construct can include one or more transcription termination regions. The transcription termination region can be downstream of the coding sequence to provide for efficient termination. The transcription termination region can be obtained from the same gene as the promoter described above or can be obtained from one or more different genes.

Initiation Codon

The recombinant nucleic acid sequence construct can include one or more initiation codons. The initiation codon can be located upstream of the coding sequence. The initiation codon can be in frame with the coding sequence. The initiation codon can be associated with one or more signals required for efficient translation initiation, for example, but not limited to, a ribosome binding site.

Termination Codon

The recombinant nucleic acid sequence construct can include one or more termination or stop codons. The termination codon can be downstream of the coding sequence. The termination codon can be in frame with the coding sequence. The termination codon can be associated with one or more signals required for efficient translation termination.

Polyadenylation Signal

The recombinant nucleic acid sequence construct can include one or more polyadenylation signals. The polyadenylation signal can include one or more signals required for efficient polyadenylation of the transcript. The polyadenylation signal can be positioned downstream of the coding sequence. The polyadenylation signal may be a SV40 polyadenylation signal, LTR polyadenylation signal, bovine growth hormone (bGH) polyadenylation signal, human growth hormone (hGH) polyadenylation signal, or human 3-globin polyadenylation signal. The SV40 polyadenylation signal may be a polyadenylation signal from a pCEP4 plasmid (Invitrogen, San Diego, Calif.).

Leader Sequence

The recombinant nucleic acid sequence construct can include one or more leader sequences. The leader sequence can encode a signal peptide. The signal peptide can be an immunoglobulin (Ig) signal peptide, for example, but not limited to, an IgG signal peptide and a IgE signal peptide.

Expression from the Recombinant Nucleic Acid Sequence Construct

As described above, the recombinant nucleic acid sequence construct can include, amongst the one or more components, the heterologous nucleic acid sequence encoding the heavy chain polypeptide and/or the heterologous nucleic acid sequence encoding the light chain polypeptide. Accordingly, the recombinant nucleic acid sequence construct can facilitate expression of the heavy chain polypeptide and/or the light chain polypeptide.

When arrangement 1 as described above is utilized, the first recombinant nucleic acid sequence construct can facilitate the expression of the heavy chain polypeptide and the second recombinant nucleic acid sequence construct can facilitate expression of the light chain polypeptide. When arrangement 2 as described above is utilized, the recombinant nucleic acid sequence construct can facilitate the expression of the heavy chain polypeptide and the light chain polypeptide.

Upon expression, for example, but not limited to, in a cell, organism, or mammal, the heavy chain polypeptide and the light chain polypeptide can assemble into the synthetic antibody (e.g., DMAb, ScFv antibody fragment, DICE or DBiTE). In particular, the heavy chain polypeptide and the light chain polypeptide can interact with one another such that assembly results in the synthetic antibody (e.g., DMAb, ScFv antibody fragment, DICE or DBiTE) being capable of binding the antigen. In other embodiments, the heavy chain polypeptide and the light chain polypeptide can interact with one another such that assembly results in the synthetic antibody (e.g., DMAb, ScFv antibody fragment, DICE or DBiTE) being more immunogenic as compared to an antibody not assembled as described herein. In still other embodiments, the heavy chain polypeptide and the light chain polypeptide can interact with one another such that assembly results in the synthetic antibody (e.g., DMAb, ScFv antibody fragment, DICE or DBiTE) being capable of eliciting or inducing an immune response against the antigen.

Vector

The recombinant nucleic acid sequence construct described above can be placed in one or more vectors. The one or more vectors can contain an origin of replication. The one or more vectors can be a plasmid, bacteriophage, bacterial artificial chromosome or yeast artificial chromosome. The one or more vectors can be either a self-replication extra chromosomal vector, or a vector which integrates into a host genome.

The one or more vectors can be a heterologous expression construct, which is generally a plasmid that is used to introduce a specific gene into a target cell. Once the expression vector is inside the cell, the heavy chain polypeptide and/or light chain polypeptide that are encoded by the recombinant nucleic acid sequence construct is produced by the cellular-transcription and translation machinery ribosomal complexes. The one or more vectors can express large amounts of stable messenger RNA, and therefore proteins.

Expression Vector

The one or more vectors can be a circular plasmid or a linear nucleic acid. The circular plasmid and linear nucleic acid are capable of directing expression of a particular nucleotide sequence in an appropriate subject cell. The one or more vectors comprising the recombinant nucleic acid sequence construct may be chimeric, meaning that at least one of its components is heterologous with respect to at least one of its other components.

Plasmid

The one or more vectors can be a plasmid. The plasmid may be useful for transfecting cells with the recombinant nucleic acid sequence construct. The plasmid may be useful for introducing the recombinant nucleic acid sequence construct into the subject. The plasmid may also comprise a regulatory sequence, which may be well suited for gene expression in a cell into which the plasmid is administered.

The plasmid may also comprise a mammalian origin of replication in order to maintain the plasmid extrachromosomally and produce multiple copies of the plasmid in a cell. The plasmid may be pVAX1, pCEP4 or pREP4 from Invitrogen (San Diego, Calif.), which may comprise the Epstein Barr virus origin of replication and nuclear antigen EBNA-1 coding region, which may produce high copy episomal replication without integration. The backbone of the plasmid may be pAV0242. The plasmid may be a replication defective adenovirus type 5 (Ad5) plasmid.

The plasmid may be pSE420 (Invitrogen, San Diego, Calif.), which may be used for protein production in Escherichia coli (E. coli). The plasmid may also be p YES2 (Invitrogen, San Diego, Calif.), which may be used for protein production in Saccharomyces cerevisiae strains of yeast. The plasmid may also be of the MAXBAC™ complete baculovirus expression system (Invitrogen, San Diego, Calif.), which may be used for protein production in insect cells. The plasmid may also be pcDNAI or pcDNA3 (Invitrogen, San Diego, Calif.), which may be used for protein production in mammalian cells such as Chinese hamster ovary (CHO) cells.

RNA

In one embodiment, the nucleic acid is an RNA molecule. In one embodiment, the RNA molecule is transcribed from a DNA sequence. Accordingly, in one embodiment, the invention provides an RNA molecule encoding one or more of the synthetic antibodies of the invention. The RNA may be plus-stranded. Accordingly, in some embodiments, the RNA molecule can be translated by cells without needing any intervening replication steps such as reverse transcription. A RNA molecule useful with the invention may have a 5′ cap (e.g. a 7-methylguanosine). This cap can enhance in vivo translation of the RNA. The 5′ nucleotide of a RNA molecule useful with the invention may have a 5′ triphosphate group. In a capped RNA this may be linked to a 7-methylguanosine via a 5′-to-5′ bridge. A RNA molecule may have a 3′ poly-A tail. It may also include a poly-A polymerase recognition sequence (e.g. AAUAAA) near its 3′ end. A RNA molecule useful with the invention may be single-stranded. A RNA molecule useful with the invention may comprise synthetic RNA. In some embodiments, the RNA molecule is a naked RNA molecule. In one embodiment, the RNA molecule is comprised within a vector.

In one embodiment, the RNA has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of RNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many RNAs is known in the art. In other embodiments, the 5′ UTR can be derived from an RNA virus whose RNA genome is stable in cells. In other embodiments, various nucleotide analogues can be used in the 3′ or 5′ UTR to impede exonuclease degradation of the RNA.

In one embodiment, the RNA has both a cap on the 5′ end and a 3′ poly(A) tail which determine ribosome binding, initiation of translation and stability of RNA in the cell.

In one embodiment, the RNA is a nucleoside-modified RNA. Nucleoside-modified RNA have particular advantages over non-modified RNA, including for example, increased stability, low or absent innate immunogenicity, and enhanced translation.

Circular and Linear Vector

The one or more vectors may be circular plasmid, which may transform a target cell by integration into the cellular genome or exist extrachromosomally (e.g., autonomous replicating plasmid with an origin of replication). The vector can be pVAX, pcDNA3.0, or provax, or any other expression vector capable of expressing the heavy chain polypeptide and/or light chain polypeptide encoded by the recombinant nucleic acid sequence construct.

Also provided herein is a linear nucleic acid, or linear expression cassette (“LEC”), that is capable of being efficiently delivered to a subject via electroporation and expressing the heavy chain polypeptide and/or light chain polypeptide encoded by the recombinant nucleic acid sequence construct. The LEC may be any linear DNA devoid of any phosphate backbone. The LEC may not contain any antibiotic resistance genes and/or a phosphate backbone. The LEC may not contain other nucleic acid sequences unrelated to the desired gene expression.

The LEC may be derived from any plasmid capable of being linearized. The plasmid may be capable of expressing the heavy chain polypeptide and/or light chain polypeptide encoded by the recombinant nucleic acid sequence construct. The plasmid can be pNP (Puerto Rico/34) or pM2 (New Caledonia/99). The plasmid may be WLV009, pVAX, pcDNA3.0, or provax, or any other expression vector capable of expressing the heavy chain polypeptide and/or light chain polypeptide encoded by the recombinant nucleic acid sequence construct.

The LEC can be pcrM2. The LEC can be pcrNP. pcrNP and pcrMR can be derived from pNP (Puerto Rico/34) and pM2 (New Caledonia/99), respectively.

Method of Preparing the Vector

Provided herein is a method for preparing the one or more vectors in which the recombinant nucleic acid sequence construct has been placed. After the final subcloning step, the vector can be used to inoculate a cell culture in a large scale fermentation tank, using known methods in the art.

In other embodiments, after the final subcloning step, the vector can be used with one or more electroporation (EP) devices. The EP devices are described below in more detail.

The one or more vectors can be formulated or manufactured using a combination of known devices and techniques, but preferably they are manufactured using a plasmid manufacturing technique that is described in a licensed, co-pending U.S. provisional application U.S. Ser. No. 60/939,792, which was filed on May 23, 2007. In some examples, the DNA plasmids described herein can be formulated at concentrations greater than or equal to 10 mg/mL. The manufacturing techniques also include or incorporate various devices and protocols that are commonly known to those of ordinary skill in the art, in addition to those described in U.S. Ser. No. 60/939,792, including those described in a licensed patent, U.S. Pat. No. 7,238,522, which issued on Jul. 3, 2007. The above-referenced application and patent, U.S. Ser. No. 60/939,792 and U.S. Pat. No. 7,238,522, respectively, are hereby incorporated in their entirety.

Antibody

In some embodiments, the invention relates to a recombinant nucleic acid sequence encoding an antibody, a fragment thereof, a variant thereof, or a combination thereof. The antibody can bind or react with an antigen, which is described in more detail below. In some embodiments, the antibody is a DNA encoded monoclonal antibody (DMAb), a fragment thereof, or a variant thereof. In some embodiments the fragment is an ScFv fragment. In some embodiments, the antibody is a DNA encoded bispecific T cell engagers (BiTE), a fragment thereof, or a variant thereof.

In some embodiments, the antibody may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region. Proceeding from the N-terminus of a heavy or light chain, these regions are denoted as “CDR1,” “CDR2,” and “CDR3,” respectively. An antigen-binding site, therefore, may include six CDRs, comprising the CDR set from each of a heavy and a light chain V region.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)2 fragment, which comprises both antigen-binding sites. Accordingly, the antibody can be the Fab or F(ab′)2. The Fab can include the heavy chain polypeptide and the light chain polypeptide. The heavy chain polypeptide of the Fab can include the VH region and the CH1 region. The light chain of the Fab can include the VL region and CL region.

The antibody can be an immunoglobulin (Ig). The Ig can be, for example, IgA, IgM, IgD, IgE, and IgG. The immunoglobulin can include the heavy chain polypeptide and the light chain polypeptide. The heavy chain polypeptide of the immunoglobulin can include a VH region, a CH1 region, a hinge region, a CH2 region, and a CH3 region. The light chain polypeptide of the immunoglobulin can include a VL region and CL region.

The antibody can be a polyclonal or monoclonal antibody. The antibody can be a chimeric antibody, a single chain antibody, an affinity matured antibody, a human antibody, a humanized antibody, or a fully human antibody. The humanized antibody can be an antibody from a non-human species that binds the desired antigen having one or more complementarity determining regions (CDRs) from the non-human species and framework regions from a human immunoglobulin molecule.

The antibody can be a bispecific antibody as described below in more detail. The antibody can be a bifunctional antibody as also described below in more detail.

As described above, the antibody can be generated in the subject upon administration of the composition to the subject. The antibody may have a half-life within the subject. In some embodiments, the antibody may be modified to extend or shorten its half-life within the subject. Such modifications are described below in more detail.

The antibody can be defucosylated as described in more detail below.

ScFv Antibody

In one embodiment, the DMAb of the invention is a ScFv DMAb. In one embodiment, ScFv DMAb relates to a Fab fragment without the of CH1 and CL regions. Thus, in one embodiment, the ScFv DMAb relates to a Fab fragment DMAb comprising the VH and VL. In one embodiment, the ScFv DMAb comprises a linker between VH and VL. In one embodiment, the ScFv DMAb is an ScFv-Fc DMAb. In one embodiment, the ScFv-Fc DMAb comprises the VH, VL and the CH2 and CH3 regions. In one embodiment, the ScFv-Fc DMAb comprises a linker between VH and VL. In one embodiment, the ScFv DMAb of the invention has modified expression, stability, half-life, antigen binding, heavy chain-light chain pairing, tissue penetration or a combination thereof as compared to a parental DMAb.

In one embodiment, the ScFv DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold higher expression than the parental DMAb.

In one embodiment, the ScFv DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold higher antigen binding than the parental DMAb.

In one embodiment, the ScFv DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold longer half-life than the parental DMAb.

In one embodiment, the ScFv DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold higher stability than the parental DMAb.

In one embodiment, the ScFv DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold greater tissue penetration than the parental DMAb.

In one embodiment, the ScFv DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold greater heavy chain-light chain pairing than the parental DMAb.

In one embodiment, the anti-HER2 scFv antibody comprises an amino acid sequence at least 90% homologous to the amino acid sequence of SEQ ID NO: 66, or a fragment of an amino acid sequence at least 90% homologous to the amino acid sequence of SEQ ID NO: 66. In one embodiment, the anti-HER2 scFv antibody comprises the amino acid of SEQ ID NO: 66, or a fragment of the amino acid sequence of SEQ ID NO: 66. In one embodiment, the anti-HER2 scFv antibody comprises an amino acid sequence at least 90% homologous to the amino acid sequence encoded by SEQ ID NO: 65, or a fragment of an amino acid sequence at least 90% homologous to the amino acid sequence encoded by one of SEQ ID NO: 65. In one embodiment, the anti-HER2 scFv antibody comprises the amino acid sequence encoded by SEQ ID NO: 65, or a fragment of the amino acid sequence encoded by SEQ ID NO: 65.

Monoclonal Antibodies

In one embodiment, the invention provides anti-HER2 antibodies. The antibodies may be intact monoclonal antibodies, and immunologically active fragments (e.g., a Fab or (Fab)₂ fragment), a monoclonal antibody heavy chain, or a monoclonal antibody light chain.

The antibody may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region. Proceeding from the N-terminus of a heavy or light chain, these regions are denoted as “CDR1,” “CDR2,” and “CDR3,” respectively. An antigen-binding site, therefore, may include six CDRs, comprising the CDR set from each of a heavy and a light chain V region.

The antibody can be an immunoglobulin (Ig). The Ig can be, for example, IgA, IgM, IgD, IgE, and IgG. The immunoglobulin can include the heavy chain polypeptide and the light chain polypeptide. The heavy chain polypeptide of the immunoglobulin can include a VH region, a CH1 region, a hinge region, a CH2 region, and a CH3 region. The light chain polypeptide of the immunoglobulin can include a VL region and CL region.

In one embodiment, the anti-HER2 antibody is optimized for expression in human. In one embodiment, the anti-HER2 antibody comprises an amino acid sequence at least 90% homologous to the amino acid sequence of SEQ ID NO: 62, or a fragment of an amino acid sequence at least 90% homologous to the amino acid sequence of SEQ ID NO: 62. In one embodiment, the anti-HER2 antibody comprises the amino acid of SEQ ID NO: 62, or a fragment of the amino acid sequence of SEQ ID NO: 62. In one embodiment, the anti-HER2 antibody comprises an amino acid sequence at least 90% homologous to the amino acid sequence encoded by SEQ ID NO: 61, or a fragment of an amino acid sequence at least 90% homologous to the amino acid sequence encoded by one of SEQ ID NO: 61. In one embodiment, the anti-HER2 antibody comprises the amino acid sequence encoded by SEQ ID NO: 61, or a fragment of the amino acid sequence encoded by SEQ ID NO: 61.

In one embodiment, the anti-HER2 antibody is optimized for expression in mouse. In one embodiment, the anti-HER2 antibody comprises an amino acid sequence at least 90% homologous to the amino acid sequence of SEQ ID NO: 64, or a fragment of an amino acid sequence at least 90% homologous to the amino acid sequence of SEQ ID NO: 64. In one embodiment, the anti-HER2 antibody comprises the amino acid of SEQ ID NO: 64, or a fragment of the amino acid sequence of SEQ ID NO: 64. In one embodiment, the anti-HER2 antibody comprises an amino acid sequence at least 90% homologous to the amino acid sequence encoded by SEQ ID NO: 61, or a fragment of an amino acid sequence at least 90% homologous to the amino acid sequence encoded by one of SEQ ID NO: 63. In one embodiment, the anti-HER2 antibody comprises the amino acid sequence encoded by SEQ ID NO: 63, or a fragment of the amino acid sequence encoded by SEQ ID NO: 63.

Bispecific T Cell Engager

As described above, the recombinant nucleic acid sequence can encode a bispecific T cell engager (BiTE), a fragment thereof, a variant thereof, or a combination thereof. The antigen targeting domain of the BiTE can bind or react with the antigen, which is described in more detail below.

The antigen targeting domain of the BiTE may comprise an antibody, a fragment thereof, a variant thereof, or a combination thereof. The antigen targeting domain of the BiTE may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region. Proceeding from the N-terminus of a heavy or light chain, these regions are denoted as “CDR1,” “CDR2,” and “CDR3,” respectively. An antigen-binding domain, therefore, may include six CDRs, comprising the CDR set from each of a heavy and a light chain V region.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)2 fragment, which comprises both antigen-binding sites. Accordingly, the antigen targeting domain of the BiTE can be the Fab or F(ab′)2. The Fab can include the heavy chain polypeptide and the light chain polypeptide. The heavy chain polypeptide of the Fab can include the VH region and the CH1 region. The light chain of the Fab can include the VL region and CL region.

The antigen targeting domain of the BiTE can be an immunoglobulin (Ig). The Ig can be, for example, IgA, IgM, IgD, IgE, and IgG. The immunoglobulin can include the heavy chain polypeptide and the light chain polypeptide. The heavy chain polypeptide of the immunoglobulin can include a VH region, a CH1 region, a hinge region, a CH2 region, and a CH3 region. The light chain polypeptide of the immunoglobulin can include a VL region and CL region.

The antigen targeting domain of the BiTE can be a polyclonal or monoclonal antibody. The antibody can be a chimeric antibody, a single chain antibody, an affinity matured antibody, a human antibody, a humanized antibody, or a fully human antibody. The humanized antibody can be an antibody from a non-human species that binds the desired antigen having one or more complementarity determining regions (CDRs) from the non-human species and framework regions from a human immunoglobulin molecule.

In one embodiment, at least one of the antigen binding domaing and the immune cell engaging domain of the DBiTE of the invention is a ScFv DNA encoded monoclonal antibody (ScFv DMAb) as described in detail above.

Bispecific Antibody

The recombinant nucleic acid sequence can encode a bispecific antibody, a fragment thereof, a variant thereof, or a combination thereof. The bispecific antibody can bind or react with two antigens, for example, two of the antigens described below in more detail. The bispecific antibody can be comprised of fragments of two of the antibodies described herein, thereby allowing the bispecific antibody to bind or react with two desired target molecules, which may include the antigen, which is described below in more detail, a ligand, including a ligand for a receptor, a receptor, including a ligand-binding site on the receptor, a ligand-receptor complex, and a marker.

The invention provides novel bispecific antibodies comprising a first antigen-binding site that specifically binds to a first target and a second antigen-binding site that specifically binds to a second target, with particularly advantageous properties such as producibility, stability, binding affinity, biological activity, specific targeting of certain T cells, targeting efficiency and reduced toxicity. In some instances, there are bispecific antibodies, wherein the bispecific antibody binds to the first target with high affinity and to the second target with low affinity. In other instances, there are bispecific antibodies, wherein the bispecific antibody binds to the first target with low affinity and to the second target with high affinity. In other instances, there are bispecific antibodies, wherein the bispecific antibody binds to the first target with a desired affinity and to the second target with a desired affinity.

In one embodiment, the bispecific antibody is a bivalent antibody comprising a) a first light chain and a first heavy chain of an antibody specifically binding to a first antigen, and b) a second light chain and a second heavy chain of an antibody specifically binding to a second antigen.

A bispecific antibody molecule according to the invention may have two binding sites of any desired specificity. In some embodiments, one of the binding sites is capable of an tumor antigen. In some embodiments, the binding site included in the Fab fragment is a binding site specific for a tumor antigen. In some embodiments, the binding site included in the single chain Fv fragment is a binding site specific for a tumor antigen such as CD19, BCMA, CD33, FAP, FSHR, EGFR, PSMA, CD123 or Her2.

In some embodiments, one of the binding sites of an antibody molecule according to the invention is able to bind a T-cell specific receptor molecule and/or a natural killer cell (NK cell) specific receptor molecule. A T-cell specific receptor is the so called “T-cell receptor” (TCRs), which allows a T cell to bind to and, if additional signals are present, to be activated by and respond to an epitope/antigen presented by another cell called the antigen-presenting cell or APC. The T cell receptor is known to resemble a Fab fragment of a naturally occurring immunoglobulin. It is generally monovalent, encompassing .alpha.- and .beta.-chains, in some embodiments, it encompasses .gamma.-chains and .delta.-chains (supra). Accordingly, in some embodiments, the TCR is TCR (alpha/beta) and in some embodiments, it is TCR (gamma/delta). The T cell receptor forms a complex with the CD3 T-Cell co-receptor. CD3 is a protein complex and is composed of four distinct chains. In mammals, the complex contains a CD3γ chain, a CD36 chain, and two CD3E chains. These chains associate with a molecule known as the T cell receptor (TCR) and the ζ-chain to generate an activation signal in T lymphocytes. Hence, in some embodiments, a T-cell specific receptor is the CD3 T-Cell co-receptor. In some embodiments, a T-cell specific receptor is CD28, a protein that is also expressed on T cells. CD28 can provide co-stimulatory signals, which are required for T cell activation. CD28 plays important roles in T-cell proliferation and survival, cytokine production, and T-helper type-2 development. Yet a further example of a T-cell specific receptor is CD134, also termed Ox40. CD134/OX40 is being expressed after 24 to 72 hours following activation and can be taken to define a secondary costimulatory molecule. Another example of a T-cell receptor is 4-1 BB capable of binding to 4-1 BB-Ligand on antigen presenting cells (APCs), whereby a costimulatory signal for the T cell is generated. Another example of a receptor predominantly found on T-cells is CD5, which is also found on B cells at low levels. A further example of a receptor modifying T cell functions is CD95, also known as the Fas receptor, which mediates apoptotic signaling by Fas-ligand expressed on the surface of other cells. CD95 has been reported to modulate TCR/CD3-driven signaling pathways in resting T lymphocytes.

An example of a NK cell specific receptor molecule is CD16, a low affinity Fc receptor and NKG2D. An example of a receptor molecule that is present on the surface of both T cells and natural killer (NK) cells is CD2 and further members of the CD2-superfamily. CD2 is able to act as a co-stimulatory molecule on T and NK cells.

In some embodiments, the first binding site of the antibody molecule binds a tumor antigen and the second binding site binds a T cell specific receptor molecule and/or a natural killer (NK) cell specific receptor molecule.

In some embodiments, the first binding site of the antibody molecule binds CD19, BCMA, CD33, FAP, FSHR, EGFR, PSMA, CD123 or Her2, and the second binding site binds a T cell specific receptor molecule and/or a natural killer (NK) cell specific receptor molecule. In some embodiments, the first binding site of the antibody molecule binds CD19, BCMA, CD33, FAP, FSHR, EGFR, PSMA, CD123 or Her2 and the second binding site binds one of CD3, TCR, CD28, CD16, NKG2D, Ox40, 4-1BB, CD2, CD5, CD40, FcgRs, FceRs, FcaRs and CD95. In some embodiments, the first binding site of the antibody molecule binds CD19, BCMA, CD33, FAP, FSHR, EGFR, PSMA, CD123 or Her2 and the second binding site binds CD3.

In some embodiments, the first binding site of the antibody molecule binds a T cell specific receptor molecule and/or a natural killer (NK) cell specific receptor molecule and the second binding site binds a tumor antigen. In some embodiments, the first binding site of the antibody binds a T cell specific receptor molecule and/or a natural killer (NK) cell specific receptor molecule and the second binding site binds CD19, BCMA, CD33, FAP, FSHR, EGFR, PSMA, CD123 or Her2. In some embodiments, the first binding site of the antibody binds one of CD3, TCR, CD28, CD16, NKG2D, Ox40, 4-1BB, CD2, CD5, CD40, FcgRs, FceRs, FcaRs and CD95, and the second binding site binds CD19, BCMA, CD33, FAP, FSHR, EGFR, PSMA, CD123 or Her2. In some embodiments, the first binding site of the antibody binds CD3, and the second binding site binds CD19, BCMA, CD33, FAP, FSHR, EGFR, PSMA, CD123 or Her2.

In one embodiment the bispecific antibody of the invention comprises a DBiTE, comprising one or more scFv antibody fragments as described herein, thereby allowing the DBiTE to bind or react with the desired target molecules.

In one embodiment the DBiTE, comprises a nucleic acid molecule encoding a first scFv specific for binding to a target disease-specific antigen linked to a second scFv specific for binding to a T cell specific receptor molecule. The linkage may place the first and second domains in any order, for example, in one embodiment, a nucleotide sequence encoding a scFv specific for binding to a target disease-specific antigen is oriented 5′ (or upstream) to a nucleotide sequence encoding a scFv specific for binding to a T cell specific receptor molecule. In another embodiment, a nucleotide sequence encoding a scFv specific for binding to a target disease-specific antigen is oriented 3′ (or downstream) to a nucleotide sequence encoding a scFv specific for binding to a T cell specific receptor molecule.

Bifunctional Antibody

The recombinant nucleic acid sequence can encode a bifunctional antibody, a fragment thereof, a variant thereof, or a combination thereof. The bifunctional antibody can bind or react with the antigen described below. The bifunctional antibody can also be modified to impart an additional functionality to the antibody beyond recognition of and binding to the antigen. Such a modification can include, but is not limited to, coupling to factor H or a fragment thereof. Factor H is a soluble regulator of complement activation and thus, may contribute to an immune response via complement-mediated lysis (CML).

Extension of Antibody Half-Life

As described above, the synthetic antibody (e.g., DMAb, ScFv antibody fragment, DICE or DBiTE) may be modified to extend or shorten the half-life of the antibody in the subject. The modification may extend or shorten the half-life of the antibody in the serum of the subject.

The modification may be present in a constant region of the antibody. The modification may be one or more amino acid substitutions in a constant region of the antibody that extend the half-life of the antibody as compared to a half-life of an antibody not containing the one or more amino acid substitutions. The modification may be one or more amino acid substitutions in the CH2 domain of the antibody that extend the half-life of the antibody as compared to a half-life of an antibody not containing the one or more amino acid substitutions.

In some embodiments, the one or more amino acid substitutions in the constant region may include replacing a methionine residue in the constant region with a tyrosine residue, a serine residue in the constant region with a threonine residue, a threonine residue in the constant region with a glutamate residue, or any combination thereof, thereby extending the half-life of the antibody.

In other embodiments, the one or more amino acid substitutions in the constant region may include replacing a methionine residue in the CH2 domain with a tyrosine residue, a serine residue in the CH2 domain with a threonine residue, a threonine residue in the CH2 domain with a glutamate residue, or any combination thereof, thereby extending the half-life of the antibody.

Defucosylation

The recombinant nucleic acid sequence can encode an antibody that is not fucosylated (i.e., a defucosylated antibody or a non-fucosylated antibody), a fragment thereof, a variant thereof, or a combination thereof. Fucosylation includes the addition of the sugar fucose to a molecule, for example, the attachment of fucose to N-glycans, 0-glycans and glycolipids. Accordingly, in a defucosylated antibody, fucose is not attached to the carbohydrate chains of the constant region. In turn, this lack of fucosylation may improve FcγRIIIa binding and antibody directed cellular cytotoxic (ADCC) activity by the antibody as compared to the fucosylated antibody. Therefore, in some embodiments, the non-fucosylated antibody may exhibit increased ADCC activity as compared to the fucosylated antibody.

The antibody may be modified so as to prevent or inhibit fucosylation of the antibody. In some embodiments, such a modified antibody may exhibit increased ADCC activity as compared to the unmodified antibody. The modification may be in the heavy chain, light chain, or a combination thereof. The modification may be one or more amino acid substitutions in the heavy chain, one or more amino acid substitutions in the light chain, or a combination thereof.

Antigen

In one embodiment, the synthetic antibody (e.g., DMAb, ScFv antibody fragment, DICE or DBiTE) is directed to an antigen or fragment or variant thereof. The antigen can be a nucleic acid sequence, an amino acid sequence, a polysaccharide or a combination thereof. The nucleic acid sequence can be DNA, RNA, cDNA, a variant thereof, a fragment thereof, or a combination thereof. The amino acid sequence can be a protein, a peptide, a variant thereof, a fragment thereof, or a combination thereof. The polysaccharide can be a nucleic acid encoded polysaccharide.

The antigen can be a tumor antigen. The antigen can be associated with increased risk of cancer development or progression. In one embodiment, the antigen can be CD19, BCMA, CD33, FAP, FSHR, EGFR, PSMA, CD123 or Her2.

In one embodiment, a synthetic DNA encoded bispecific immune cell engager of the invention targets two or more antigens. In one embodiment, at least one antigen of a bispecific antibody is a tumor antigen. In one embodiment, at least one antigen of a bispecific antibody is a T-cell activating antigen.

Tumor Antigen

The antigen binding domain of the synthetic antibody (e.g., DMAb, ScFv antibody fragment, DICE or DBiTE) of the invention can interact with a tumor antigen. In the context of the present invention, “tumor antigen” or “hyperproliferative disorder antigen” or “antigen associated with a hyperproliferative disorder,” refers to antigens that are common to specific hyperproliferative disorders such as cancer.

The type of tumor antigen referred to in the invention may be a tumor-specific antigen (TSA) or a tumor-associated antigen (TAA). A TSA is unique to tumor cells and does not occur on other cells in the body. A TAA antigen is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

The antigens discussed herein are merely included by way of example. The list is not intended to be exclusive and further examples will be readily apparent to those of skill in the art.

Tumor antigens are proteins that are produced by tumor cells that elicit an immune response, particularly T-cell mediated immune responses. The selection of the antigen binding moiety of the invention will depend on the particular type of cancer to be treated. Tumor antigens are well known in the art and include, for example, a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

Illustrative examples of a tumor associated surface antigen are CD10, CD19, CD20, CD22, CD33, CD123, B-cell maturation antigen (BCMA), Fms-like tyrosine kinase 3 (FLT-3, CD135), chondroitin sulfate proteoglycan 4 (CSPG4, melanoma-associated chondroitin sulfate proteoglycan), Epidermal growth factor receptor (EGFR), Her2, Her3, IGFR, CD133, IL3R, fibroblast activating protein (FAP), CDCP1, Derlin1, Tenascin, frizzled 1-10, the vascular antigens VEGFR2 (KDR/FLK1), VEGFR3 (FLT4, CD309), PDGFR-α (CD140a), PDGFR-.beta. (CD140b) Endoglin, CLEC14, Tem1-8, and Tie2. Further examples may include A33, CAMPATH-1 (CDw52), Carcinoembryonic antigen (CEA), Carboanhydrase IX (MN/CA IX), CD21, CD25, CD30, CD34, CD37, CD44v6, CD45, CD133, de2-7 EGFR, EGFRvIII, EpCAM, Ep-CAM, Folate-binding protein, G250, Fms-like tyrosine kinase 3 (FLT-3, CD135), follicle stimulating hormone receptor (FSHR), c-Kit (CD117), CSF1R (CD115), HLA-DR, IGFR, IL-2 receptor, IL3R, MCSP (Melanoma-associated cell surface chondroitin sulphate proteoglycane), Muc-1, Prostate-specific membrane antigen (PSMA), Prostate stem cell antigen (PSCA), Prostate specific antigen (PSA), and TAG-72. Examples of antigens expressed on the extracellular matrix of tumors are tenascin and the fibroblast activating protein (FAP).

In one embodiment, the tumor antigen is a hormone or fragment thereof which can be used to target a specific receptor. Examples include, but are not limited to, FSH hormone, LH hormone, TSH hormone or fragments thereof.

Non-limiting examples of TSA or TAA antigens include the following: Differentiation antigens such as MART-1/MelanA (MART-I), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

Aspects of the present invention include compositions for enhancing an immune response against an antigen in a subject in need thereof, comprising a synthetic antibody (e.g., DMAb, ScFv antibody fragment, DICE or DBiTE) capable of generating an immune response in the subject, or a biologically functional fragment or variant thereof. In some embodiments, the antigen is CD19, BCMA, CD33, FAP, FSHR, EGFR, PSMA, CD123 or Her2. In some embodiments, the synthetic antibody of this invention is a DBiTE comprising an scFv targeting CD19, BCMA, CD33, FAP, FSHR, EGFR, PSMA, CD123 or Her2.

T Cell Specific Receptor

In one embodiment, the DBiTE or DICE of the invention comprises a scFv of a T cell specific receptor. T cell specific receptors include, but are not limited to, CD3, TCR, CD28, CD16, NKG2D, Ox40, 4-1BB, CD2, CD5, CD40, FcgRs, FceRs, FcaRs and CD95.

Excipients and Other Components of the Composition

The composition may further comprise a pharmaceutically acceptable excipient. The pharmaceutically acceptable excipient can be functional molecules such as vehicles, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and the poly-L-glutamate may be present in the composition at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the composition. The composition may also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. The transfection facilitating agent is a polyanion, polycation, including poly-L-glutamate (LGS), or lipid. Concentration of the transfection agent in the composition is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The composition may further comprise a genetic facilitator agent as described in U.S. Ser. No. 021,579 filed Apr. 1, 1994, which is fully incorporated by reference.

The composition may comprise DNA at quantities of from about 1 nanogram to 100 milligrams; about 1 microgram to about 10 milligrams; or preferably about 0.1 microgram to about 10 milligrams; or more preferably about 1 milligram to about 2 milligram. In some preferred embodiments, composition according to the present invention comprises about 5 nanogram to about 1000 micrograms of DNA. In some preferred embodiments, composition can contain about 10 nanograms to about 800 micrograms of DNA. In some preferred embodiments, the composition can contain about 0.1 to about 500 micrograms of DNA. In some preferred embodiments, the composition can contain about 1 to about 350 micrograms of DNA. In some preferred embodiments, the composition can contain about 25 to about 250 micrograms, from about 100 to about 200 microgram, from about 1 nanogram to 100 milligrams; from about 1 microgram to about 10 milligrams; from about 0.1 microgram to about 10 milligrams; from about 1 milligram to about 2 milligram, from about 5 nanogram to about 1000 micrograms, from about 10 nanograms to about 800 micrograms, from about 0.1 to about 500 micrograms, from about 1 to about 350 micrograms, from about 25 to about 250 micrograms, from about 100 to about 200 microgram of DNA.

The composition can be formulated according to the mode of administration to be used. An injectable pharmaceutical composition can be sterile, pyrogen free and particulate free. An isotonic formulation or solution can be used. Additives for isotonicity can include sodium chloride, dextrose, mannitol, sorbitol, and lactose. The composition can comprise a vasoconstriction agent. The isotonic solutions can include phosphate buffered saline. The composition can further comprise stabilizers including gelatin and albumin. The stabilizers can allow the formulation to be stable at room or ambient temperature for extended periods of time, including LGS or polycations or polyanions.

Method of Generating the Synthetic Antibody

The present invention also relates a method of generating the synthetic antibody. The method can include administering the composition to the subject in need thereof by using the method of delivery described in more detail below. Accordingly, the synthetic antibody is generated in the subject or in vivo upon administration of the composition to the subject.

The method can also include introducing the composition into one or more cells, and therefore, the synthetic antibody can be generated or produced in the one or more cells. The method can further include introducing the composition into one or more tissues, for example, but not limited to, skin and muscle, and therefore, the synthetic antibody can be generated or produced in the one or more tissues.

Method of Delivery of the Composition

The present invention also relates to a method of delivering the composition to the subject in need thereof. The method of delivery can include, administering the composition to the subject. Administration can include, but is not limited to, DNA injection with and without in vivo electroporation, liposome mediated delivery, and nanoparticle facilitated delivery.

The mammal receiving delivery of the composition may be human, primate, non-human primate, cow, cattle, sheep, goat, antelope, bison, water buffalo, bison, bovids, deer, hedgehogs, elephants, llama, alpaca, mice, rats, and chicken.

The composition may be administered by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian can readily determine the dosing regimen and route of administration that is most appropriate for a particular animal. The composition may be administered by traditional syringes, needleless injection devices, “microprojectile bombardment gone guns”, or other physical methods such as electroporation (“EP”), “hydrodynamic method”, or ultrasound.

Electroporation

Administration of the composition via electroporation may be accomplished using electroporation devices that can be configured to deliver to a desired tissue of a mammal, a pulse of energy effective to cause reversible pores to form in cell membranes, and preferable the pulse of energy is a constant current similar to a preset current input by a user. The electroporation device may comprise an electroporation component and an electrode assembly or handle assembly. The electroporation component may include and incorporate one or more of the various elements of the electroporation devices, including: controller, current waveform generator, impedance tester, waveform logger, input element, status reporting element, communication port, memory component, power source, and power switch. The electroporation may be accomplished using an in vivo electroporation device, for example CELLECTRA EP system (Inovio Pharmaceuticals, Plymouth Meeting, Pa.) or Elgen electroporator (Inovio Pharmaceuticals, Plymouth Meeting, Pa.) to facilitate transfection of cells by the plasmid.

The electroporation component may function as one element of the electroporation devices, and the other elements are separate elements (or components) in communication with the electroporation component. The electroporation component may function as more than one element of the electroporation devices, which may be in communication with still other elements of the electroporation devices separate from the electroporation component. The elements of the electroporation devices existing as parts of one electromechanical or mechanical device may not limited as the elements can function as one device or as separate elements in communication with one another. The electroporation component may be capable of delivering the pulse of energy that produces the constant current in the desired tissue, and includes a feedback mechanism. The electrode assembly may include an electrode array having a plurality of electrodes in a spatial arrangement, wherein the electrode assembly receives the pulse of energy from the electroporation component and delivers same to the desired tissue through the electrodes. At least one of the plurality of electrodes is neutral during delivery of the pulse of energy and measures impedance in the desired tissue and communicates the impedance to the electroporation component. The feedback mechanism may receive the measured impedance and can adjust the pulse of energy delivered by the electroporation component to maintain the constant current.

A plurality of electrodes may deliver the pulse of energy in a decentralized pattern. The plurality of electrodes may deliver the pulse of energy in the decentralized pattern through the control of the electrodes under a programmed sequence, and the programmed sequence is input by a user to the electroporation component. The programmed sequence may comprise a plurality of pulses delivered in sequence, wherein each pulse of the plurality of pulses is delivered by at least two active electrodes with one neutral electrode that measures impedance, and wherein a subsequent pulse of the plurality of pulses is delivered by a different one of at least two active electrodes with one neutral electrode that measures impedance.

The feedback mechanism may be performed by either hardware or software. The feedback mechanism may be performed by an analog closed-loop circuit. The feedback occurs every 50 μs, 20 μs, 10 μs or 1 μs, but is preferably a real-time feedback or instantaneous (i.e., substantially instantaneous as determined by available techniques for determining response time). The neutral electrode may measure the impedance in the desired tissue and communicates the impedance to the feedback mechanism, and the feedback mechanism responds to the impedance and adjusts the pulse of energy to maintain the constant current at a value similar to the preset current. The feedback mechanism may maintain the constant current continuously and instantaneously during the delivery of the pulse of energy.

Examples of electroporation devices and electroporation methods that may facilitate delivery of the composition of the present invention, include those described in U.S. Pat. No. 7,245,963 by Draghia-Akli, et al., U.S. Patent Pub. 2005/0052630 submitted by Smith, et al., the contents of which are hereby incorporated by reference in their entirety. Other electroporation devices and electroporation methods that may be used for facilitating delivery of the composition include those provided in co-pending and co-owned U.S. patent application Ser. No. 11/874,072, filed Oct. 17, 2007, which claims the benefit under 35 USC 119(e) to U.S. Provisional Application Ser. No. 60/852,149, filed Oct. 17, 2006, and 60/978,982, filed Oct. 10, 2007, all of which are hereby incorporated in their entirety.

U.S. Pat. No. 7,245,963 by Draghia-Akli, et al. describes modular electrode systems and their use for facilitating the introduction of a biomolecule into cells of a selected tissue in a body or plant. The modular electrode systems may comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The biomolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the biomolecule into the cell between the plurality of electrodes. The entire content of U.S. Pat. No. 7,245,963 is hereby incorporated by reference.

U.S. Patent Pub. 2005/0052630 submitted by Smith, et al. describes an electroporation device which may be used to effectively facilitate the introduction of a biomolecule into cells of a selected tissue in a body or plant. The electroporation device comprises an electro-kinetic device (“EKD device”) whose operation is specified by software or firmware. The EKD device produces a series of programmable constant-current pulse patterns between electrodes in an array based on user control and input of the pulse parameters, and allows the storage and acquisition of current waveform data. The electroporation device also comprises a replaceable electrode disk having an array of needle electrodes, a central injection channel for an injection needle, and a removable guide disk. The entire content of U.S. Patent Pub. 2005/0052630 is hereby incorporated by reference.

The electrode arrays and methods described in U.S. Pat. No. 7,245,963 and U.S. Patent Pub. 2005/0052630 may be adapted for deep penetration into not only tissues such as muscle, but also other tissues or organs. Because of the configuration of the electrode array, the injection needle (to deliver the biomolecule of choice) is also inserted completely into the target organ, and the injection is administered perpendicular to the target issue, in the area that is pre-delineated by the electrodes The electrodes described in U.S. Pat. No. 7,245,963 and U.S. Patent Pub. 2005/005263 are preferably 20 mm long and 21 gauge.

Additionally, contemplated in some embodiments that incorporate electroporation devices and uses thereof, there are electroporation devices that are those described in the following patents: U.S. Pat. No. 5,273,525 issued Dec. 28, 1993, U.S. Pat. No. 6,110,161 issued Aug. 29, 2000, U.S. Pat. No. 6,261,281 issued Jul. 17, 2001, and U.S. Pat. No. 6,958,060 issued Oct. 25, 2005, and U.S. Pat. No. 6,939,862 issued Sep. 6, 2005. Furthermore, patents covering subject matter provided in U.S. Pat. No. 6,697,669 issued Feb. 24, 2004, which concerns delivery of DNA using any of a variety of devices, and U.S. Pat. No. 7,328,064 issued Feb. 5, 2008, drawn to method of injecting DNA are contemplated herein. The above-patents are incorporated by reference in their entirety.

Method of Treatment

Also provided herein is a method of treating, protecting against, and/or preventing disease in a subject in need thereof by generating the synthetic antibody (e.g., DMAb, ScFv fragment or DBiTE) in the subject. The method can include administering the composition to the subject. Administration of the composition to the subject can be done using the method of delivery described above.

In certain embodiments, the invention provides a method of treating protecting against, and/or preventing cancer. In one embodiment, the method treats, protects against, and/or prevents tumor growth. In one embodiment, the method treats, protects against, and/or prevents cancer progression. In one embodiment, the method treats, protects against, and/or prevents cancer metastasis.

In one embodiment, the invention provides methods for preventing growth of benign tumors, such as, but not limited to, uterine fibroids. The methods comprise administering an effective amount of one or more of the compositions of the invention to a subject diagnosed with a benign tumor.

Upon generation of the synthetic antibody (e.g., DMAb, ScFv fragment or DBiTE) in the subject, the synthetic antibody (e.g., DMAb, ScFv fragment or DBiTE) can bind to or react with the antigen. Such binding can neutralize the antigen, block recognition of the antigen by another molecule, for example, a protein or nucleic acid, and elicit or induce an immune response to the antigen, thereby treating, protecting against, and/or preventing the disease associated with the antigen in the subject.

The composition dose can be between 1 μg to 10 mg active component/kg body weight/time, and can be 20 μg to 10 mg component/kg body weight/time. The composition can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of composition doses for effective treatment can be 1, 2, 3, 4, 5, 6, 7, 8, 9, or 10.

Cancer Therapy

The invention provides methods of treating or preventing cancer, or of treating and preventing growth or metastasis of tumors. Related aspects of the invention provide methods of preventing, aiding in the prevention, and/or reducing metastasis of hyperplastic or tumor cells in an individual.

One aspect of the invention provides a method of inhibiting metastasis in an individual in need thereof, the method comprising administering to the individual an effective amount of a composition of the invention. The invention further provides a method of inhibiting metastasis in an individual in need thereof, the method comprising administering to the individual an effective metastasis-inhibiting amount of any one of the compositions described herein.

In some embodiments of treating or preventing cancer, or of treating and preventing metastasis of tumors in an individual in need thereof, a second agent is administered to the individual, such as an antineoplastic agent. In some embodiments, the second agent comprises a second metastasis-inhibiting agent, such as a plasminogen antagonist, or an adenosine deaminase antagonist. In other embodiments, the second agent is an angiogenesis inhibiting agent.

The compositions of the invention can be used to prevent, abate, minimize, control, and/or lessen cancer in humans and animals. The compositions of the invention can also be used to slow the rate of primary tumor growth. The compositions of the invention when administered to a subject in need of treatment can be used to stop the spread of cancer cells. As such, the compositions of the invention can be administered as part of a combination therapy with one or more drugs or other pharmaceutical agents. When used as part of the combination therapy, the decrease in metastasis and reduction in primary tumor growth afforded by the compositions of the invention allows for a more effective and efficient use of any pharmaceutical or drug therapy being used to treat the patient. In addition, control of metastasis by the compositions of the invention affords the subject a greater ability to concentrate the disease in one location.

In one embodiment, the invention provides methods for preventing metastasis of malignant tumors or other cancerous cells as well as to reduce the rate of tumor growth. The methods comprise administering an effective amount of one or more of the compositions of the invention to a subject diagnosed with a malignant tumor or cancerous cells or to a subject having a tumor or cancerous cells.

The following are non-limiting examples of cancers that can be treated by the methods and compositions of the invention: Acute Lymphoblastic; Acute Myeloid Leukemia; Adrenocortical Carcinoma; Adrenocortical Carcinoma, Childhood; Appendix Cancer; Basal Cell Carcinoma; Bile Duct Cancer, Extrahepatic; Bladder Cancer; Bone Cancer; Osteosarcoma and Malignant Fibrous Histiocytoma; Brain Stem Glioma, Childhood; Brain Tumor, Adult; Brain Tumor, Brain Stem Glioma, Childhood; Brain Tumor, Central Nervous System Atypical Teratoid/Rhabdoid Tumor, Childhood; Central Nervous System Embryonal Tumors; Cerebellar Astrocytoma; Cerebral Astrocytotna/Malignant Glioma; Craniopharyngioma; Ependymoblastoma; Ependymoma; Medulloblastoma; Medulloepithelioma; Pineal Parenchymal Tumors of intermediate Differentiation; Supratentorial Primitive Neuroectodermal Tumors and Pineoblastoma; Visual Pathway and Hypothalamic Glioma; Brain and Spinal Cord Tumors; Breast Cancer; Bronchial Tumors; Burkitt Lymphoma; Carcinoid Tumor; Carcinoid Tumor, Gastrointestinal; Central Nervous System Atypical Teratoid/Rhabdoid Tumor; Central Nervous System Embryonal Tumors; Central Nervous System Lymphoma; Cerebellar Astrocytoma Cerebral Astrocytoma/Malignant Glioma, Childhood; Cervical Cancer; Chordoma, Childhood; Chronic Lymphocytic Leukemia; Chronic Myelogenous Leukemia; Chronic Myeloproliferative Disorders; Colon Cancer; Colorectal Cancer; Craniopharyngioma; Cutaneous T-Cell Lymphoma; Esophageal Cancer; Ewing Family of Tumors; Extragonadal Germ Cell Tumor; Extrahepatic Bile Duct Cancer; Eye Cancer, intraocular Melanoma; Eye Cancer, Retinoblastoma; Gallbladder Cancer; Gastric (Stomach) Cancer; Gastrointestinal Carcinoid Tumor; Gastrointestinal Stromal Tumor (GIST); Germ Cell Tumor, Extracranial; Germ Cell Tumor, Extragonadal; Germ Cell Tumor, Ovarian; Gestational Trophoblastic Tumor; Glioma; Glioma, Childhood Brain Stem; Glioma, Childhood Cerebral Astrocytoma; Glioma, Childhood Visual Pathway and Hypothalamic; Hairy Cell Leukemia; Head and Neck Cancer; Hepatocellular (Liver) Cancer; Histiocytosis, Langerhans Cell; Hodgkin Lymphoma; Hypopharyngeal Cancer; Hypothalamic and Visual Pathway Glioma; intraocular Melanoma; Islet Cell Tumors; Kidney (Renal Cell) Cancer; Langerhans Cell Histiocytosis; Laryngeal Cancer; Leukemia, Acute Lymphoblastic; Leukemia, Acute Myeloid; Leukemia, Chronic Lymphocytic; Leukemia, Chronic Myelogenous; Leukemia, Hairy Cell; Lip and Oral Cavity Cancer; Liver Cancer; Lung Cancer, Non-Small Cell; Lung Cancer, Small Cell; Lymphoma, AIDS-Related; Lymphoma, Burkitt; Lymphoma, Cutaneous T-Cell; Lymphoma, Hodgkin; Lymphoma, Non-Hodgkin; Lymphoma, Primary Central Nervous System; Macroglobulinemia, Waldenstrom; Malignant Fibrous Histiocytoma of Bone and Osteosarcoma; Medulloblastoma; Melanoma; Melanoma, intraocular (Eye); Merkel Cell Carcinoma; Mesothelioma; Metastatic Squamous Neck Cancer with Occult Primary; Mouth Cancer; Multiple Endocrine Neoplasia Syndrome, (Childhood); Multiple Myeloma/Plasma Cell Neoplasm; Mycosis; Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple; Myeloproliferative Disorders, Chronic; Nasal Cavity and Paranasal Sinus Cancer; Nasopharyngeal Cancer; Neuroblastoma; Non-Small Cell Lung Cancer; Oral Cancer; Oral Cavity Cancer; Oropharyngeal Cancer; Osteosarcoma and Malignant Fibrous Histiocytoma of Bone; Ovarian Cancer; Ovarian Epithelial Cancer; Ovarian Germ Cell Tumor; Ovarian Low Malignant Potential Tumor; Pancreatic Cancer; Pancreatic Cancer, Islet Cell Tumors; Papillomatosis; Parathyroid Cancer; Penile Cancer; Pharyngeal Cancer; Pheochromocytoma; Pineal Parenchymal Tumors of Intermediate Differentiation; Pineoblastoma and Supratentorial Primitive Neuroectodermal Tumors; Pituitary Tumor; Plasma Celt Neoplasm/Multiple Myeloma; Pleuropulmonary Blastoma; Primary Central Nervous System Lymphoma; Prostate Cancer; Rectal Cancer; Renal Cell (Kidney) Cancer; Renal Pelvis and Ureter, Transitional Cell Cancer; Respiratory Tract Carcinoma Involving the NUT Gene on Chromosome 15; Retinoblastoma; Rhabdomyosarcoma; Salivary Gland Cancer; Sarcoma, Ewing Family of Tumors; Sarcoma, Kaposi; Sarcoma, Soft Tissue; Sarcoma, Uterine; Sezary Syndrome; Skin Cancer (Nonmelanoma); Skin Cancer (Melanoma); Skin Carcinoma, Merkel Cell; Small Cell Lung Cancer; Small Intestine Cancer; Soft Tissue Sarcoma; Squamous Cell Carcinoma, Squamous Neck Cancer with Occult Primary, Metastatic; Stomach (Gastric) Cancer; Supratentorial Primitive Neuroectodermal Tumors; T-Cell Lymphoma, Cutaneous; Testicular Cancer; Throat Cancer; Thymoma and Thymic Carcinoma; Thyroid Cancer; Transitional Cell Cancer of the Renal Pelvis and Ureter; Trophoblastic Tumor, Gestational; Urethral Cancer; Uterine Cancer, Endometrial; Uterine Sarcoma; Vaginal Cancer; Vulvar Cancer; Waldenstrom Macroglobulinemia; and Wilms Tumor.

In one embodiment, the invention provides a method to treat cancer metastasis comprising treating the subject prior to, concurrently with, or subsequently to the treatment with a composition of the invention, with a complementary therapy for the cancer, such as surgery, chemotherapy, chemotherapeutic agent, radiation therapy, or hormonal therapy or a combination thereof.

Chemotherapeutic agents include cytotoxic agents (e.g., 5-fluorouracil, cisplatin, carboplatin, methotrexate, daunorubicin, doxorubicin, vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic alkylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium).

Antiproliferative agents are compounds that decrease the proliferation of cells. Antiproliferative agents include alkylating agents, antimetabolites, enzymes, biological response modifiers, miscellaneous agents, hormones and antagonists, androgen inhibitors (e.g., flutamide and leuprolide acetate), antiestrogens (e.g., tamoxifen citrate and analogs thereof, toremifene, droloxifene and roloxifene), Additional examples of specific antiproliferative agents include, but are not limited to levamisole, gallium nitrate, granisetron, sargramostim strontium-89 chloride, filgrastim, pilocarpine, dexrazoxane, and ondansetron.

The compounds of the invention can be administered alone or in combination with other anti-tumor agents, including cytotoxic/antineoplastic agents and anti-angiogenic agents. Cytotoxic/anti-neoplastic agents are defined as agents which attack and kill cancer cells. Some cytotoxic/anti-neoplastic agents are alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents are antimetabolites for tumor cells, e.g., cytosine arabinoside, fluorouracil, methotrexate, mercaptopuirine, azathioprime, and procarbazine. Other cytotoxic/anti-neoplastic agents are antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents are mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents are well known to those of skill in the art. Suitable anti-angiogenic agents for use in the methods and compositions of the invention include anti-VEGF antibodies, including humanized and chimeric antibodies, anti-VEGF aptamers and antisense oligonucleotides. Other known inhibitors of angiogenesis include angiostatin, endostatin, interferons, interleukin 1 (including alpha and beta) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with the compositions of the invention include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. In one embodiment, the anti-cancer drug is 5-fluorouracil, taxol, or leucovorin.

Generation of Synthetic Antibodies In Vitro and Ex Vivo

In one embodiment, the synthetic antibody (e.g., DMAb, ScFv fragment or DBiTE) is generated in vitro or ex vivo. For example, in one embodiment, a nucleic acid encoding a synthetic antibody (e.g., DMAb, ScFv fragment or DBiTE) can be introduced and expressed in an in vitro or ex vivo cell. Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

### EXAMPLES

The present invention is further illustrated in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1

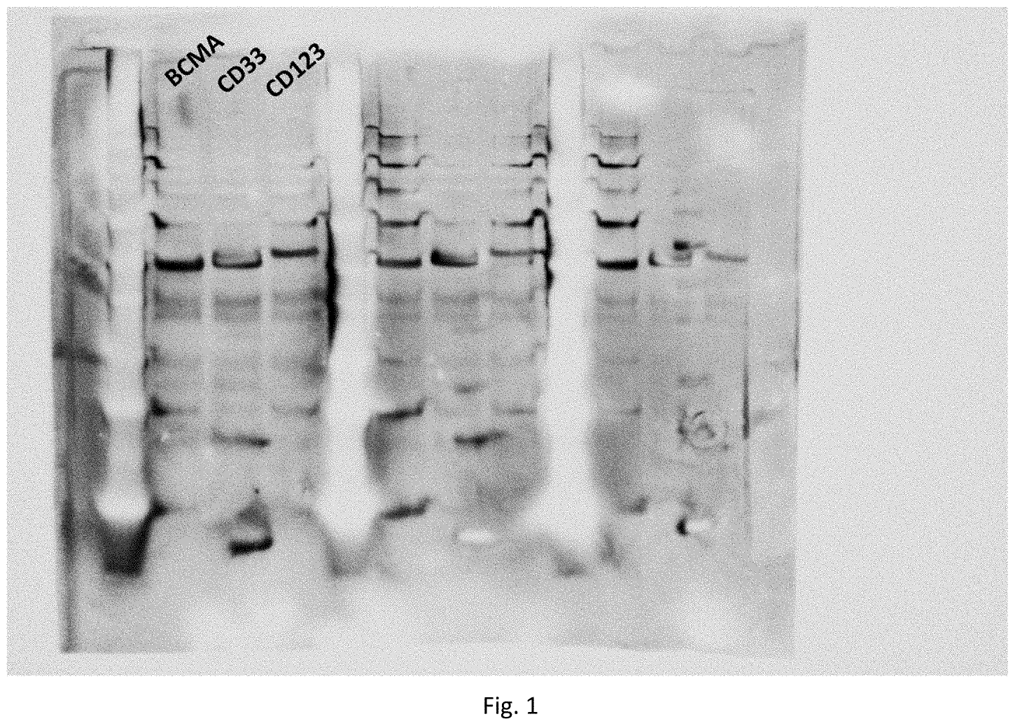

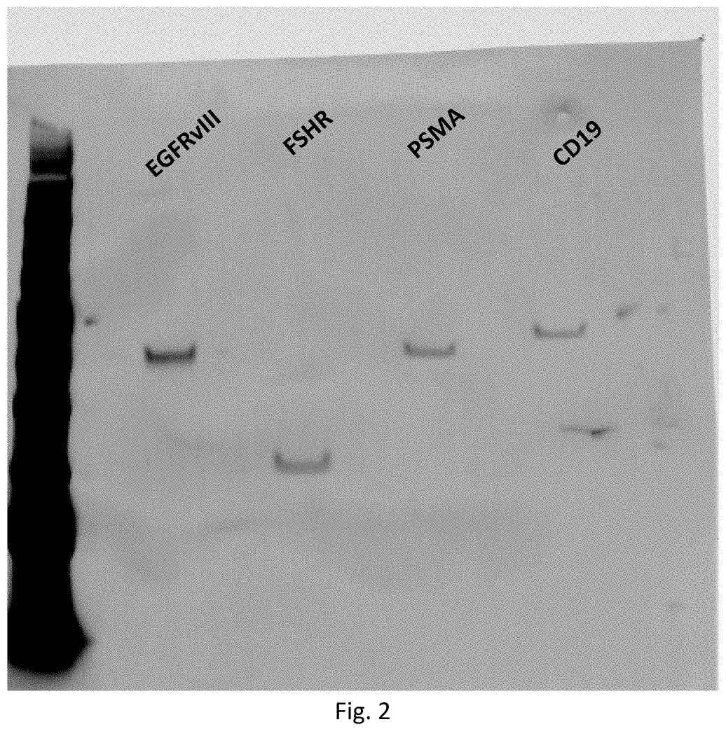

The studies presented herein demonstrate the development of DNA encoded bispecific T-cell engagers (DBiTEs) targeting CD19, BCMA, CD33, FAP, FSHR, EGFR, PSMA, or CD123. The DBiTE constructs express at high levels in vivo. FIG. 1 shows the expression of BCMADBiTE, CD33DBiTE and CD123DBiTE. FIG. 2 shows the expression of EGFRvIIIDBiTE, FSHRDBiTE, PSMADBiTE and CD19DBiTE. These novel DBiTEs represent new tools for immunotherapy of cancer.

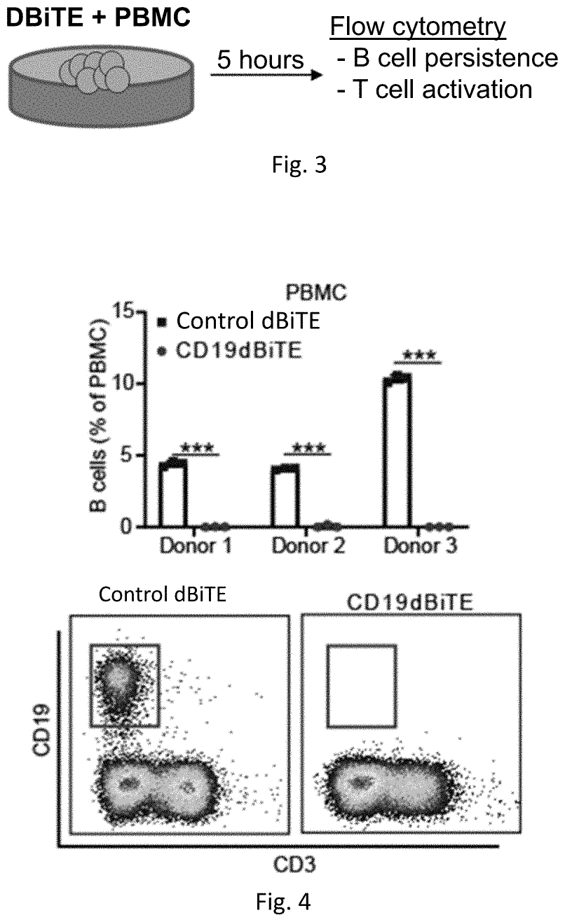

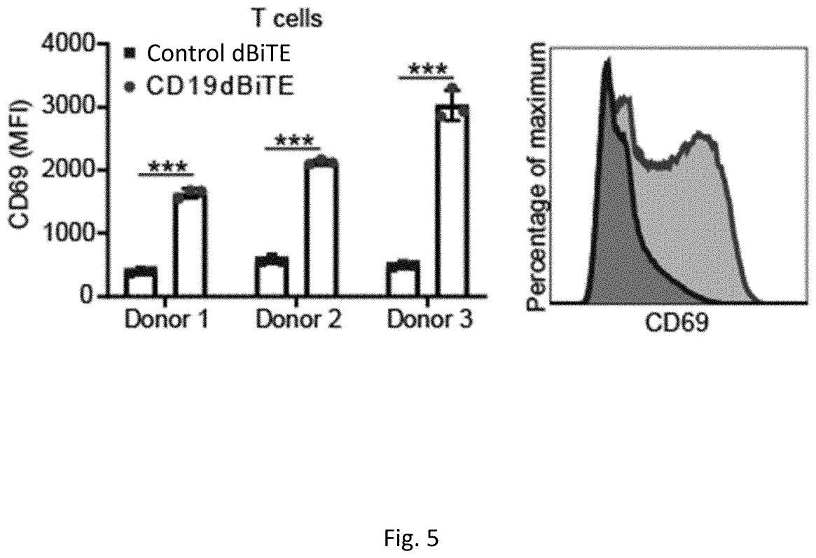

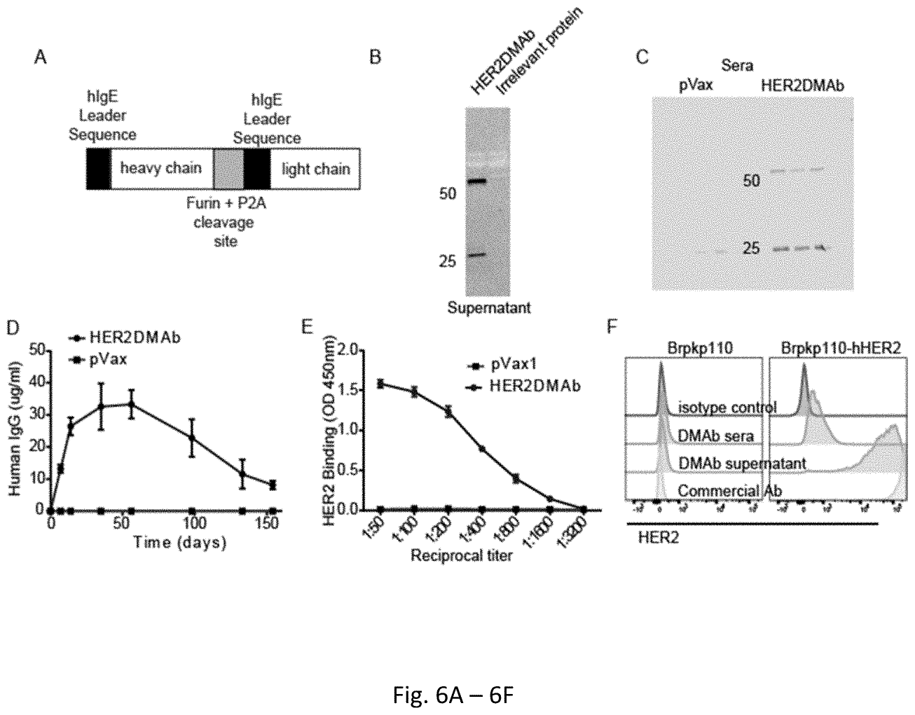

FIGS. 3 through 5 provide data demonstrating that the CD19DBiTE functions for both B cell depletion and T cell activation. PBMC from 3 independent donors were cultured in triplicate for 5 hours in the presence of 5 μl of supernatant of CD19DBiTE or a control DBiTE (EGFRvIIIDBiTE). Following incubation, the cells were stained for B cell and T cell markers to determine the potential cytolytic activity against B cells (CD19+ cells) and T cell early activation. FIG. 4 provides data showing that all three donors presented depletion of their B cells (CD19+ cells in the PBMC mix) in the presence of CD19DBiTE but not in the presence of the control DBiTE. FIG. 5 provides data showing that all three donors presented increase in the early activation marker CD69 in their T cells in the presence of CD19DBiTE but not in the presence of the control DBiTE.

In addition, experiments were performed to demonstrate the cytotoxicity of BCMADBiTE. BCMADBiTE supernatant or CD33DBiTE supernatant was incubated with the RPMI8226 cell line for 5 hours and T cells derived form one donor at 1:0, 1:1, 1:3 and 1:7 tumor to T cell ratios (10,000 tumor cells per well). Upon a 5 hour incubation, BCMAdBiTE was able to lyse cells at the 1:1, 1:3 and 1:7 ratios, but not in the absene of T cells, and no killing occurred in the presence of CD33DBiTE in any condition.

### Example 2

The studies presented herein demonstrate the development of a DNA monoclonal antibody and BiTE targeting HER2 (HER2DMAb & HER2DBiTE) and their use as a therapeutic for the treatment of ovarian and breast cancer. Both the DMAb and DBiTE constructs express at high levels in vitro and in vivo for approximately 4 months. HER2DMAb binds to HER2 and induces HER2 signaling blockade and antibody-dependent cellular cytotoxicity. HER2DBiTE effectively induces T cell cytotoxicity against HER2+ tumor cells. These novel DNA technologies represent new tools for further study for immunotherapy of cancer.

The Material and Methods are now described

Animals and Cell Lines

C57Bl/6 and Nu/J mice were purchased from Jackson labs. NSG mice were purchased from the Wistar Institute Animal Facility.

OVCAR3, SKOV3 and Brpkp110 cells were provided by J. R. Conejo-Garcia (Department of Immunology, Moffitt Cancer Center, FL). TOV-21G and RNG1 were provided by R. Zhang (The Wistar Institute). OVCAR3 tumors were generated by injecting 3 million cells in the flank in PBS/Matrigel (50/50) as described previously (Perales-Puchalt et al., 2017, Clin Cancer Res, 23(2):441-53). RD and 293T cells were purchased from ATCC.

Mice were treated by injecting 100 ug of DNA resuspended in 80 ul of water into the tibialis anterior muscle (40 ul per leg) with 200 IU/ml of hyaluronidase (Sigma) followed a minute after injection by electroporation with the CELLECTRA device.

Design of HER2DMAb and HER2DBiTE

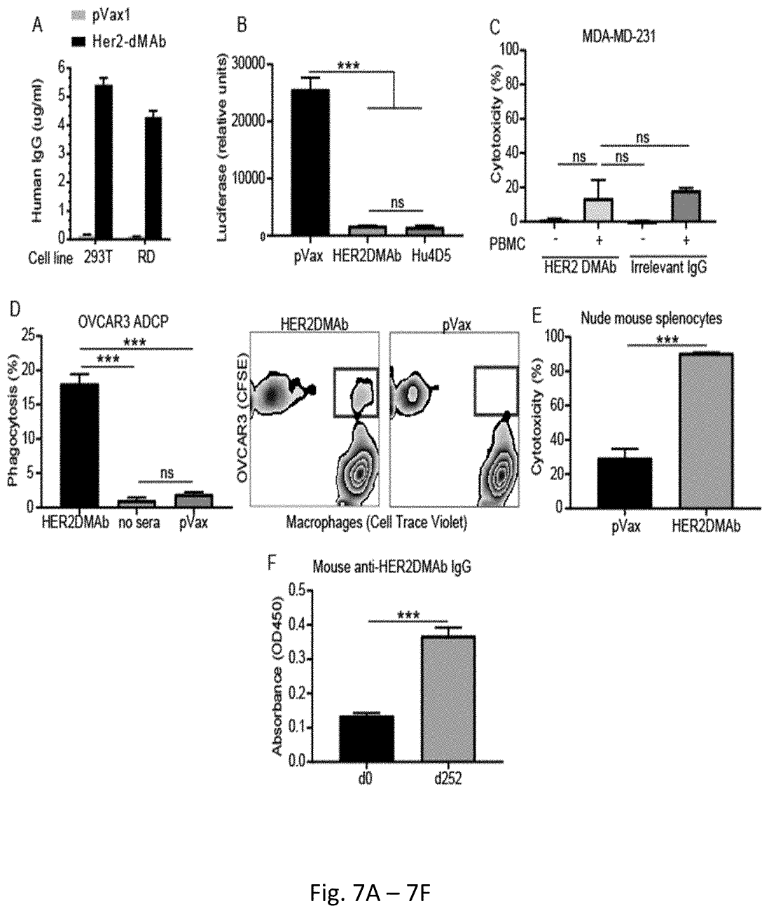

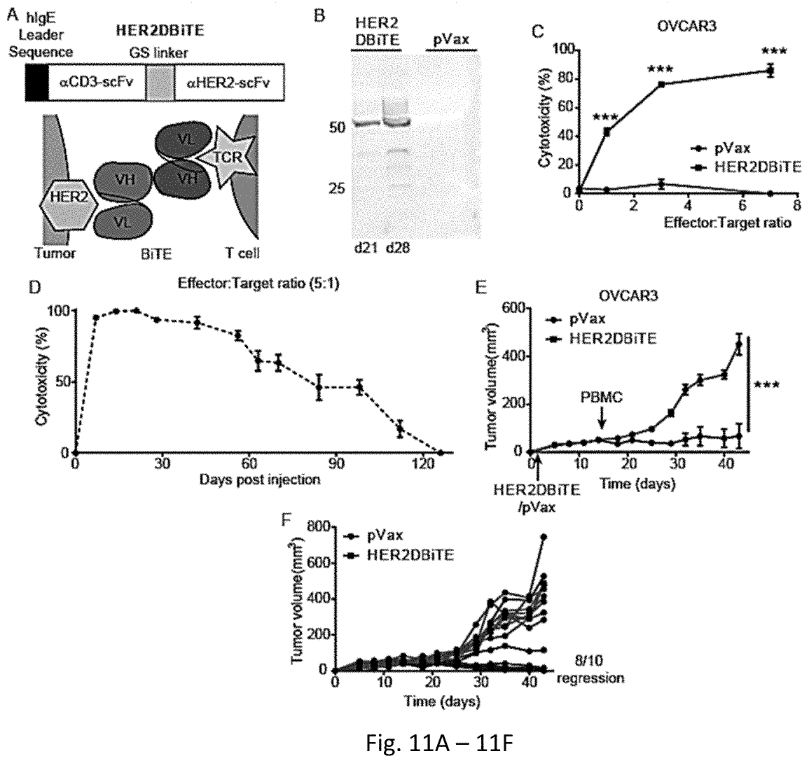

A HER2DMAb was designed and generated encoding a codon optimized sequence of the heavy and light chains of the anti-HER2 monoclonal antibody pertuzumab. Both antibody chains were positioned in sequence separated by a P2A and furin cleavage sites. The IgE leader sequence was substituted for the original leader sequence. A HER2DBiTE was designed by encoding a codon optimized scFv of HER2DMAb followed by the scFv of OKT3 anti-human CD3 antibody and adding an IgE leader sequence. Both constructs were subcloned it into a modified pVAX1 expression vector (FIG. 6A and FIG. 11A).

An empty modified pVAX1 plasmid was used as a negative control.

In Vitro DMAb Expression

1 million 293T cells were plated in each chamber of a 6-well plate. The following day the cells were transfected with 1 μg of HER2DMAb plasmid with Lipofectamin 2000 (Invitrogen). The supernatant was collected 48 hours post transfection.

Flow Cytometry

Anti-human antibodies used were directly fluorochrome conjugated. HER2 (24D2), CD45 (H130), CD3 (HIT3A), CD69 (FN50), PD-1 (EH12.2H7), and secondary anti-human IgG APC (polyclonal) were obtained from Biolegend. Live/dead exclusion was done with 7AAD (Invitrogen) and Annexin V (Biolegend).

Immunoblotting

Protein extraction, denaturation and western blotting were performed as previously described (Perales-Puchalt et al., 2017, Clin Cancer Res, 23(2):441-53). Membranes were blotted with polyclonal anti-human IgG (H+L) (Bethyl) and anti-R-actin (a5441, Sigma-Aldrich). Images were captured with ImageQuantLAS 4000 (GE Healthcare Life Sciences).

For signaling blockade experiments 200,000 OVCAR3 cells were plated in a 6-well plate and starved overnight with serum free media. On the next day, 10 μg of purified HER2DMAb or PBS were added to the appropriate wells for 1 h followed by 10 ng/ml of HRG (Peprotech) for 30 minutes.

HER2 Binding ELISA

ELISA plates were coated with 1 ug/ml of human HER2 recombinant protein (abeam) overnight at 4° C. Blocking was performed with PBST-10% FBS for 1 hour. Sera from HER2DMAb expressing mice or controls (electroporated with empty pVax plasmid) at different dilutions was used as primary antibody, and incubation was performed at room temperature for 1 hour. Secondary antibody was a Goat anti-human IgG Fc HRP conjugated (Bethyl). After 1 hour incubation, development was performed with SIGMAFAST OPD (Sigma Aldrich) and read at 450 nm.

DMAb Quantification ELISA

ELISA plates were coated with 1 μg/ml of goat anti-human IgG-Fc fragment antibody (Bethyl) overnight at 4° C. The following day, they were blocked with PBST-10% FBS for 1 hour at room temperature, washed, incubated for 1 hour at room temperature with the samples diluted in PBST-1% FBS, washed, and incubated at room temperature with HRP conjugated goat anti-human kappa light chain antibody (Bethyl). After 1 hour incubation, they were developed with SIGMAFAST OPD (Sigma Aldrich) and read at 450 nm. The standard curve was generated using purified human IgG/Kappa (Bethyl).

CD3 and HER2 Binding ELISA (DBiTE)

ELISA plates were coated with 1 μg/ml of human HER2 recombinant protein (abcam) or human CD3 epsilon (Acrobiosystems) overnight at 4° C. They were blocked with PBST-10% FBS for 1 hour. Sera from HER2DBiTE expressing mice or controls (electroporated with empty pVax plasmid) was used as primary antibody. They were incubated at room temperature for 1 hour. Secondary antibody was a Goat anti-human IgG H+L HRP conjugated (Bethyl). After 1 hour incubation plates were developed with SIGMAFAST OPD (Sigma Aldrich) and read at 450 nm.

Detection of Anti-HER2DMAb and HER2DBiTE Antibodies

ELISA plates were coated with 1 μg/ml of purified HER2DMAb or HER2DBiTE overnight at 4° C. The following day, plates were blocked with PBST-10% FBS for 1 hour at room temperature, washed, incubated for 1 hour at room temperature with the samples diluted in PBST-1% FBS, washed, and incubated at room temperature with HRP conjugated goat anti-mouse IgG antibody (Abcam). After 1 hour incubation, plates were developed with SIGMAFAST OPD (Sigma Aldrich).

Detection of T Cell Activation and Apoptosis by HER2DBiTE

96-well plates were plated with 5,000 OVCAR3 overnight at 4° C. The following day, sera from HER2DBiTE expressing mice or pVax controls (1:20 dilution in PBS, 100 ul) and 50,000 T cells were added and the plates were incubated at 37° C. 24 hours later, supernatant was taken for IFNγ ELISA and fresh supernatant was added. After 72 hours flow cytometry was performed to measure T cell apoptosis and activation (CD3, CD69, PD-1, Annexin V). For cell counts, 5,000 OVCAR3 were plated with 100,000 T cells and live T cell numbers were counted using dead cell exclusion dye Trypan Blue (ThermoFisher) and Countess II automated cell counter (ThermoFisher).

Interferon gamma ELISA

Determination of human interferon gamma from supernatants was performed using Human IFNg ELISA MAX (Biolegend) following manufacturer's instructions.

In Vitro Cytotoxicity

10,000 OVCAR3 cells per well were plated in a 96-well plate and 18 hours later were coincubated for 4 hours with 500,000 human PBMC from a healthy donor (provided by the University of Pennsylvania Human Immunology Core) or 500,000 splenocytes from nude mice in the presence or absence of HER2DMAb. After 4 hours the supernatant was collected, the cells were trypsinized and stained for 7AAD (Invitrogen), Annexin V (Biolegend) and anti-human CD45 (Biolegend) and a flow cytometry-based cytotoxicity assay was performed as described previously (Perales-Puchalt et al., 2017, Clin Cancer Res, 23(2):441-53). Alternatively, we used OVCAR3 or MDA-MB-231 expressing luciferase and after coculture measured luciferase expression. For BiTE killing assay, 10,000 OVCAR3-luciferase cells were incubated with different ratios of T cells for 5 hours, washed with PBS, lysed and the luciferase expression was measured.

Antibody Dependent Cellular Phagocytosis

Macrophages were differentiated from human monocytes by plating 1 million monocytes per T25 with 50 ng/ml of human M-CSF (Peprotech). The media with cytokines was changed at days 3 and 6. On day 6 the macrophages were trypsinized, stained them with cell trace violet (Invitrogen) according to manufacturer's instruction and plated 50,000/well in a 96-well plate and lefT them with 20 ng/ml M-CSF overnight. On day 7 OVCAR3 cells were stained with CFSE (Invitrogen) and 10,000 OVCAR3 cells were plated on the wells with macrophages with HER2DMAb or pVax sera. 24 hours later the cells were trypsinized, and flow cytometry was performed. Phagocytosis was measured as double positive stained cells.

Immunofluorescence

Mouse tumors were frozen in OCT (TissueTek), and frozen sections cut. Slides were then fixed with 4% paraformaldehyde and permeabilized with 0.5% Triton X-100 in PBS. Sections were blocked using 5% normal goat serum followed by staining with HER2DMAb antibodies and anti-human AF488 conjugated secondary (Invitrogen).

Slides were viewed using the Leica TCS SP5 II confocal microscope and the LAS software (Leica).

Statistics

Differences between the means of experimental groups were calculated using a two-tailed unpaired Student's t test or one-way ANOVA where two categorical variables were measured. Repeated measures were analyzed using 2-way ANOVA. Error bars represent standard error of the mean. Survival rates were compared using the log-rank test. All statistical analyses were done using Graph Pad Prism 7.0. p<0.05 was considered statistically significant.

The results of the Experiments are now described

Design and Expression of HER2 DNA Encoded Monoclonal Antibody (DMAb).

DNA encoded antibodies (DMAb) have a series of advantages over the traditional protein antibodies. Firstly, DNA is more stable than proteins. This higher stability makes it unnecessary to keep the strict cold chain of antibodies, which increases therapeutic costs and limits product half-life (Hemandez et al., 2018, Am J Manag Care, 24(2):109-12). Furthermore, intracellular delivery of these antibody-encoding DNA plasmids achieves stable plasma antibody concentrations for significant time periods, limiting the need of multiple administrations and providing a novel tool for immune therapy of cancer.

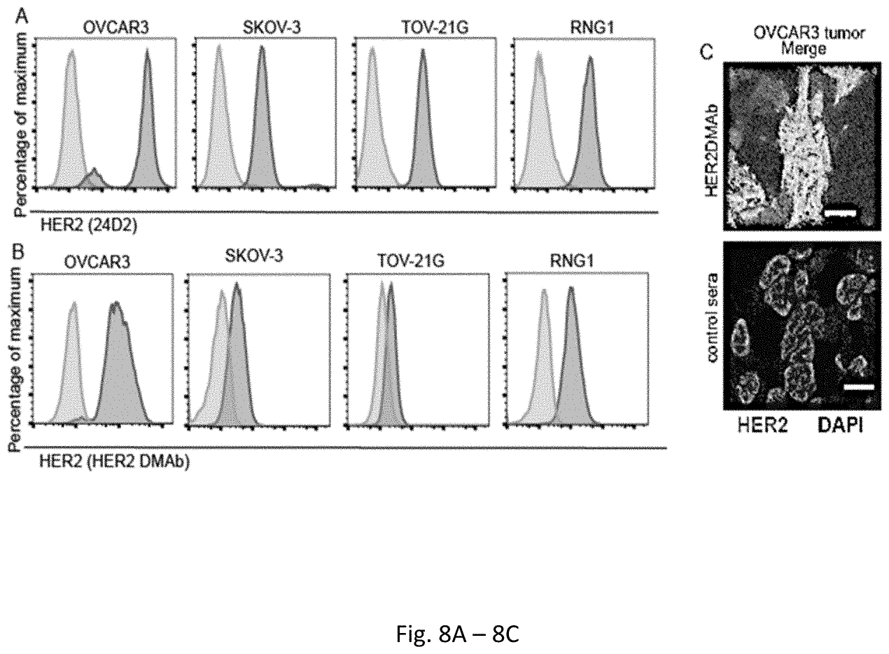

A HER2DMAb was generated by encoding codon and RNA-optimized sequences for the heavy and light chains of pertuzumab into the pVAX1 plasmid expression vector (FIG. 6A). These sequences were preceded by an IgE signal peptide and the heavy and light chains were separated by P2A and furin cleavage sites. Antibody expression was tested in vitro by transfecting 293T cells with DNA encoding the HER2DMAb or an irrelevant protein. 48 hours later the supernatant was collected and a western blot was performed. Bands corresponding to the heavy and light antibody chains were identified in the HER2DMAb transfected 293T supernatant but not in the irrelevant protein control (FIG. 6B). An ELISA was used to determine the amount of human IgG and it was observed that HER2DMAb was expressed by 293T at 5-6 μg/ml which was validated using RD cells (FIG. 7A).

After confirming in vitro expression, expression of the HER2DMAb was confirmed in vivo. 200 μg of HER2DMAb or empty vector was injected, followed by adaptive electroporation, into the tibialis anterior muscle of mice using the CELLECTRA 3P system (Tebas et al., 2017, N Engl J Med, Epub ahead of print). As in the in vitro system, the presence of human IgG was identified in sera from the HER2DMAb injected mice but not in the controls (FIG. 6C) with levels of expression as high as 50 ug/ml in mouse sera and averaging around 25 μg/ml (FIG. 6D).

Next, the ability of the DNA encoded human IgG to bind human HER2 was examined. Plates were coated with human HER2 protein and incubated with sera from the HER2DMAb treated mice or control sera. HER2DMAb from mouse sera bound to human HER2 in a dose-dependent manner (FIG. 6E). To confirm HER2 binding when the protein is present on the cell surface, human HER2 was overexpressed in the murine cell line Brpkp110. HER2DMAb bound human HER2 by flow cytometry only when ectopically expressed (FIG. 6F).

HER2 is Expressed in Human Ovarian Cancer Cell Lines.

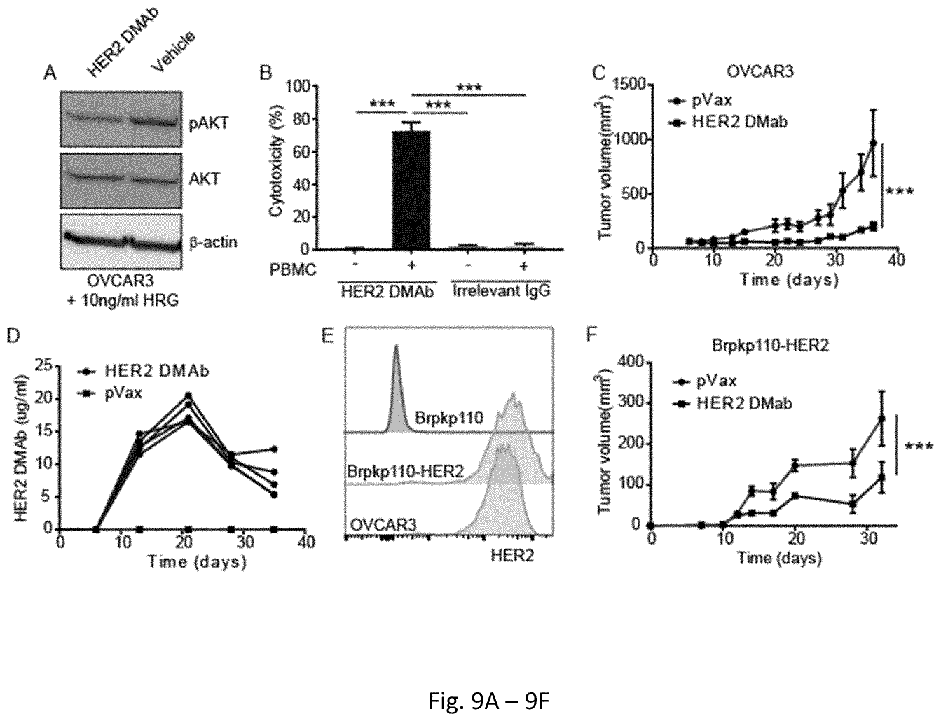

Pertuzumab, unlike trastuzumab, does not require HER2 overexpression in the tumor cell for its anti-tumor activity (Agus et al., 2002, Cancer Cell, 2(2):127-37). In ovarian cancer, pertuzumab has shown a trend towards increase in progression-free survival in cotreatment with gemcitabine and paclitaxel (Kurzeder et al., 2016, J Clin Oncol, 34(21):2516-25). HER2 is overexpressed (histological score 2+/3+) in approximately 11.4% of ovarian cancers (Bookman et al., 2003, J Clin Oncol, 21(2):283-90). To determine if HER2 is also expressed in ovarian cancer cell lines, flow cytometry was performed using a commercial 24D2 antibody (FIG. 8A). The binding of HER2DMAb was validated by doing flow cytometry to these same cells (FIG. 8B). To further validate the in vivo expression and potential targeting of ovarian cancer cell lines using the HER2DMAb, OVCAR3 tumors were generated in mice and immunofluorescence was performed on tumor frozen sections. Positive binding was found using sera from HER2DMAb transfected mice but not with control sera, confirming HER2 in vivo expression and binding of HER2DMAb (FIG. 8C).

HER2DMAb Mediates HER2 Signaling Blockade and Antibody Dependent Cellular Cytotoxicity

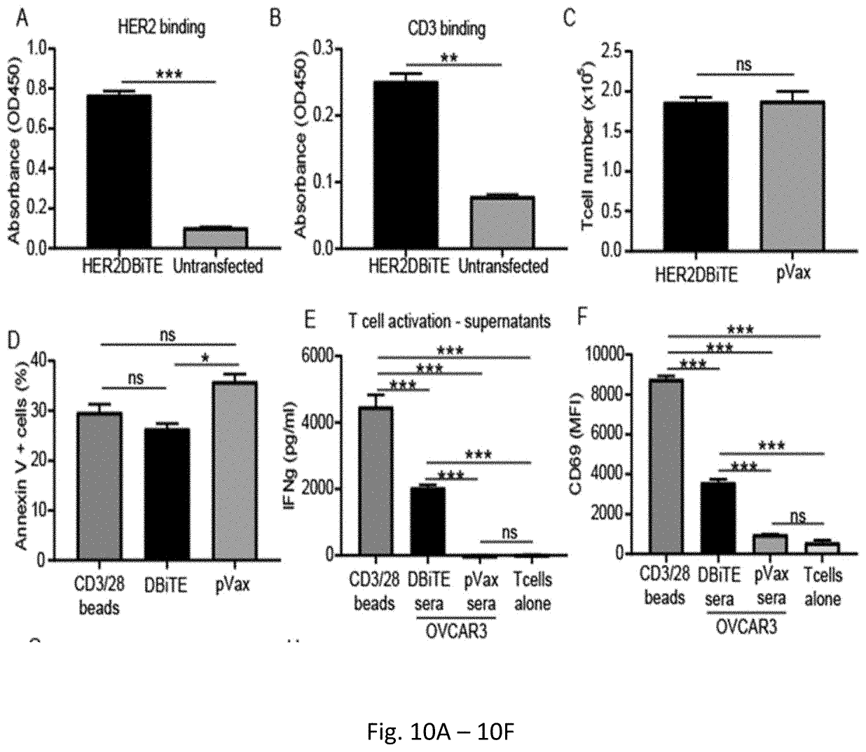

Different mechanisms have been attributed to the anti-tumor effects of anti-cancer antibodies. Pertuzumab acts by preventing HER2 heterodimerization and agonist-mediated signaling (Franklin et al., 2004, Cancer Cell, 5(4):317-28). As expected, HER2DMAb prevented HER2-HER3 agonist heregulin-induced (HRG induced) signaling in OVCAR3 cells, as evidenced by decreased Akt phosphorylation when compared to the vehicle control (FIG. 9A).

Another mechanism by which MAbs have anti-tumor activity is through antibody dependent cellular cytotoxicity (ADCC). To study the ADCC potential of the HER2DMAb, OVCAR3 cells were coincubated together with or without peripheral blood mononuclear cells (PBMCs), in the presence of sera from HER2DMAb or using sera from empty vector treated mice. HER2DMAb sera effectively killed the ovarian cancer cells in the presence of PBMC, but not in their absence. In addition, no killing was observed in the control sera conditions (FIG. 9B and FIG. 7B) or against HER2-cell lines, such as MDA-MB-231 (FIG. 7C). Similarly, HER2dMAb showed antibody-dependent phagocytosis activity (FIG. 7D).

HER2DMAb Delays Cancer Progression In Vivo.

To determine the anti-tumor effects of HER2DMAb in vivo, mice were challenged with the OVCAR-3 ovarian cancer cell line. Nude mice have no T cells but present enhanced NK and macrophage activity, and their splenocytes can lyse OVCAR3 in vitro in the presence of HER2dMAb (FIG. 7E). 100 μg of HER2DMAb or empty vector was delivered to the muscle by EP when tumors reached an average of 50 mm3. HER2DMAb injected animals demonstrated a significant delay in tumor growth resulting in improved survival (FIG. 9C). HER2DMAb antibody levels peaked 2 weeks after DMAb injection with levels of around 20 ug/ml, and sustained levels of around 5-10 ug/ml over a month through the end of the experiment (FIG. 9D). To validate the anti-tumor effect in an immunocompetent host, which would better mimic a clinical administration, tumors were generated using the murine human HER2 breast cancer cell line Brkpk110. This cell line was engineered to express similar HER2 levels to OVCAR3 (FIG. 9E). 5 days after tumor challenge, the mice were treated with HER2DMAb or the empty vector. HER2DMAb also delayed tumor progression in this aggressive model of breast cancer (FIG. 9F).

Upon studying the kinetics of HER2dMAb, it was noted that there was a decrease of antibody expression over almost 300 days. To investigate this phenomenon, the induction of antibodies against this human construct expressed in mice was evaluated. Development of anti-HER2dMAb antibody was observed in sera after treatment with HER2dMAb (FIG. 7F), which may contribute to its decline over time.

Generation, Expression and Cytotoxicity of HER2BiTE

Bispecific T cell engagers (BiTEs) have 2 binding antibody fragments (scFv) so that one of them engages the tumor antigen and the other activates by binding to T cells driving CD3 activation. Despite high antitumor activity, BiTE therapy has advanced slowly as these new tools have major limitations due to their in vivo elimination half-life of approximately 2.1 hours. This short half-life imposes BiTE therapy to be administered continuous intravenous infusion with an infusion pump for 4-8 weeks per cycle. Recent experiments with RNA expressing BiTEs have shown expression for up to 6 days following IV infusion, a considerable advance (Stadler et al., 2017, Nat Med, 23(7):815-7).

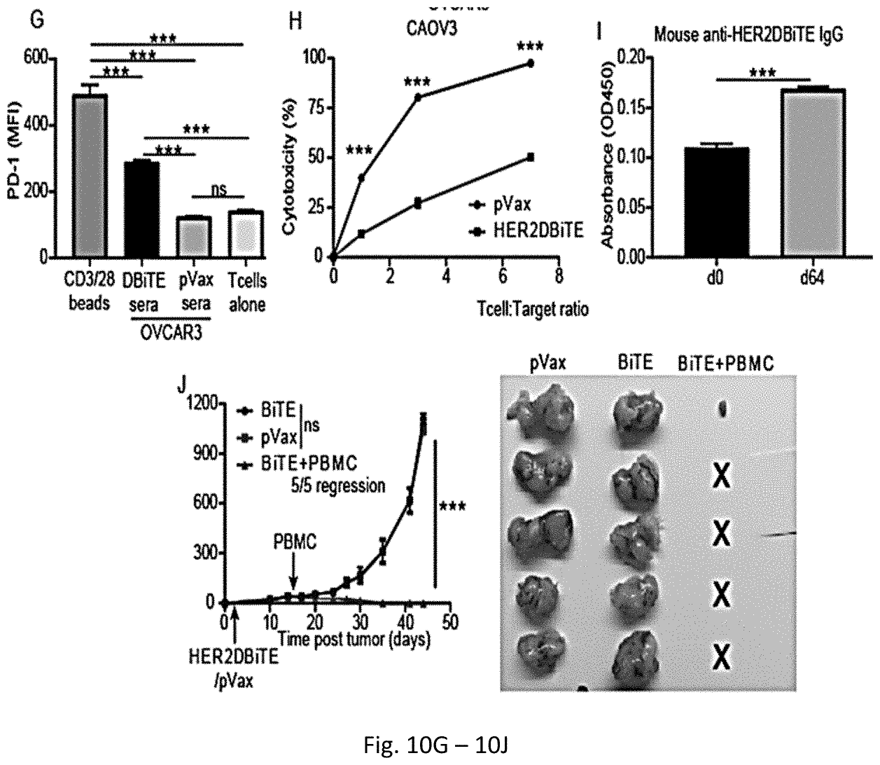

An optimized HER2BiTE was generated by fusing the scFv of the HER2DMAb with the scFv of the stimulatory antibody anti-CD3 (OKT3) (FIG. 11A). The HER2BiTE efficiently expressed in vivo upon injection and electroporation into the mouse tibialis anterior muscle (FIG. 11B). The new HER2DBiTE retained binding to HER2 and bound to CD3 (FIGS. 10A and 10B). Importantly, although the stimulation UCHT1 provides has been reported capable of killing T cells, an increased proportion of apoptosis or difference in T cell numbers was not observed when OKT3 cells were cocultured with HER2DBiTE as compared with just control in the presence of HER2+ cells (FIG. 10C and FIG. 10D). To determine the functionality of the HER2DBiTE expressed in vivo, we cultured sera from mice injected with HER2DBiTE or empty vector with HER2+ ovarian cancer cells and T cells. Sera from mice after HER2DBiTE treatment showed T cell activation (FIG. 10E through FIG. 10G) and an efficient dose-dependent cytotoxicity of OVCAR3 and CAOV3 cells. No cytotoxicity was found upon incubation with sera from empty vector-treated mice or was observed in the absence of T cells (FIG. 11C and FIG. 10H). Incubation of T cells with OVCAR3 at a 1:5 ratio with 5% sera (5 μl in 100 μl) from treated mice showed that the DBiTEs exhibited potent activity for approximately 4 months (FIG. 11D). As in HER2DBiTE, generation of anti-HER2DBiTE antibodies was observed, which could in part be responsible for the circulating levels over time (FIG. 10I). To determine the DBiTE antitumor activity in vivo, NOD/SCID-γ (NSG) mice were challenged with OVCAR3. Mice were treated with a single administration of 200 μg of HER2DBiTE or empty vector a day after tumor implantation. Two weeks after tumor inoculation, when tumors were approximately 50 mm³, 10,000,000 PBMCs were injected intraperitoneally into each mouse. HER2DBiTE treatment significantly affected tumor progression (FIG. 11E), with tumor regression or tumor elimination observed in 8 out of 10 tumors while no tumor impact was observed in the control group (FIG. 11F).

No in vivo effect of HER2DBiTE was observed in the absence of PBMCs (FIG. 10J). HER2DBiTE expressed in vivo for approximately 4 months delivered by a simple injection lasting just seconds and presented a dramatic antitumor activity. Synthetic DNA delivery of BiTEs could alleviate the burden generated by the short half-life of BiTE therapy and provide new applications for this tool in cancer immune therapy.

Together, the data demonstrate that DMAbs can encode HER2DMAb and HER2DBiTEs allowing for them to be durably expressed in vivo at high levels and drive potent anti-tumor activity. This approach provides valuable new tools for the treatment of ovarian as well as potentially other cancers.

### Example 3

EGFRvIII-Targeting DNA-Encoded Immune Cell Engager (DICE) Generates In Vivo Expression of Bispecific Antibody that Induces T Cell-Mediated Cytolytic Activities Against EGFRvIII-Positive Tumors and Controls Tumor Growth in a GBM Mouse Model

Development of bispecific antibodies targeting T cells and tumor-associated antigens (TAAs) has exponentially expanded in both preclinical and clinical settings in recent years. In 2017, one bispecific antibody was approved to treat acute lymphoblastic leukemia. However, due to its low molecular weight, the serum half-life of the antibody is only about 4 hours. As a result, the treatment requires a continuous IV injection of the antibody over several days, which can extend to several weeks. Poor pharmacokinetic profile has presented a big challenge in development of bispecific antibodies, along with other difficulties associated with manufacturing and molecule stability. To address these problems, optimized synthetic DNA-encoded immune cell engagers (DICE) were developed which are designed to express bispecific antibodies in vivo. Mice given a single administration of HER2-DICE exhibit long-term in vivo expression of the bispecific antibody and T cell-mediated cytolytic activities against a HER2-expressing ovarian cell line for over 120 days. In the same study, HER2-DICE not only controlled tumor progression but promoted tumor clearance in many of animals in an ovarian cancer mouse model. With a similar strategy, a DICE targeting EGFRvIII, a TAA which is expressed in 30-50% of glioblastoma multiform (GBM) patients was developed. Supernatant samples from cells transfected with EGFRvIII-DICE in vitro showed potent target-specific binding affinity to both EGFRvIII and CD3, and induced T cell-mediated cytolytic activity against a GBM cell line overexpressing EGFRvIII. Co-culturing target cells and primary human T cells in the presence of EGFRvIII-DICE supernatant stimulated robust T cell responses and displayed significant levels of IFNγ, TNFα, and CD107a in cytotoxic T cell population. Finally, in a GBM mouse challenge model, treatment of EGFRvIII-DICE to NSG mice repopulated with human T cells resulted in control of tumor growth, which was not observed in the empty vector control group. These studies support that synthetic DNA delivery of bispecific antibody generates potent and functional antibodies that are capable of invigorating cytotoxic T cell function and could be studied as an alternative approach to development of bispecific antibodies for cancer immunotherapy.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

1. A nucleic acid molecule encoding one or more synthetic DNA encoded bispecific immune cell engager, wherein the more synthetic DNA encoded bispecific immune cell engager comprises at least one least one antigen binding domain, and at least one immune cell engaging domain, wherein the antigen binding domain targets at least one antigen selected from the group consisting of CD19, B-cell maturation antigen (BCMA), CD33, Fibroblast Activation Protein (FAP), follicle stimulating hormone receptor (FSHR), epidermal growth factor receptor (EGFR), prostate-specific membrane antigen (PSMA), CD123 and human epidermal growth factor receptor 2 (Her2).

2. (canceled)

3. The nucleic acid molecule of claim 1, wherein the immune cell engaging domain targets a cell selected from the group consisting of a T cell, an antigen presenting cell, a natural killer (NK) cell, a neutrophil and a macrophage.

4. The nucleic acid molecule of claim 1, wherein the immune cell engaging domain targets at least one T cell specific receptor molecule selected from the group consisting of CD3, the T cell receptor (TCR), CD28, CD16, NKG2D, Ox40, 4-1BB, CD2, CD5, CD40, FcgRs, FceRs, FcaRs and CD95.

5. The nucleic acid molecule of claim 4, wherein the immune cell engaging domain targets CD3.

6. The nucleic acid molecule of claim 1 comprising a nucleotide sequence encoding one or more sequences selected from the group consisting of:
a) an amino acid sequence having at least about 90% identity over an entire length of the amino acid sequence to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:70, SEQ ID NO:72, SEQ ID NO:74 or SEQ ID NO:76;
b) a fragment of an amino acid sequence having at least about 90% identity over at least 65% of the amino acid sequence to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:70, SEQ ID NO:72, SEQ ID NO:74 or SEQ ID NO:76;
c) an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO: 14, SEQ ID NO:16, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:70, SEQ ID NO:72, SEQ ID NO:74 or SEQ ID NO:76; and
d) a fragment of an amino acid sequence comprising at least 65% of an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:70, SEQ ID NO:72, SEQ ID NO:74 or SEQ ID NO:76.

7. The nucleic acid molecule of claim 1, selected from the group consisting of:
a) a nucleotide sequence having at least about 90% identity over an entire length of the nucleic acid sequence to a nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:69, SEQ ID NO:71, SEQ ID NO:73, and SEQ ID NO:75;
b) a fragment of a nucleotide sequence having at least about 90% identity over at least 65% of the nucleic acid sequence to a nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:69, SEQ ID NO:71, SEQ ID NO:73, and SEQ ID NO:75;
c) a nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:69, SEQ ID NO:71, SEQ ID NO:73, and SEQ ID NO:75; and
d) a fragment of a nucleotide sequence comprising at least 65% of a nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:69, SEQ ID NO:71, SEQ ID NO:73, and SEQ ID NO:75.

8. The nucleic acid molecule of claim 1, wherein the nucleotide sequence is operably linked to a nucleic acid sequence encoding an IgE leader sequence.

9. The nucleic acid molecule of claim 1, wherein the nucleic acid molecule comprises an expression vector.

10. A composition comprising the nucleic acid molecule of claim 1.

11. The composition of claim 10, further comprising a pharmaceutically acceptable excipient.

12. A method of preventing or treating a disease or disorder in a subject, the method comprising administering to the subject a nucleic acid molecule of claim 1 or a composition comprising the same.

13. The method of claim 12, wherein the disease is selected from the group consisting of a benign tumor, cancer and a cancer-associated disease.

14. A nucleic acid molecule encoding one or more synthetic antibodies, wherein the nucleic acid molecule comprises at least one selected from the group consisting of:
a) a nucleotide sequence encoding an anti-human epidermal growth factor receptor 2 (HER2) synthetic antibody;
b) a nucleotide sequence encoding a fragment of an anti-HER2 synthetic antibody;
c) a nucleotide sequence encoding a ScFv anti-HER2 synthetic antibody; and
d) a nucleotide sequence encoding a fragment of a ScFv anti-HER2 synthetic antibody.

15. The nucleic acid molecule of claim 14, comprising a nucleotide sequence encoding one or more sequences selected from the group consisting of:
a) an amino acid sequence having at least about 90% identity over an entire length of the amino acid sequence to an amino acid sequence selected from the group consisting of SEQ ID NO:62, SEQ ID NO:64, and SEQ ID NO:66;
b) a fragment of an amino acid sequence having at least about 90% identity over at least 65% of the amino acid sequence to an amino acid sequence selected from the group consisting of SEQ ID NO:62, SEQ ID NO:64, and SEQ ID NO:66;
c) an amino acid sequence selected from the group consisting of SEQ ID NO:62, SEQ ID NO:64, and SEQ ID NO:66; and
d) a fragment of an amino acid sequence comprising at least 65% of an amino acid sequence selected from the group consisting of SEQ ID NO:62, SEQ ID NO:64, and SEQ ID NO:66.

16. The nucleic acid molecule of claim 14, selected from the group consisting of:
a) a nucleotide sequence having at least about 90% identity over an entire length of the nucleic acid sequence to a nucleotide sequence selected from the group consisting of SEQ ID NO:61, SEQ ID NO:63, and SEQ ID NO:65;
b) a fragment of a nucleotide sequence having at least about 90% identity over at least 65% of the nucleic acid sequence to a nucleotide sequence selected from the group consisting of SEQ ID NO:61, SEQ ID NO:63, and SEQ ID NO:65;
c) a nucleotide sequence selected from the group consisting of SEQ ID NO:61, SEQ ID NO:63, and SEQ ID NO:65; and
d) a fragment of a nucleotide sequence comprising at least 65% of a nucleotide sequence selected from the group consisting of SEQ ID NO:61, SEQ ID NO:63, and SEQ ID NO:65.

17. The nucleic acid molecule of claim 14, wherein the nucleotide sequence is operably linked to a nucleic acid sequence encoding an IgE leader sequence.

18. The nucleic acid molecule of claim 14, wherein the nucleic acid molecule comprises an expression vector.

19. A composition comprising the nucleic acid molecule of claim 14.

20. The composition of claim 19, further comprising a pharmaceutically acceptable excipient.

21. A method of preventing or treating a disease associated with HER2 expression in a subject, the method comprising administering to the subject the nucleic acid molecule of claim 14 or a composition comprising the same.

22.-23. (canceled)
